(19) United States
(12) Patent Application Publication
(10) Pub. No.: US 2026/0275356 A1
(43) Pub. Date: Sep. 17, 2026

(54) ANTISENSE OLIGONUCLEOTIDES FOR THE TREATMENT OF LIVER DISEASE

(71) Applicant: ProQR Therapeutics II B.V., Leiden (NL)

(72) Inventors: Maarten HOLKERS, Leiden (NL); Bart KLEIN, Leiden (NL); Christopher Kuyler DOYLE, Leiden (NL); Bruno Filipe Madeira DE ALBUQUERQUE, Leiden (NL); Marko POTMAN, Leiden (NL)

(73) Assignee: ProQR Therapeutics II B.V., Leiden (NL)

(21) Appl. No.: 19/168,351

(22) PCT Filed: Mar. 26, 2024
(86) PCT No.: PCT/EP2024/058159

§ 371 Date: Sep. 24, 2025

(30) Foreign Application Priority Data
Mar. 27, 2023 (GB) 2304438.1
Nov. 27, 2023 (GB) 2318087.0

(51) Int. Cl. C12N 15/113; C12N 15/86; C12N 15/88

(52) U.S. Cl. CPC C12N 15/1138; C12N 15/86; C12N 15/88; C12N 2310/11; C12N 2310/314; C12N 2310/334; C12N 2310/351; C12N 2750/14143

(57) ABSTRACT

The present invention relates to antisense oligonucleotides (AONs) that can mediate RNA editing by binding to a target RNA nucleic acid molecule, preferably an RNA transcript molecule, in a cell and recruiting an endogenous deaminating enzyme in the cell to deaminate one or more target adenosine nucleotides in the target RNA molecule to an inosine. The target RNA molecule is a transcript molecule form the SLC10A1 gene that encodes the Na/Taurocholate Co-transporting Polypeptide (NTCP). The RNA editing of the one or more target adenosines will result in a loss-of-function of the NTCP protein, which will result in lowered uptake of bile acids from the portal circulation into the liver, thereby lowering the risk of suffering from disorders related to bile accumulation in the liver.

### TECHNICAL FIELD

This disclosure relates to the field of medicine, and in particular to the field of (chronic) liver diseases, like liver cirrhosis, for example caused by non-alcoholic fatty liver disease (NAFLD) and bile accumulation. The disclosure describes antisense oligonucleotides that mediate nucleotide-specific RNA editing in the SLC10A1 gene transcript to bring about amino acid changes of the encoded Na₊/Taurocholate Co-transporting Polypeptide (NTCP) that influence its activity.

### BACKGROUND

Cholestatic disorders are caused by a buildup of bile acids in the liver due to bile duct dysfunction, which causes liver cell damage. The consequences of these disorders can be devastating and significantly impact a person's quality of life. Manifestations include pruritus, dry skin, fatigue, pain, weight loss, and many others. Without treatment, the damage progresses through various stages, from fibrosis to cirrhosis, ultimately leading to liver failure and an increased risk of liver cancer. Liver transplants are often necessary for primary sclerosing cholangitis (PSC) and biliary atresia (BA), two forms of cholestatic disease with high unmet medical needs. PSC is a condition that causes inflammation and is typically diagnosed in people aged 30 to 40, more commonly affecting men (66%). It is estimated that approximately 80,000 people in North America and Europe suffer from PSC, with a prevalence of 1 to 9 individuals per 100,000. This condition causes fibrosis and sclerosis of bile ducts, leading to a toxic buildup of bile acids in the liver. BA is a pediatric condition that affects newborns, resulting from the absence or defect of bile ducts. This condition causes harmful bile acids to accumulate in the liver, leading to rapid progression to cirrhosis early in life. It is estimated that approximately 20,000 individuals in North America and Europe suffer from BA, with a prevalence of 1 in 10,000 to 15,000 newborns in the western world.

Currently, there are no approved drugs for treating PSC or BA. For PSC, liver transplantation is the only treatment option with evidence to extend survival. However, PSC can return in 20 to 40% of patients who undergo liver transplantation, and the median survival without a transplant is only approximately 21 years. Surgery in the first weeks of life for BA is the standard treatment. However, most patients who receive this surgery will still require a liver transplant early in life.

Hepatocytes in the liver predominantly obtain bile acids from the enterohepatic reuptake cycle. The process of taking up bile acids from the portal circulation into hepatocytes is primarily carried out by a transporter protein named Na⁺/Taurocholate Co-transporting Polypeptide (NTCP), encoded by the SLC10A1 gene. The NTCP protein has received significant attention because it has been identified as the main protein involved in recognition and entry of Hepatitis B Virus (HBV) and Hepatitis D Virus (HDV). However, the normal function of NTCP at least relates to taking up conjugated bile acids from the circulation into hepatocytes. As indicated above, an accumulation of bile acids in the liver has been associated with a wide variety of liver diseases and it has been shown that inhibiting NTCP can improve liver function by reducing the intrahepatocellular levels of toxic bile acids, and preventing increased liver damage using certain available markers, such as fibrosis, cholangiocyte proliferation, Alkaline phosphatase (ALP), alanine transaminase (ALT), and inflammation biomarkers like cytokines (Slijepcevic D and Van de Graaf 2017. Dig Dis. 35(3):251-258; Slijepcevic D et al. 2018. Hepatology 68(3):1057-1069).

The present disclosure relates to a completely different approach of lowering the activity of NTCP, namely by using antisense oligonucleotide (AONs) and the cell's own nucleic acid editing machinery in order to specifically target and amend one or more nucleotides in the SLC10A1 transcript, thereby providing an NTCP protein with a loss-of-function, which in turn should lower the ability of hepatocytes to take up bile acids from the portal circulation and through this, treat disorders related to bile accumulation in the liver. The technology that the present disclosure relates to is generally referred to as ‘RNA editing’.

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A) to inosine (I) conversions and cytidine (C) to uridine (U) conversions, which occur through enzymes called Adenosine Deaminases acting on RNA (ADAR) and APOBEC/AID (cytidine deaminases that act on RNA), respectively.

ADAR is a multi-domain protein, comprising of a catalytic domain and two to three double-stranded RNA recognition domains, depending on the enzyme in question. Each recognition domain recognizes a specific double-stranded RNA (dsRNA) sequence and/or conformation. The catalytic domain does also play a role in recognizing and binding a part of the dsRNA helix, although the key function of the catalytic domain is to convert an A into an I in a nearby, predefined, position in the target RNA, by deamination of the nucleobase. Inosine is read as guanosine by the translational machinery of the cell, meaning that, if an edited adenosine is in a coding region of an mRNA or pre-mRNA, it can recode the protein sequence. A-to-I conversions may also occur in 5′ non-coding sequences of a target mRNA, creating new translational start sites upstream of the original start site, which gives rise to N-terminally extended proteins, or in the 3′ untranslated region (UTR) or other non-coding parts of the transcript, which may affect the processing and/or stability of the RNA. In addition, A-to-I conversions may take place in splice elements in introns or exons in pre-mRNAs, thereby altering the pattern of splicing. As a result, exons may be included or skipped. The enzymes catalysing adenosine deamination are within an enzyme family of ADARs, which include human deaminases hADAR1 and hADAR2, as well as hADAR3. However, for hADAR3 no deaminase activity has been demonstrated.

The use of oligonucleotides to edit a target RNA, applying adenosine deaminase, has been described (e.g., Woolf et al. Proc Natl Acad Sci USA 1995, 92:8298-8302; Montiel-Gonzalez et al. Proc Natl Acad Sci USA 2013, 110 (45): 18285-18290; Vogel et al. Angewandte Chemie Int 2014, Ed 53:267-271). A disadvantage of the method described by Montiel-Gonzalez et al. (2013) is the need for a fusion protein consisting of the boxB recognition domain of bacteriophage Lambda N-protein, fused to the adenosine deaminase domain of a truncated natural ADAR protein. It requires target cells to be either transduced with the fusion protein, which is a major hurdle, or that target cells are transfected with a nucleic acid construct encoding the engineered adenosine deaminase fusion protein for expression. The system described by Vogel et al. (2014) suffers from similar drawbacks, in that it is not clear how to apply the system without having to genetically modify the ADAR first and subsequently transfect or transform the cells harboring the target RNA, to provide the cells with this genetically engineered protein. U.S. Pat. No. 9,650,627 describes a similar system. The oligonucleotides of Woolf et al. (1995) that were 100% complementary to the target RNA sequences, suffered from severe lack of specificity: nearly all adenosines in the target RNA strand that were complementary to the AON were edited.

It is known that ADAR may act on any dsRNA. Through a process sometimes referred to as ‘promiscuous editing’, the enzyme will edit multiple A's in the dsRNA. Hence, there was a need for methods and means that circumvent such promiscuous editing and only target specific adenosines in a target RNA molecule to become therapeutic applicable. Vogel et al. (2014) showed that such off-target editing can be suppressed by using 2′-O-methyl(2′-OMe) modified nucleosides in the oligonucleotide at positions opposite to adenosines that should not be edited and used a non-modified nucleoside directly opposite to the specifically targeted adenosine on the target RNA. However, the specific editing effect at the target nucleotide has not been shown to take place without the use of recombinant ADAR enzymes having covalent bonds with the AON. Several publications have now shown that the recruitment of endogenous ADAR (hence without the need for an exogenous and/or recombinant source) is feasible while maintaining a specificity in which a single adenosine within a target RNA molecule can be targeted and deaminated to an inosine. WO2016/097212 discloses AONs for the targeted editing of RNA, wherein the AONs are characterized by a sequence that is complementary to a target RNA sequence (therein referred to as the ‘targeting portion’) and by the presence of a stem-loop/hairpin structure (therein referred to as the ‘recruitment portion’), which is preferably non-complementary to the target RNA. Such oligonucleotides are referred to as ‘self-looping AONs’. The recruitment portion acts in recruiting a natural ADAR enzyme present in the cell (endogenously present) to the dsRNA formed by hybridization of the target sequence with the targeting portion. Due to the recruitment portion, there is no need for conjugated entities or presence of modified recombinant ADAR enzymes. WO2016/097212 describes the recruitment portion as being a stem-loop structure mimicking either a natural substrate (e.g., the GluB receptor) or a Z-DNA structure known to be recognized by the dsRNA binding domains, or Z-DNA binding domains, of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described is an intramolecular stem-loop structure, formed within the AON itself, and are thought to attract (endogenous) ADAR. Similar stem-loop structure-comprising systems for RNA editing have since then been described in WO2017/050306, WO2020/001793, WO2017/010556, U.S. Pat. No. 11,390,865, WO2020/246560, and WO2022/078995.

WO2017/220751 and WO2018/041973 describe a next generation type of AONs that do not comprise such a stem-loop structure but that are (almost fully) complementary to the targeted area, and that appeared still capable of attracting endogenous ADAR enzymes. In one embodiment, one or more mismatching nucleotides, wobbles, or bulges exist between the oligonucleotide and the target sequence. A sole mismatch may be at the site of the nucleoside opposite the target adenosine, but in other embodiments AONs (or “RNA editing oligonucleotides”—even though the deamination reaction is carried out by the ADAR enzyme—and often abbreviated to ‘EONs’) were described with multiple bulges and/or wobbles when attached to the target sequence area. It appeared possible to achieve in vitro, ex vivo and in vivo RNA editing with AONs lacking a stem-loop structure and with endogenous ADAR enzymes when the sequence of the AON was carefully selected such that it could attract/recruit ADAR. The ‘orphan nucleoside’, which is defined as the nucleoside in the AON that is positioned directly opposite the target adenosine in the target RNA molecule, was a nucleotide with an unmodified cytosine nucleobase and that did not carry a 2′-OMe modification. The orphan nucleoside can be a deoxyribonucleoside (DNA), wherein the remainder of the AON could still carry 2′-O-alkyl modifications at the sugar entity (such as 2′-OMe), or the nucleotides directly surrounding the orphan nucleoside contained chemical modifications (such as DNA in comparison to RNA) that further improved the RNA editing efficiency and/or increased the resistance against nucleases. Such effects could even be further improved by using sense oligonucleotides (SONs) that ‘protected’ the AONs against breakdown upon delivery to the cells (described in WO2018/134301 and U.S. Pat. No. 11,274,300).

The use of chemical modifications and particular structures in oligonucleotides that could be used in ADAR-mediated editing of specific adenosines in a target RNA have been the subject of numerous disclosures in the field, such as WO2019/111957, WO2019/158475, WO2020/165077, WO2020/201406, WO2020/211780, WO2021/008447, WO2021/020550, WO2021/060527, WO2021/117729, WO2021/136408, WO2021/182474, WO2021/216853, WO2021/242778, WO2021/242870, WO2021/242889, WO2022/007803, WO2022/018207, WO2022/026928, and WO2022/124345. The use of specific sugar moieties has been disclosed in for instance WO2020/154342, WO2020/154343, WO2020/154344, WO2022/103839, and WO2022/103852, whereas the use of stereo-defined linker moieties (in general for oligonucleotides that for instance can be used for exon skipping, in gapmers, in siRNA, or specifically for RNA-editing oligonucleotides, related to a wide variety of target sequences) has been described in WO2011/005761, WO2014/010250, WO2014/012081, WO2015/107425, WO2017/015575 (HTT), WO2017/062862, WO2017/160741, WO2017/192664, WO2017/192679 (DMD), WO2017/198775, WO2017/210647, WO2018/067973, WO2018/098264, WO2018/223056 (PNPLA3), WO2018/223073 (APOC3), WO2018/223081 (PNPLA3), WO2018/237194, WO2019/032607 (C9orf72), WO2019/055951, WO2019/075357 (SMA/ALS), WO2019/200185 (DM1), WO2019/217784 (DM1), WO2019/219581, WO2020/118246 (DM1), WO2020/160336 (HTT), WO2020/191252, WO2020/196662, WO2020/219981 (USH2A), WO2020/219983 (RHO), WO2020/227691 (C9orf72), WO2021/071788 (C9orf72), WO2021/071858, WO2021/178237 (MAPT), WO2021/234459, WO2021/237223, WO2022/099159, WO2021/030778, WO2022/174053, and WO2023/278589. Next to these disclosures, an extensive number of publications relate to the targeting of specific RNA target molecules, or specific adenosines within such RNA target molecules, be it to repair a mutation that resulted in a premature stop codon, or other mutation causing disease. Examples of such disclosures in which adenosines are targeted within specified target RNA molecules are WO2020/157008 and WO2021/136404 (USH2A); WO2021/113270 (APP); WO2021/113390 (CMT1A); WO2021/209010 (IDUA, Hurler syndrome); WO2021/231673 and WO2021/242903 (LRRK2); WO2021/231675 (ASS1); WO2021/231679 (GJB2); WO2019/071274 and WO2021/231680 (MECP2); WO2021/231685 and WO2021/231692 (OTOF, autosomal recessive non-syndromic hearing loss); WO2021/231691 (XLRS); WO2021/231698 (argininosuccinate lyase deficiency); WO2021/130313 and WO2021/231830 (ABCA4); and WO2021/243023 (SERPINA1).

The present invention aims to provide one or more alternative, and/or improved, compounds or compositions for use in the treatment of liver disease, such as cholestatic disorders caused by bile acid accumulation in the liver.

### SUMMARY OF THE INVENTION

Disclosed herein is an antisense oligonucleotide (AON) that is capable of recruiting an endogenous ADAR enzyme in a human cell after the AON has formed a double-stranded complex with a region of a target RNA nucleic acid molecule in a cell, wherein the region comprises a target adenosine, wherein the nucleotide in the AON that is opposite the target adenosine is the orphan nucleotide, wherein the ADAR enzyme can deaminate the target adenosine into an inosine after binding to the double-stranded complex, and wherein the target RNA nucleic acid molecule is a transcript molecule of the human SLC10A1 gene encoding the Na⁺/Taurocholate Co-transporting Polypeptide (NTCP). Preferably, the transcript molecule is a pre-mRNA or an mRNA molecule. Preferably, the cell is a liver cell, more preferably a hepatocyte. In one aspect, the SLC10A1 gene is wildtype, and the target adenosine is selected from the group consisting of: (i) the adenosine in the CAG codon coding for glutamine (Q) at position 68 of the NTCP protein, and wherein the deamination of the adenosine changes the amino acid to an arginine (R); (ii) the adenosine is the first adenosine in the CAA codon coding for glutamine (Q) at position 261 of the NTCP protein, and wherein the deamination of the adenosine changes the amino acid to an arginine (R); (iii) the adenosine is in the GAG codon coding for glutamic acid (E) at position 257 of the NTCP protein, and wherein the deamination of the adenosine changes the amino acid to glycine (G); and (iv) the adenosine is the first adenosine in the AAG codon coding for lysine (K) at position 314 of the NTCP protein, and wherein the deamination of the adenosine changes the amino acid to glutamic acid (E); wherein the deamination of the target adenosine results in an NTCP protein that is impaired in its function to transport bile acids from portal circulation into the cell. In one aspect, the orphan nucleotide is a deoxycytidine or a deoxyuridine. In one aspect, the orphan nucleotide is a cytidine analog such as a deoxynucleotide comprising a 6-amino-5-nitro-3-yl-2 (1H)-pyridone nucleobase. In one aspect, the orphan nucleotide is a uridine analog such as a deoxynucleotide comprising an iso-uracil nucleobase. In one aspect the nucleotide numbering in the AON is such that the orphan nucleotide is number 0 and nucleotides are further positively (+) incremented towards the 5′ end and negatively (−) incremented towards the 3′ end, and the first nucleotide 3′ from the orphan nucleotide (−1) is a deoxyinosine when the nucleotide opposite this position is a cytidine in the target RNA nucleic acid molecule.

Disclosed is an AON, wherein the AON comprises one or more modifications in the linkage moiety, which is each independently selected from a phosphorothioate (PS), phosphonoacetate, phosphorodithioate, methyl phosphonate (MP), sulfonylphosphoramidate, a (1,3-dimethylimidazolidin-2-ylidene) phosphoramidate (PNdmi) linkage, or a PNms linkage with the structure according to formula (I), wherein: X═O or S; and R=an aryl, a substituted aryl, a heterocycle, a substituted heterocycle, an aromatic heterocycle, a substituted aromatic heterocycle, a C₁-C₆ alkoxy, a substituted C₁-C₆ alkoxy, a C₁-C₂₀ alkyl, a substituted C₁-C₂₀ alkyl, a C₁-C₆ alkenyl, a C₁-C₆ substituted alkenyl, a C₁-C₆ alkynyl, a substituted C₁-C₆ alkynyl, or a conjugate group.

Disclosed is an AON, wherein the internucleoside linkage numbering in the AON is such that linkage number 0 is the linkage 5′ from the orphan nucleotide, and the linkage positions in the oligonucleotide are positively (+) incremented towards the 5′ end and negatively (−) incremented towards the 3′ end, and wherein linkage position-2 is an MP linkage.

Disclosed is an AON, wherein the AON comprises one or more nucleotides comprising a mono- or di-substitution at the 2′, 3′ and/or 5′ position of the ribose, each independently selected from the group consisting of: —OH; —F; substituted or unsubstituted, linear or branched lower (C₁-C₁₀)alkyl, alkenyl, alkynyl, alkaryl, allyl, or aralkyl, that may be interrupted by one or more heteroatoms;—O—, S—, or N-alkyl;—O—, S—, or N-alkenyl;—O—, S—, or N-alkynyl;—O—, S-, or N-allyl;—O-alkyl-O-alkyl; -methoxy; -aminopropoxy; -methoxyethoxy; -dimethylamino oxyethoxy; and -dimethylaminoethoxyethoxy. In one aspect, the AON is (non)covalently bound to a GalNAc moiety.

Disclosed herein is a pharmaceutical composition comprising an AON as disclosed herein, or a vector as disclosed herein, and a pharmaceutically acceptable carrier. Disclosed herein is an AON for use in the treatment of a disease caused by bile accumulation in the liver, such as cholestasis, primary sclerosing cholangitis (PSC), biliary atresia (BA), and liver cirrhosis. Disclosed herein is also an AON for use in the manufacture of a medicament for the treatment of a disease caused by bile accumulation in the liver, such as cholestasis, PSC, BA, and liver cirrhosis.

Disclosed herein is a method of editing a human SLC10A1 polynucleotide in a cell, preferably a hepatocyte, wherein the human SLC10A1 polynucleotide is a pre-mRNA or mRNA molecule, the method comprising contacting the SLC10A1 polynucleotide with an AON capable of triggering an ADAR-mediated adenosine to inosine deamination, thereby editing the SLC10A1 polynucleotide to encode an NTCP protein with a diminished, lowered, or loss of function in bile acid uptake.

Disclosed herein is a method of treating, ameliorating, or slowing down the progression of a disease caused by bile accumulation in the liver, such as cholestasis, PSC, BA, and liver cirrhosis, in a human subject in need thereof, the method comprising administering to said subject an AON as disclosed herein, a vector as disclosed herein, or a pharmaceutical composition as disclosed herein, thereby contacting a SLC10A1 polynucleotide in a cell of the subject with an AON capable of effecting an ADAR-mediated adenosine to inosine deamination, thereby editing the SLC10A1 polynucleotide to encode an NTCP protein with a diminished, lowered, or loss of function in bile acid uptake, thereby treating the subject.

### BRIEF DESCRIPTION OF THE DRAWINGS

One or more embodiments of the invention will now be described, by way of example only, with reference to the accompanying drawings, in which:

FIG. 1 shows part of the 5′ to 3′ sequence of the human SLC10A1 mRNA transcript in which the CAG codon coding for glutamine (Q) at position 68 in the NTCP protein is in bold (SEQ ID NO:1). The underlined adenosine is the target for RNA editing as disclosed herein, resulting in a codon coding for an arginine (R) residue (CIG/CGG) at this position after editing. Below the target sequence the 5′ to 3′ sequences are provided of the 30 initial AONs that were designed to target the target adenosine in SEQ ID NO:1. AONS T1-01 to T1-30 are SEQ ID NO: 5 to SEQ ID NO:34, as indicated. The chemical modifications are as follows: Ae and Ge are 2′-MOE modified adenosine and guanosine, respectively; Cm, Am, Um, and Gm are 2′-OMe modified cytidine, adenosine, uridine, and guanosine, respectively; Gf, Cf, Af, and Uf are 2′-F modified guanosine, cytidine, adenosine, and uridine, respectively; m5Ce is 2′-MOE modified 5-methyl-cytidine; Zd (orphan nucleotide) is a deoxynucleotide (deoxycytidine analog) carrying a Benner's base; Id is deoxyinosine; Te (or m5Ue) is 2′-MOE modified thymidine (identical to a 5-methyl-uridine with a 2′-MOE substitution); “!” refers to a PNdmi linkage; “∧” refers to a MP linkage; “*” refers to a PS linkage. All other internucleoside linkages are phosphodiester (PO) linkages.

FIG. 2 shows part of the sequence of the human SLC10A1 mRNA transcript in which the CAA codon coding for glutamine (Q) at position 261 in the NTCP protein is in bold (SEQ ID NO: 2). The underlined adenosine is the target for RNA editing as disclosed herein, resulting in a codon coding for an arginine (R) residue (CIA/CGA) at this position after editing. Below the target sequence the 5′ to 3′ sequences are provided of the 30 initial AONs that were designed to target the target adenosine in SEQ ID NO:2. AONs T2-01 to T2-30 are SEQ ID NO: 35 to SEQ ID NO:64, as indicated. The chemical modifications are as shown in FIG. 1.

FIG. 3 shows part of the sequence of the human SLC10A1 mRNA transcript in which the GAG codon coding for glutamic acid (E) at position 257 in the NTCP protein is in bold (SEQ ID NO:3). The underlined adenosine is the target for RNA editing as disclosed herein, resulting in a codon coding for a glycine (G) residue (GIG/GGG) at this position after editing. Below the target sequence the 5′ to 3′ sequences are provided of the 30 initial AONs that were designed to target the target adenosine in SEQ ID NO:3. AONs T3-01 to T3-30 are SEQ ID NO: 65 to SEQ ID NO:94, as indicated. The chemical modifications are as shown in FIG. 1.

FIG. 4 shows part of the sequence of the human SLC10A1 mRNA transcript in which the AAG codon coding for lysine (K) at position 314 in the NTCP protein is in bold (SEQ ID NO: 4). The underlined adenosine is the target for RNA editing as disclosed herein, resulting in a codon coding for a glutamic acid (E) residue (IAG/GAG) at this position after editing. Below the target sequence the 5′ to 3′ sequences are provided of the 30 initial AONs that were designed to target the target adenosine in SEQ ID NO:4. AONs T4-01 to T4-30 are SEQ ID NO: 95 to SEQ ID NO: 124, as indicated. The chemical modifications are as shown in FIG. 1.

FIG. 5 shows an additional set of AONs that were designed to target the target adenosine in SEQ ID NO:1 (Q68R). The respective SEQ ID NO's are as indicated. The chemical modifications are as given in FIG. 1, with the symbol “#” representing a PNms linkage and the symbol “θ” representing a PO linkage.

FIG. 6 shows an additional set of AONs that were designed to target the target adenosine in SEQ ID NO:3 (E257G). The respective SEQ ID NO's are as indicated. The chemical modifications are as given in FIG. 1.

FIG. 7A and FIG. 7B show the editing percentages that were measured in two independent experiments, respectively, for the c.203A>G editing (Q68R), using the 16 AONs as indicated in primary human hepatocytes (PHHs) after co-treatment with AG1856 saponin. Two unrelated AONs (RM4777 and RM4266), AG1856 only and non-treated samples served as negative controls.

FIG. 8A and FIG. 8B show the editing percentages that were measured in the two independent experiments, respectively, for the c.770A>G editing (E257G), using the 17 AONs as indicated in PHHs after co-treatment with AG1856 saponin. Two unrelated AONs (RM4777 and RM4266), AG1856 only and non-treated samples served as negative controls.

FIG. 9A and FIG. 9B show the editing percentages that were measured in two independent gymnotic uptake experiments, respectively, for the c.203A>G editing (Q68R), using the 18 AONs as indicated in PHHs in the absence of saponin. Two unrelated AONs (RM4777 and RM4266), and a non-treated sample served as negative controls.

FIG. 10A and FIG. 10B show the editing percentages that were measured in the two independent gymnotic uptake experiments, respectively, separately for the c.770A>G editing (E257G), using the 16 AONs as indicated in PHHs in the absence of saponin. Two unrelated AONs (RM4777 and RM4266), and a non-treated sample served as negative controls.

FIG. 11 shows the editing percentages that were measured in two experiments for the c.203A>G editing (Q68R), using the 16 AONs as indicated in liver spheroids after co-treatment (FIG. 11A) with AG1856 saponin and (FIG. 11B) without co-treatment of saponin. A non-treated sample and AG1856 alone served as negative controls.

FIG. 12 shows the editing percentages that were measured in the two experiments for the c.770A>G editing (E257G), using the 17 AONs as indicated in liver spheroids after co-treatment with AG1856 saponin (FIG. 12A) and without co-treatment of saponin (FIG. 12B). A non-treated sample and AG1856 alone served as negative controls.

FIG. 13 shows the editing percentages that were measured in an experiment for the c.203A>G editing (Q68R), using the 11 AONs as indicated in HepG2 cells over-expressing human NTCP (HepG2NTCP) after co-treatment with AG1856 saponin. A non-treated sample served as a negative control.

FIG. 14 shows the editing percentages that were measured in an experiment for the c.770A>G editing (E257G), using the 10 AONs as indicated in HepG2NTCP cells after co-treatment with AG1856 saponin. A non-treated sample and AG1856 alone served as negative controls.

FIG. 15 shows the editing percentages that were measured in an additional experiment for the c.203A>G editing (Q68R), using the 25 asymmetric AONs as indicated (FIG. 15A), and using the 34 symmetric AONs as indicated (FIG. 15B) in PHHs after co-treatment with AG1856 saponin. An unrelated AON (RM4777) and a non-treated sample served as negative controls.

FIG. 16 shows the editing percentages that were measured in an additional experiment for the c.770A>G editing (E257G), using the 21 asymmetric AONs as indicated (FIG. 16A), and using the 38 symmetric AONs as indicated (FIG. 16B) in PHHs after co-treatment with AG1856 saponin. An unrelated AON (RM4777) and a non-treated sample served as negative controls.

FIG. 17 shows the pmol radiolabelled taurocholic acid (TCA; bile acid) uptake in human U2OS cells after (independent) transfection of plasmids expressing seven human NTCP mutations, as indicated, after treatment with 1.0 μM TCA (FIG. 17A) and 10 μM TCA (FIG. 17B). A wildtype NTCP expressing plasmid was taken as a positive control and a non-treated sample was taken as a negative control.

FIG. 18 shows an additional set of AONs that were designed to target the target adenosine in SEQ ID NO: 1 (Q68R), using a variety of linkages that replace the linkage in the earlier set of AONs. The respective SEQ ID NO's are as indicated, in the second column. The chemical modifications are as given in FIG. 1, with the symbol “#” representing a PNms linkage and the symbol “θ” representing a PO linkage.

FIG. 19 shows the editing percentages in a c.203A>G editing (Q68R) experiment, using the 12 AONs as indicated (details in FIG. 18) in PHHs after transfection using lipofectamine. An unrelated AON (RM4777), a lipofectamine only, and a non-treated (NT) PBS sample served as negative controls.

FIG. 20 shows the editing percentages in a c.203A>G editing (Q68R) experiment, using the 24 AONs as indicated (details in FIG. 18) in PHHs after transfection using lipofectamine. An unrelated AON (CNTRL), a lipofectamine only (Lipo2000), and a non-treated (NT) PBS sample served as negative controls.

FIG. 21 shows the editing percentages in a c.203A>G editing (Q68R) experiment, using the 9 AONs as indicated (details in FIG. 18) in PHHs after co-treatment with AG1856. An unrelated AON (control-ON), a saponin only (AG1856), and a non-treated (NT) sample served as negative controls.

FIG. 22 shows the editing percentages in a c.203A>G editing (Q68R) experiment, using the 26 AONs as indicated (details in FIG. 18) in PHHs after transfection using lipofectamine. An unrelated control (RM4777), a lipofectamine only (MOCK Lipo), and a non-treated (NT) sample served as negative controls.

### DETAILED DESCRIPTION

The AONs as disclosed herein can recruit deaminating enzymes, such as ADAR1 and/or ADAR2 that are endogenously present in a cell. An AON as disclosed herein can mediate RNA editing of a target adenosine present in a target RNA molecule after it is bound to the target RNA molecule, since the deaminating enzymes are recruited to the double-stranded AON/target RNA molecule complex and subsequently deaminate the target adenosine into an inosine.

The oligonucleotides are herein abbreviated to “AONs”, but sometimes also referred to as ‘editing oligonucleotides’, or ‘EONs’, even though the RNA editing event is performed by the deamination enzyme and the action of the oligonucleotide only triggers the RNA editing to take place. There is a constant need for improving the pharmacokinetic properties of the AONs without negatively affecting the efficiency in which the target adenosine is edited in the target RNA, and/or without negatively affecting the stability of the AON itself, which is constantly prone to breakdown because of nucleases present in a natural cell. Many chemical modifications are available for the generation of AONs (and many have been applied in the art). However, many of these properties are not always compatible with the desire of achieving efficient RNA editing. In the search for better pharmacokinetic properties, it was found earlier that a 2′-O-methoxyethyl (or 2′-methoxyethoxy, or 2′-MOE) modification of the ribose of some, but not all, nucleotides surprisingly appeared compatible with efficient ADAR engagement and editing (WO2019/158475). In a similar fashion, it was found earlier that a PS linkage at some, but not all, internucleoside linkages surprisingly appeared compatible with efficient ADAR engagement and editing (WO2019/219581). Also, it was found earlier that phosphonoacetate linkage modifications and/or unlocked nucleic acid (UNA) ribose modifications of some, but not all, positions in the AON appeared compatible with efficient engagement of an enzyme with nucleotide deamination activity and with subsequent deamination (WO2020/165077). Whereas the properties of phosphonoacetate and UNA modifications were known as such, the compatibility thereof with engagement of enzymes with nucleotide deamination activity and with the deamination reaction was not known.

Disclosed herein are AONs that can provide (mediate, cause, or trigger) RNA editing of a target adenosine in a target transcript molecule, such as pre-mRNA and/or mRNA. The target transcript molecule may be encoded by a mutated gene, wherein the mutation is the cause of a disease and wherein the editing can reverse the mutation to give rise to a wildtype protein, or a protein with a wildtype function (for instance when the mutated amino acid is changed to an amino acid that does not cause the disease, or that provides an improved phenotype). As disclosed in more detail herein, the target transcript molecule may also be encoded by a wildtype gene, such as in a preferred aspect of the present disclosure, wherein the target nucleic acid molecule is a transcript from a wildtype human SLC10A1 gene as shown in the present disclosure, wherein the RNA editing makes that the encoded NTCP protein obtains a loss-of-function, but that improves the disease state of the treated subject.

Non-limiting examples of transcript molecules that are targeted using RNA editing for a variety of treatments are SERPINA1 (for the treatment of alpha1-antitrypsin (A1AT) deficiency; see e.g., WO2016/097212, WO2017/220751, WO2018/041973, and WO2021/243023), IDUA (for the treatment of Hurler syndrome; see e.g., WO2017/220751, WO2018/041973, and WO2021/209010), LRRK2 (for the treatment of Parkinson's disease; WO2016/097212, WO2017/220751, WO2018/041973, WO2021/231673 and WO2021/242903), ABCA4 (for the treatment of Stargardt disease; see e.g., WO2021/130313 and WO2021/231830), USH2A (for the treatment of Usher syndrome; e.g., WO2020/157008, WO2020/219981 and WO2021/136404), APP (e.g., WO2021/113270), CMT1A (e.g., WO2021/113390), ASS1 (e.g., WO2021/231675), GJB2 (e.g., WO2021/231679), MECP2 (for the treatment of Rett syndrome; e.g., WO2019/071274 and WO2021/231680), OTOF (for the treatment of autosomal recessive non-syndromic hearing loss; e.g., WO2021/231685 and WO2021/231692), XLRS (e.g., WO2021/231691), and PCSK9 (for the treatment of hypercholesterolemia; e.g., WO2023/152371).

The present disclosure relates to AONs that mediate RNA editing, using endogenous (naturally present) ADAR enzymes in the host cell, preferably hepatocytes, of one or more adenosines present in the transcript of the SLC10A1 gene. A two-dimensional structure of the NTCP protein in the cell membrane of hepatocytes is known from the art (Ho R H et al. 2004; J Biol Chem. 279 (8): 7213-7222). An AON as disclosed herein aims to reduce the reabsorption of bile acids in the liver by inhibiting NTCP function. Several loss-of-function variants of NTCP have been identified in the art. Hence, these mutations naturally occur in a limited number of people, but do not cause any symptoms associated with cholestasis (Vaz et al. 2015; Hepatology. 61(1): 260-267; Schneider et al. 2022; Clin Res Hepatol Gastroenterol. 46(3): 101824), even though bile acid concentrations in the circulation remain relatively high. Several amino acid substitutions in the sodium-binding pocket leading to loss-of-function mutants of the NTCP protein have been described in the art leading to the inability of bile acid transport without impairing the expression or localization of the protein (Huan Yan et al. 2014; J Virology. 88 (6): 3273-3284). This finding suggests that treating subjects suffering from disorders related to accumulated bile with an AON as disclosed herein will result in the reduction of such accumulation of toxic bile acids in the liver. Moreover, it is anticipated that generating a loss-of-function variant of NTCP will also promote the elimination of bile acids from the body by increasing their excretion in the faeces and urine. This is a process referred to as sulfation of bile acids, which enhances bile acid solubility and reduces their absorption in the intestines. The present disclosure relates to a variety of AONs that are aimed at deamination of a variety of adenosines in the SLC10A1 transcript, of which it is thought that each can independently cause a loss-of-function of the NTCP protein, and each independently can be used to treat the bile acid accumulation disorders as disclosed herein. However, it is not excluded that two or more adenosines may be targeted for deamination in a single treatment. Without wishing to be bound by theory a synergistic or additive effect may be obtained by combining AONs as disclosed herein for targeting a multitude of adenosines, and thereby a multitude of amino acids within a single NTCP protein, to increase the therapeutic effect.

Cholestasis can cause inflammation and lead to the development of liver fibrosis, liver cirrhosis, hepatocellular carcinoma, and/or liver failure. The AONs as disclosed herein are designed to decrease toxic build-up of bile acids to reduce inflammation and lessen or prevent fibrosis and cirrhosis in order to protect liver cells and function.

Causes of cholestasis can be chronic or acute. Chronic liver diseases cause fibrosis and cirrhosis (scarring) of liver tissue that reduces liver function. Chronic conditions can include infectious diseases such as hepatitis virus (hepatitis B virus, hepatitis C virus, hepatitis D virus); alcohol-induced hepatitis; autoimmune and autoinflammatory conditions including primary sclerosing cholangitis, primary biliary cholangitis, and autoimmune hepatitis; metabolic disorders including non-alcoholic fatty liver disease, metabolic-associated fatty liver disease, and non-alcoholic steatohepatitis; inherited disorders including Wilson's disease, hereditary hemochromatosis, and α1-antitrypsin disease. Acute hepatitis and cholestasis can result from infections including hepatitis viruses, mononucleosis, HIV, cytomegalovirus, sepsis, gallbladder infection; from alcohol poisoning or toxic hepatitis; from liver cancer or lymphoma; from use of medications (including birth control pills, anabolic steroids, penicillin antibiotics amoxicillin, including azathioprine, imipramine, estradiol, cimetidine, chlorpromazine, prochlorperazine, tolbutamide, terbinafine); and cholestasis of pregnancy.

Cholestasis can also result from causes outside of the liver from bile duct obstructions or stricture, including gallstones in the common bile duct, cystic duct, or Hartmann's pouch; pancreatic cysts and pseudocysts; extrahepatic bile duct tumors; chronic pancreatitis; pancreatic cancer; bile duct cancer; cholangitis; biliary atresia; and from prior injury or surgery. Furthermore, cholestasis can also occur in neonates, and the causes can include infectious agents such as viruses, bacteria, spirochetes, parasites; toxins from drugs, endotoxins, total parenteral nutrition-associated cholestasis, or herbal products; metabolic causes including hypothyroidism or panhypopituitarism; immune-related gestational alloimmune liver disease; anatomic obstructions including biliary atresia, choledochal cyst, cholelithiasis, biliary sludge, inspissated bile, spontaneous perforation of common bile duct or tumor; cholestasis from idiopathic neonatal hepatitis (transient neonatal cholestasis), cardiovascular and circulatory disorders, hemophagocytic lymphohistiocytosis, malignancy, or congenital lupus; or genetic and metabolic etiologies including alpha1-antitrypsin (A1AT) deficiency, Alagille syndrome, arthrogryposis-renal dysfunction-cholestasis syndrome, Caroli disease, congenital hepatic fibrosis, chromosomal trisomy 21, Turner syndrome, citrin deficiency, cystic fibrosis, disorders of bile acid synthesis, disorders of bile acid conjugation, fatty acid oxidation defects, galactosaemia, glycogen storage disease type IV, hereditary fructose intolerance, mitochondrial respiratory chain disorders, neonatal ichthyosis-sclerosing cholangitis syndrome, neonatal sclerosing cholangitis, Niemann-Pick disease type C, peroxisomal disorders, progressive familial intrahepatic cholestasis, lipid storage diseases, tyrosinaemia, or urea cycle defects. The AONs as disclosed herein are designed to be used against all the disorders above, in all pharmaceutical preparations, uses and methods of treatment as disclosed herein, by preventing bile acid build up in the liver and the resulting cholestasis. The AONs as disclosed herein can also be used to treat infection with Hepatitis B Virus and Hepatitis D virus, by altering the NTCP structure and blocking uptake by hepatocytes.

### Definitions

Whenever reference is made to an oligonucleotide, oligo, ON, ASO, oligonucleotide composition, antisense oligonucleotide, AON, (RNA) editing oligonucleotide, EON, and RNA (antisense) oligonucleotide, both oligoribonucleotides and deoxyoligoribonucleotides are meant unless the context dictates otherwise. Potentially the oligonucleotide may completely lack RNA and DNA nucleotides (as they appear in nature) and may consist completely of modified nucleotides. Whenever reference is made to an ‘oligoribonucleotide’ it may comprise the bases A, G, C, U, or I. Whenever reference is made to a ‘deoxyoligoribonucleotide’ it may comprise the bases A, G, C, T, or I. However, an AON as disclosed herein may comprise a mix of ribonucleotides and deoxyribonucleotides. When a deoxyribonucleotide is used, hence without a modification at the 2′ position of the sugar, the nucleotide is often abbreviated to dA (or Ad), dC (or Cd), dG (or Gd) or T in which the ‘d’ represents the deoxy nature of the nucleoside, while a ribonucleoside that is either normal RNA or modified at the 2′ position is often abbreviated without the ‘d’, and often abbreviated with their respective modifications and as explained herein.

The term ‘nucleoside’ refers to the nucleobase linked to the (deoxy) ribosyl sugar, without phosphate groups. A ‘nucleotide’ is composed of a nucleoside and one or more phosphate groups. The term ‘nucleotide’ thus refers to the respective nucleobase-(deoxy) ribosyl-phospholinker, as well as any chemical modifications of the ribose moiety or the phospho group. Thus, the term would include a nucleotide including a locked ribosyl moiety (comprising a 2′-4′ bridge, comprising a methylene group or any other group), an unlocked nucleic acid (UNA), a threose nucleic acid (TNA), a nucleotide including a linker comprising a phosphodiester, phosphonoacetate, phosphotriester, PS, phosphoro (di)thioate, MP (or MeP), methyl thiophosphonate, phosphoramidate linkages, PNdmi, and a linkage according to the structure of formula (I) as described herein. Sometimes the terms nucleobase, nucleoside and nucleotide are used interchangeably, unless the context clearly requires differently, for instance when a nucleoside is linked to a neighbouring nucleoside and the linkage between these nucleosides is modified. As stated herein, a nucleotide is a nucleoside plus one or more phosphate groups. The terms ‘ribonucleoside’ and ‘deoxyribonucleoside’, or ‘ribose’ and ‘deoxyribose’ are as used in the art.

Sometimes the terms adenosine and adenine, guanosine and guanine, cytidine and cytosine, uracil and uridine, thymine and thymidine/uridine, inosine, and hypoxanthine, are used interchangeably to refer to the corresponding nucleobase on the one hand, and the nucleoside or nucleotide on the other. The nucleobase thymine (T) is also known as 5-methyluracil (m⁵U) and is a uracil (U) derivative; thymine and 5-methyluracil can be interchanged throughout the document text. Likewise, the nucleotide thymidine is also known as 5-methyluridine and is a uridine derivative; thymidine and 5-methyluridine can be interchanged throughout the document text.

Whenever reference is made to nucleotides in the oligonucleotide, such as cytosine, 5-methylcytosine, 5-hydroxymethylcytosine, 5-formylcytosine, 5-acetylcytosine, 5-hydroxycytosine, and β-D-glucosyl-5-hydroxymethylcytosine are included. Whenever reference is made to adenine, N6-methyladenine, 8-oxo-adenine, 2,6-diaminopurine and 7-methyladenine are included. Whenever reference is made to uracil, dihydrouracil, isouracil, N3-glycosylated uracil, pseudouracil, 5-methyluracil, N1-methylpseudouracil, 4-thiouracil and 5-hydroxymethyluracil are included. Whenever reference is made to guanine, 1-methylguanine, 7-methylguanosine, N2, N2-dimethylguanosine, N2,N2,7-trimethylguanosine and N2,7-dimethylguanosine are included. Whenever reference is made to nucleosides or nucleotides, ribofuranose derivatives, such as 2′-deoxy, 2′-hydroxy, and 2′-O-substituted variants, such as 2′-O-methyl(2′-OMe), are included, as well as other modifications, including 2′-4′ bridged variants. Whenever reference is made to oligonucleotides, one or more linkages may be a naturally occurring phosphodieaster linkage, whereas the remaining linkages between two mononucleotides may be a modified linkage. Examples of such modified linkages are phosphonoacetate, phosphotriester, PS, phosphoro (di) thioate, MP, phosphoramidate linkages, phosphoryl guanidine, thiophosphoryl guanidine, sulfono phosphoramidate, PNdmi and the linkage structure according to formula (I), further outlined in detail below.

The term ‘comprising’ encompasses ‘including’ as well as ‘consisting of’, e.g., a composition ‘comprising X’ may consist exclusively of X or may include something additional, e.g., X+Y. The term ‘about’ in relation to a numerical value x is optional and means, e.g., X±10%.

The word ‘substantially’ does not exclude ‘completely’, e.g., a composition which is ‘substantially free from Y’ may be completely free from Y. Where relevant, the word ‘substantially’ may be omitted from the definition of the invention.

The term ‘conducive to’ or ‘mediate’ can be used interchangeably with ‘capable of facilitating’. When used in the context of an AON that is conducive to ADAR editing (or can mediate ADAR editing), this means that the AON, after entry into the cell, interacts with the target RNA sequence, thereby forming a double stranded structure which is recognized by the ADAR enzyme, which can then deaminate the target adenosine into an inosine. Hence, the AON itself does not have the enzymatic function (the ADAR enzyme has), but it can trigger, induce, cause, organize, mediate, provide, give, produce, facilitate, result in RNA editing after binding to the target RNA molecule.

The term ‘mismatch’ is used herein to refer to opposing nucleotides in a double stranded RNA complex which do not form perfect base pairs according to the Watson-Crick base pairing rules. In the historical sense, mismatched nucleotides are G-A, C-A, U-C, A-A, G-G, C-C, U-U pairs. In some embodiments AONs as disclosed herein comprise fewer than four mismatches with the target sequence, for example 0, 1 or 2 mismatches. ‘Wobble’ base pairs are G-U, I-U, I-A, and I-C base pairs. When a U is placed opposite the target A, there is no mismatch, and the AON may be 100% complementary. When a C is placed opposite the target A, there is at least 1 mismatch between the AON and the target sequence. Although a G: G pairing would be considered a mismatch, that does not necessarily mean that the interaction is unstable, which means that the term ‘mismatch’ may be somewhat outdated based on the current disclosure where a Hoogsteen base-pairing may be seen as a mismatch based on the origin of the nucleotide but still be relatively stable. An isolated G: G pairing in duplex RNA can for instance be quite stable, but still be defined as a mismatch. Analysis of natural targets of ADAR enzymes has indicated that these generally include mismatches between the two strands that form the RNA helix edited by ADAR1 or 2. It has been suggested that these mismatches enhance the specificity of the editing reaction (Stefl et al. 2006. Structure 14 (2): 345-355; Tian et al. 2011. Nucleic Acids Res 39 (13): 5669-5681). Characterization of optimal patterns of paired/mismatched nucleotides between the AONs and the target RNA also appears important to the development of efficient ADAR-based AON therapy.

The term ‘complementary’ as used herein refers to the fact that the AON hybridizes under physiological conditions to a second nucleic acid strand. Examples are (i) when the AON as a first nucleic acid strand (=guide oligonucleotide) forms a heteroduplex RNA editing oligonucleotide complex with second complementary nucleic acid strand (in vitro), or (ii) when it forms a double stranded complex with the target RNA molecule. The term does not necessarily mean that each nucleotide in a nucleic acid strand has a perfect pairing with its opposite nucleotide in the opposite sequence. In other words, while an AON may be complementary to a target sequence, there may be mismatches, wobbles and/or bulges between the AON and the target sequence, while under physiological conditions that AON still hybridizes to the target sequence such that the cellular RNA editing enzymes can deaminate the target adenosine to an inosine. The term ‘substantially complementary’ therefore also means that despite the presence of the mismatches, wobbles, and/or bulges, the AON has enough matching nucleotides with the target sequence that under physiological conditions the AON hybridizes to the target RNA molecule. As shown herein, an AON may be complementary, but may also comprise one or more mismatches, wobbles and/or bulges with the target sequence, if under physiological conditions the AON is able to hybridize to its target.

The term ‘orphan nucleotide’ relates to the nucleotide in the AON that is directly opposite the target adenosine, which is the adenosine that is deaminated by the deaminating enzyme. The orphan nucleotide may be a natural cytidine or deoxycytidine, or a uridine or deoxyuridine. It may also be a chemically modified nucleotide, as further described in detail below, or a known or chemically modified analog of a natural (deoxy) cytidine, such as a nucleotide carrying a Benner's base, or a known or chemically modified analog of a natural (deoxy) uridine, such as iso-uridine, as further outlined in detail below.

A ‘nucleotide analog’ refers to an analog of a nucleic acid nucleotide. The nucleotide analog is an analog of adenosine, guanosine, cytidine, thymidine, uridine, deoxyadenosine, deoxyguanosine, deoxycytidine, deoxythymidine or deoxyuridine.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand. The same holds true for the AONs as disclosed herein. Nucleotides that are upstream of the orphan nucleotide in the antisense oligonucleotide are located towards the 5′ terminus, and nucleotides that are downstream of the orphan nucleotide are located towards the 3′ terminus.

The nucleotide ‘numbering’ in an AON as disclosed herein is such that the orphan nucleotide is number 0 and the nucleotide 5′ from the orphan nucleotide is number +1. Counting is further positively (+) incremented towards the 5′ end and negatively (−) incremented towards the 3′ end, wherein the first nucleotide 3′ from the orphan nucleotide is number −1. The internucleoside linkage numbering in the AON is such that linkage number 0 is the linkage 5′ from the orphan nucleotide, and the linkage positions in the oligonucleotide are positively (+) incremented towards the 5′ end and negatively (−) incremented towards the 3′ end.

References to ‘hybridisation’ typically refer to specific hybridisation and exclude non-specific hybridisation. Specific hybridisation can occur under experimental conditions chosen, using techniques well known in the art, to ensure that most stable interactions between probe and target are where the probe and target have at least 70%, preferably at least 80%, more preferably at least 90% sequence identity.

The term ‘splice mutation’ relates to a mutation in a gene that encodes for a pre-mRNA, wherein the splicing machinery is dysfunctional in the sense that splicing of introns from exons is disturbed and due to the aberrant splicing, the subsequent translation is out of frame resulting in premature termination of the encoded protein. Often such shortened proteins are degraded rapidly and do not have any functional activity.

Whenever a ‘naked’ form in relation to the AON as disclosed herein is referred to, it means that the AON is manufactured in a laboratory or manufacturing facility, through which it is generally chemically modified to prevent it from rapid degradation after it enters the mammalian body or a tissue, or cell, upon administration. A naked form of an AON is therefore different from a form in which the AON is encoded (and delivered) by a viral genome or within a plasmid vector. When such viral vectors or plasmid vectors are administered, the encoded AON is expressed from the viral vector genome or from the plasmid in the cell to which the viral vector or plasmid vector is delivered. Consequently, the AON is then not chemically modified, and comprises solely naturally occurring RNA nucleotides.

The length of the AON as disclosed herein, and when delivered in a naked form is preferably 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length. However, when the AON as disclosed herein is to be delivered through the expression of a viral vector, then the AON may be longer, such as 70, 80, 90, 100, 150, or 200 or more nucleotides in length.

The term ‘HEON’ refers to a heteroduplex double-stranded complex molecule wherein an AON as disclosed herein is hybridized to a partially or fully complementary, partially of fully overlapping sense oligonucleotide. Because the AON as disclosed herein often has specified chemical modifications that are different from the chemical modifications in the sense strand, the two strands form such a heteroduplex RNA editing oligonucleotide complex. The sense strand may be chemically modified almost in its entirety, similar or different to what is performed in the AON as disclosed herein, for example by providing nucleotides with a ribose sugar moiety carrying a 2′-OMe substitution, a 2′-F substitution, or a 2′-MOE substitution. It is to be understood that the sense strand present in the HEON is a different entity in comparison to the target RNA molecule in the cell. The sense strand in an HEON is preferably 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length. The HEON is often generated in vitro and used as a delivery tool to protect the AON from degradation when administered to the cell. In other words, the HEON is preferably formed before the AON is administered to the cell.

Disclosed herein is an AON that is capable of recruiting an endogenous ADAR enzyme in a human cell after the AON has formed a double-stranded complex with a region of a target RNA nucleic acid molecule in a cell, wherein the region comprises a target adenosine, wherein the nucleotide in the AON that is opposite the target adenosine is the orphan nucleotide, wherein the ADAR enzyme can deaminate the target adenosine into an inosine after binding to the double-stranded complex, and wherein the target RNA nucleic acid molecule is a transcript molecule of the human SLC10A1 gene encoding NTCP. Preferably, the transcript molecule is a pre-mRNA or an mRNA molecule. Preferably, the cell is a liver cell, more preferably a hepatocyte. In one aspect, the nucleotide numbering within the AON is such that the orphan nucleotide is number 0 and nucleotides are further positively (+) incremented towards the 5′ end and negatively (−) incremented towards the 3′ end, and wherein the orphan nucleotide is a deoxynucleotide comprising a cytosine, a cytosine analog, a uracil, or an iso-uracil. In one aspect, the orphan nucleotide is a deoxynucleotide comprising a cytosine analog, wherein the cytosine analog is a 6-amino-5-nitro-3-yl-2 (1H)-pyridone nucleobase, also referred to as a Benner's base. In certain aspects of the AONs as disclosed herein, the first nucleotide 3′ from the orphan nucleotide is a deoxyinosine when the nucleotide opposite this position is a cytidine in the target RNA nucleic acid molecule. Such is the case when the target adenosine is a) in the codon for glutamine at position 68, b) is the first adenosine in the codon for glutamine at position 261, or c) is the first adenosine in the codon for lysine at position 314. In one aspect, the AON is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length. In one aspect, the AON comprises one or more modifications in the linkage moiety, which is each independently selected from the group consisting of: PS, phosphonoacetate, phosphorodithioate, MP, sulfonylphosphoramidate, PNdmi, and PNms. In one aspect, the internucleoside linkage numbering in the AON is such that linkage number 0 is the linkage 5′ from the orphan nucleotide, and the linkage positions in the oligonucleotide are positively (+) incremented towards the 5′ end and negatively (−) incremented towards the 3′ end, and wherein linkage position-2 is an MP linkage or a PNms linkage. In one aspect, the linkage between the most terminal two nucleotides on the 5′ and/or 3′ terminus of the AON is a PNdmi linkage or a PNms linkage. In one aspect, the AON comprises one or more nucleotides comprising a mono- or di-substitution at the 2′, 3′ and/or 5′ position of the ribose, each independently selected from the group consisting of: —OH; —F; substituted or unsubstituted, linear or branched lower (C₁-C₁₀)alkyl, alkenyl, alkynyl, alkaryl, allyl, or aralkyl, that may be interrupted by one or more heteroatoms; -O—, S—, or N-alkyl; -O—, S-, or N-alkenyl; -O—, S—, or N-alkynyl; -O—, S—, or N-allyl; -O-alkyl-O-alkyl; -methoxy; -aminopropoxy; -methoxyethoxy; -dimethylamino oxyethoxy; and -dimethylaminoethoxyethoxy. In one aspect, the AON is covalently or non-covalently, directly or through a linker, bound to a GalNAc moiety. The skilled person can select the right linker and need for covalent or non-covalent binding of a GalNAc moiety, when the AON needs to be delivered to liver cells, especially hepatocytes. In one aspect, the AON is covalently or non-covalently, directly or through a linker, bound to a triterpene glycoside, preferably AG1856. As described in PCT/EP2024/051278 (unpublished), it is very efficient to increase RNA editing, when an AON is connected 1:1 with a saponin, especially when the saponin is AG1856. Hence, to increase the endosomal release (intracellularly) of the AON and make it available for RNA target hybridization, it is preferred that the AON is attached (covalently, or non-covalently) to AG1856 before administration to the cell or the subject to be treated.

Disclosed herein is an AON that is capable of recruiting an endogenous ADAR enzyme in a human cell after the AON has formed a double-stranded complex with a region of a target RNA nucleic acid molecule in a cell, wherein the region comprises a target adenosine, wherein the nucleotide in the AON that is opposite the target adenosine is the orphan nucleotide, wherein the ADAR enzyme can deaminate the target adenosine into an inosine after binding to the double-stranded complex, wherein the SLC10A1 gene is wildtype, and wherein the target adenosine is selected from the group consisting of: (i) the adenosine in the CAG codon coding for glutamine (Q) at position 68 of the NTCP protein, and wherein the deamination of the adenosine changes the amino acid to an arginine (R); (ii) the first adenosine in the CAA codon coding for glutamine (Q) at position 261 of the NTCP protein, and wherein the deamination of the adenosine changes the amino acid to an arginine (R); (iii) the adenosine in the GAG codon coding for glutamic acid (E) at position 257 of the NTCP protein, and wherein the deamination of the adenosine changes the amino acid to glycine (G); and (iv) the first adenosine in the AAG codon coding for lysine (K) at position 314 of the NTCP protein, and wherein the deamination of the adenosine changes the amino acid to glutamic acid (E); wherein the deamination of the target adenosine results in an NTCP protein that is impaired in its function to transport bile acids from portal circulation into the cell. In a preferred aspect, the target adenosine is in the CAG codon coding for glutamine at position 68 of the NTCP protein, and wherein the AON comprises or consists of the sequence and modifications selected from the group consisting of SEQ ID NO: 150, 151, 152, 154, 156, 158, 159, 163, 164, 165, 166, 1127, 1128, 1129, 1130, 1131, 1133, 1137, 1138, 1139, 1140, 1141, 1142, 1143, 1144, 1145, 1146, 1150, 1151, 1152, 1153, 1154, 1155, 1156, 1157, 1158, 1159, 1160, 1161, 1167, 1168, 1169, 1170, 1171, 1172, 1173, 1174, 1175, 1176, 1177, 1178, 1180, 1181, 1182, 1183, 1260, 1283, 1284, 1285, 1286, 1287, 1288, 1295, 1296, 1297, 1298, 1299, 1300, 1301, 1302, 1303, 1304, 1305, and 1306. In another preferred aspect, the target adenosine is in the GAG codon coding for glutamic acid at position 257 of the NTCP protein, and wherein the AON comprises or consists of the sequence and modifications selected from the group consisting of SEQ ID NO:1193, 1194, 1197, 1198, 1199, 1200, 1201, 1202, 1203, 1204, 1205, 1206, 1207, 1208, 1209, 1210, 1211, 1212, 1213, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1253, and 1254. The present disclosure also relates to a vector, preferably a viral vector, more preferably an adeno-associated virus (AAV) vector, comprising a nucleic acid molecule encoding an AON as disclosed herein. When an encoding vector is applied, and the AON is encoded by the viral vector (genome), the orphan nucleotide is a cytidine, and the nucleotide on position-1 is a guanosine, when the opposing nucleotide in the target sequence is a cytidine. Furthermore, since the AON is transcribed from the viral vector (genome), no chemical modifications are present in the backbone, linkages, and sugar ribose. The present disclosure also relates to a nanoparticle delivery vehicle formulation comprising an AON as disclosed herein. In a preferred aspect, the nanoparticle delivery vehicle is a lipid nanoparticle (LNP). LNP's that can be used in the context of the AON of the present disclosure are those that have been used in the art for the delivery of small and large RNA molecules, such as those applied in the delivery of mRNA-based vaccines like those against Covid-19 coronaviruses. LNP's that have been applied for the delivery of other types of RNA, such as siRNA can also be applied for the delivery of the AON as disclosed herein. If an LNP is applied or any other similar type of carrier, the AON is still considered naked because it is not transcribed from an encoding polynucleotide (such as in the case of a plasmid or a vector, in which the AON is not regarded as ‘naked’). So, even though a chemically modified AON is encapsulated by a carrier, preferably an LNP, it is still seen as naked, as it has been manufactured as such in a laboratory setting and encapsulated thereafter in the carrier using methods known to the person skilled in the art. The disclosure also relates to a delivery vehicle, preferably an LNP, which comprises a ‘naked’ and chemically modified AON as disclosed herein, even more preferably as disclosed in any one of SEQ ID NO: 150, 151, 152, 154, 156, 158, 159, 163, 164, 165, 166, 1127, 1128, 1129, 1130, 1131, 1133, 1137, 1138, 1139, 1140, 1141, 1142, 1143, 1144, 1145, 1146, 1150, 1151, 1152, 1153, 1154, 1155, 1156, 1157, 1158, 1159, 1160, 1161, 1167, 1168, 1169, 1170, 1171, 1172, 1173, 1174, 1175, 1176, 1177, 1178, 1180, 1181, 1182, 1183, 1260, 1283, 1284, 1285, 1286, 1287, 1288, 1295, 1296, 1297, 1298, 1299, 1300, 1301, 1302, 1303, 1304, 1305, 1306, 1193, 1194, 1197, 1198, 1199, 1200, 1201, 1202, 1203, 1204, 1205, 1206, 1207, 1208, 1209, 1210, 1211, 1212, 1213, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1253, and 1254. The present disclosure also relates to a pharmaceutical composition comprising an AON, a vector, or a nanoparticle delivery vehicle formulation as disclosed herein, and a pharmaceutically acceptable carrier. In one aspect, an AON as disclosed herein is in a naked form. In one aspect, an AON as disclosed herein is in a circular format. In one aspect, an AON as disclosed herein is not in a naked form but is expressed from the genome of a viral vector. In one aspect, an AON as disclosed herein is not in a naked form but is expressed from an expression vector such as a plasmid. In one aspect, when the AON as disclosed herein is not in a naked form, the AON is 15 to 60 nucleotides in length as indicated above, or in another embodiment, from 61 to 300 nucleotides in length. It should be noted that when an AON is delivered through a vector, for instance an AAV vector, chemical modifications are not present in the AON that acts on the target RNA molecule. Although it is preferred to use ‘naked’ AONs that have chemical modifications as outlined herein, AONs that are delivered through other means, for instance through AAV vector expression, or editing molecules that are circular, or have hairpin structures (recruiting portions, e.g., as disclosed in WO2016/097212, WO2017/050306, WO2020/001793, WO2017/010556, WO2020/246560, and WO2022/078995) are also encompassed by the present disclosure because these can also be applied to edit adenosines in the target SLC10A1 RNA molecule to generate an NTCP protein with reduced function. The person skilled in the art understands that when a delivery moiety, or attachment to the AON is used (such a GalNAc moiety to target hepatocytes in the liver) that the AON is still seen as naked as well, also when a GalNAc-AON is encapsulated in a delivery vehicle such as an LNP. The present disclosure also relates to an AON as disclosed herein, for use in the treatment of a disease caused by bile accumulation in the liver, such as cholestasis, primary sclerosing cholangitis (PSC), biliary atresia (BA), and liver cirrhosis. The present disclosure also relates to a use of an AON, as disclosed herein, in the manufacture of a medicament for the treatment of a disease caused by bile accumulation in the liver, such as cholestasis, PSC, BA, and liver cirrhosis. The present disclosure also relates to a method of editing a human SLC10A1 pre-mRNA or mRNA molecule in a liver cell, preferably a hepatocyte, the method comprising contacting the SLC10A1 pre-mRNA or mRNA molecule with an AON capable of triggering an ADAR-mediated adenosine to inosine deamination, thereby editing the SLC10A1 pre-mRNA or mRNA molecule to encode an NTCP protein with a diminished, lowered, or loss of function in bile acid uptake, and wherein the AON is as disclosed herein. The present disclosure also relates to a method of treating, ameliorating, or slowing down the progression of a disease caused by bile accumulation in the liver, such as cholestasis, PSC, BA, and liver cirrhosis, in a human subject in need thereof, the method comprising administering to said subject an AON, a vector, or a nanoparticle delivery vehicle formulation as disclosed herein, thereby allowing the AON to hybridize to a complementary part of a region comprising a target adenosine in a SLC10A1 pre-mRNA or mRNA molecule in a cell of the subject, thereby effecting an ADAR-mediated adenosine to inosine deamination of the target adenosine, thereby changing the SLC10A1 pre-mRNA or mRNA molecule such that it encodes an NTCP protein with a diminished, lowered, or loss of function in bile acid uptake, thereby treating the subject. The RNA editing can in principle be forced to take place at a variety of positions in the SLC10A1 transcript. The examples of target adenosines are as disclosed herein, with the adenosine in the CAG codon coding for glutamine (Q) at position 68, the first adenosine in the CAA codon coding for glutamine (Q) at position 261, the adenosine in the GAG codon coding for glutamic acid (E) at position 257, and the first adenosine in the AAG codon coding for lysine (K) at position 314 of the NTCP protein being preferred. Highly preferred are AONs as disclosed herein, and methods of use of these AONs that bring about the Q68R change and/or the E257G change within the human NTCP protein. Also disclosed is an in vitro, ex vivo, or in vivo method for the deamination of a target adenosine in a human SLC10A1 pre-mRNA or mRNA molecule in a liver cell, preferably a hepatocyte, the method comprising the steps of: (i) providing the cell with an AON as disclosed herein; (ii) allowing uptake by the cell of the AON; (iii) allowing annealing of the AON to the SLC10A1 pre-mRNA or mRNA molecule; (iv) allowing an endogenous ADAR enzyme to deaminate the target adenosine in the SLC10A1 pre-mRNA or mRNA molecule to an inosine; and optionally (v) using a functional read-out to identify the presence of the inosine in the SLC10A1 pre-mRNA or mRNA molecule. In one aspect, a method as disclosed herein comprises the step of administering a triterpene glycoside before, after or simultaneously with administering the AON, wherein in a preferred aspect, the triterpene glycoside is AG1856. However, in a preferred aspect the triterpene glycoside (or ‘saponin’ as it is often referred to) is physically bound to the AON. In one aspect, an AON as disclosed herein comprises a linkage moiety with the structure according to formula (I)

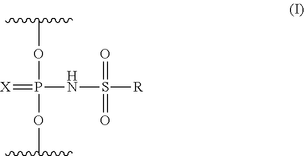

wherein: X═O or S; and R=an aryl, a substituted aryl, a heterocycle, a substituted heterocycle, an aromatic heterocycle, a substituted aromatic heterocycle, a C₁-C₆ alkoxy, a substituted C₁-C₆ alkoxy, a C₁-C₂₀ alkyl, a substituted C₁-C₂₀ alkyl, a C₁-C₆ alkenyl, a C₁-C₆ substituted alkenyl, a C₁-C₆ alkynyl, a substituted C₁-C₆ alkynyl, or a conjugate group. In a preferred embodiment, X═O and R=methyl and the linkage modification is referred to as mesyl phosphoramidate, MsPA or “PNms”. In one embodiment, a PNms linkage is used instead of the MP and/or PNdmi linkages.

### Chemical Modifications

Various chemistries and modifications are known in the field of oligonucleotides that can be readily used in accordance with the invention. All chemical modifications listed herein that may be used in the AON as disclosed herein may also be used for a sense strand that is complementary to the AON, when the AON and the complementary strand form a HEON complex, such as described in GB 2215614.5 (unpublished) and as disclosed above, except that the opposite sense strand does not have an orphan nucleotide. Hence, the modification related to the orphan nucleotide relate only to the AON as disclosed herein, but all other modifications relate to the AON as disclosed herein and any (protecting) sense oligonucleotide that may be used together with the AON in a pharmaceutical product. This includes the use of hydrophobic moieties (such as tocopherol and cholesterol) and cell-specific ligands (such as GalNAc moieties), that have also been described herein, and in detail in PCT/EP2023/079290 (unpublished), which may either be bound to the AON or its opposite strand, or both. Preferred GalNAc moieties that can be used in the context of the AONs as disclosed herein are disclosed in WO2022/271806.

The skilled person knows that an oligonucleotide, such as an AON as outlined herein, generally consists of repeating monomers. Such a monomer is most often a nucleotide or a chemically modified nucleotide. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide. A modification in the pentose sugar is therefore often referred to as a ‘scaffold modification’. The original pentose sugar may be replaced in its entirety by another moiety that similarly connects the base and the phosphate. It is therefore understood that while a pentose sugar is often a scaffold, a scaffold is not necessarily a pentose sugar. Examples of scaffold modifications that may be applied in the monomers of the AON as disclosed herein are disclosed in WO2020/154342, WO2020/154343, and WO2020/154344.

A nucleoside in the AON as disclosed herein may be a natural nucleoside (deoxyribonucleoside or ribonucleoside) or a non-natural nucleoside. It is noted that for RNA editing, in which double-stranded RNA is generally the substrate for enzymes with deamination activity (such as ADARs), ribonucleosides are considered ‘natural’, while deoxyribonucleosides may then be, for the sake of argument, considered as non-natural, or modified, simply because DNA is not present in the RNA-RNA double stranded (natural) substrate configurations. The skilled person appreciates that when the nucleotide has a natural ribose moiety, it may still be non-naturally modified in the base and/or the linkage.

It is recognized in the art that common limiting factors in oligonucleotide-based therapies are the oligonucleotide's ability to be taken up by the cell (when delivered per se, or ‘naked’ without applying a delivery vehicle such as a viral vector or plasmid), the biodistribution and the resistance to nuclease-mediated breakdown. The skilled person is aware, and it has been described in detail in the art, that a variety of chemical modifications can assist in overcoming such limitations. Examples of such now commonly used chemical modifications are the 2′-OMe, 2′-F and 2′-MOE modifications of the sugar and the use of PS linkages between nucleosides, as described herein.

### Scaffold Modifications (Ribose)

The ribose 2′ groups in all nucleotides of the AON as disclosed herein, except for the ribose sugar moiety of the orphan nucleotide that has certain limitations in respect of compatibility with RNA editing, can be independently selected from 2′-H (i.e., DNA), 2′-OH (i.e., RNA), 2′-OMe, 2′-MOE, 2′-F, or 2′-4′-linked (for instance a locked nucleic acid (LNA)), or other ribosyl 1′-substitutions, 2′ substitutions, 3′ substitutions, 4′ substitutions or 5′ substitutions. The orphan nucleotide in the AON that comprises no other chemical modifications to the ribose sugar, the base, or the linkage preferably does not carry a 2′-OMe or 2′-MOE substitution when the nucleobase is a naturally occurring cytosine, but may carry a 2′-F, a 2′,2′-difluoro (diF), or 2′-ara-F (FANA) substitution or may be DNA. WO2024/013360 discloses the modification of the 2′ position of the ribose sugar moiety of the orphan nucleotide by a 2′,2′-disubstituted substitution such as diF, which is also applicable to what is disclosed here. The 2′-4′ linkage can be selected from many linkers known in the art, such as a methylene linker, amide linker, or constrained ethyl linker (cEt).

An AON as disclosed herein may comprise one or more nucleotides carrying a 2′-MOE ribose modification. Also, an AON as disclosed herein may comprise one or more nucleotides not carrying a 2′-MOE ribose modification, or wherein the 2′-MOE ribose modifications are at positions that do not prevent the enzyme with adenosine deaminase activity from deaminating the target adenosine. An AON as disclosed herein may comprise a 2′-OMe ribose modification at a position that does not comprise a 2′-MOE ribose modification. An AON as disclosed herein may comprise deoxynucleotides at positions that do not comprise a 2′-MOE or a 2′-OMe ribose modification, or other 2′ ribose substitution. An AON as disclosed herein may comprise one or more nucleotides comprising a 2′ substitution comprising a 2′-MOE, 2′-OMe, 2′-OH, 2′-deoxy, TNA, 2′-fluoro (2′-F), 2′,2′-difluoro (diF) modification, 2′-fluoro-2′-C-methyl modification, or a 2′-4′-linkage (i.e., a bridged nucleic acid such as a locked nucleic acid (LNA or examples mentioned in e.g. WO2018/007475)). Other nucleic acid monomers that may be used in an AON as disclosed herein are arabinonucleic acids and 2′-deoxy-2′-fluoroarabinonucleic acid (FANA), for instance for improved affinity purposes. The 2′-4′ linkage can be selected from linkers known in the art, such as a methylene linker or constrained ethyl linker. A wide variety of 2′ modifications that may present in an AON as disclosed herein are known in the art, including but not limited to the modifications outlined in detail in WO2016/097212, WO2017/220751, WO2018/041973, WO2018/134301, WO2019/219581, WO2019/158475, and WO2022/099159. In all cases, the modifications should be compatible with RNA editing such that the AON fulfils its role as an oligonucleotide that can form a double stranded complex with the target RNA and by generating this double-stranded nucleic acid complex, recruit a deaminating enzyme, which can subsequently deaminate the target adenosine. Where a monomer in an AON as disclosed herein comprises an unlocked nucleic acid (UNA) ribose modification, that monomer can have a 2′ position comprising the same modifications discussed above, such as a 2′-MOE, a 2′-OMe, a 2′-OH, a 2′-deoxy, a 2′-F, a 2′,2′-diF, a 2′-fluoro-2′-C-methyl, an arabinonucleic acid, a FANA, or a 2′-4′-linkage (i.e., a bridged nucleic acids such as a locked nucleic acid (LNA)). In an aspect, the AON as disclosed herein comprises at least one nucleotide comprising a threose nucleic acid (TNA) ribose modification. In one aspect, the AON as disclosed herein comprises at least one nucleotide with a sugar moiety that comprises a 2′-fluoro (2′-F) modification. A preferred position for the nucleotide that carries a 2′-F modification is position-3 in AON, which may be present together with an identical 2′ modification in the orphan nucleotide as discussed above.

### Base Modifications

A base, sometimes called a nucleobase, is generally adenine, cytosine, guanine, thymine or uracil, or a derivative thereof. A nucleobase is defined as a moiety that can bond to another nucleobase through H-bonds, polarized bonds (such as through CF moieties) or aromatic electronic interactions. Cytosine, thymine, and uracil are pyrimidine bases, and are generally linked to the scaffold through their 1-nitrogen. Adenine and guanine are purine bases and are generally linked to the scaffold through their 9-nitrogen. The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy) ribosyl sugar. The nucleobases in an AON as disclosed herein can be adenine, cytosine, guanine, thymine, or uracil or any other moiety able to interact with another nucleobase through H-bonds, polarized bonds (such as CF) or aromatic electronic interactions. The nucleobases at any position in the AON as disclosed herein can be a modified form of adenine, cytosine, guanine, or uracil, such as hypoxanthine (the nucleobase in inosine), pseudouracil, pseudocytosine, isouracil, N3-glycosylated uracil, 1-methylpseudouracil, orotic acid, agmatidine, lysidine, 2-thiouracil, 2-thiothymine, 5-substituted pyrimidine (e.g., 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine), 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, 8-oxo-adenine, 3-deazapurine (such as a 3-deaza-adenosine), pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp and its derivatives, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose). Modified bases comprise synthetic and natural bases such as inosine, xanthine, hypoxanthine and other-aza, deaza, -hydroxy, -halo, -thio, thiol, -alkyl, -alkenyl, -alkynyl, thioalkyl derivatives of pyrimidine and purine bases that are or will be known in the art. Purine nucleobases and/or pyrimidine nucleobases may be modified to alter their properties, for example by amination or deamination of the heterocyclic rings. The exact chemistries and formats may vary from oligonucleotide construct to oligonucleotide construct and from application to application, and may be worked out in accordance with the wishes and preferences of those of skill in the art.

A scaffold modification indicates the presence of a modified version of the ribosyl moiety as naturally occurring in RNA (i.e., the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted)alkyl such as 2′-OMe, 2′-O-(2-cyanoethyl), 2′-MOE, 2′-O-(2-thiomethyl)ethyl, 2′-O-butyryl, 2′-O-propargyl, 2′-O-allyl, 2′-O-(2-aminopropyl), 2′-O-(2-(dimethylamino) propyl), 2′-O-(2-amino)ethyl, 2′-O-(2-(dimethylamino)ethyl); 2′-deoxy (DNA); 2′-O-(haloalkyl) methyl such as 2′-O-(2-chloroethoxy) methyl (MCEM), 2′-O-(2,2-dichloroethoxy) methyl (DCEM); 2′-O-alkoxycarbonyl such as 2′-O-[2-(methoxycarbonyl)ethyl] (MOCE), 2′-O-[2-N-methylcarbamoyl)ethyl] (MCE), 2′-O-[2-(N,N-dimethylcarbamoyl)ethyl] (DCME); 2′-halo e.g. 2′-F, FANA; 2′-O-[2-(methylamino)-2-oxoethyl] (NMA); a bicyclic or bridged nucleic acid (BNA) scaffold modification such as a conformationally restricted nucleotide (CRN) monomer, a locked nucleic acid (LNA) monomer, a xylo-LNA monomer, an α-LNA monomer, an α-I-LNA monomer, a β-d-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-O,4′-C) constrained ethyl (cEt) BNA monomer, a (2′-O,4′-C) constrained methoxyethyl (cMOE) BNA monomer, a 2′,4′-BNANC (NH) monomer, a 2′,4′-BNANC (NMe) monomer, a 2′,4′-BNA^{NC} (NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-I-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above. All these modifications are known to the person skilled in the art.

### The Orphan Nucleotide

Mutagenesis studies of human ADAR2 revealed that a single mutation at residue 488 from glutamate to glutamine (E488Q), gave an increase in the rate constant of deamination by 60-fold when compared to the wild-type enzyme (Kuttan and Bass. Proc Natl Acad Sci USA 2012. 109 (48): 3295-3304). During the deamination reaction, ADAR flips the edited base out of its RNA duplex, and into the enzyme active site (Matthews et al. Nat Struct Mol Biol. 2016. 23 (5): 426-433). When ADAR2 edits adenosines in the preferred context (an A: C mismatch) the nucleotide opposite the target adenosine is often referred to as the ‘orphan nucleotide’ (or ‘orphan cytidine’ as the case may be), as indicated above. The crystal structure of ADAR2 E488Q bound to double stranded RNA (dsRNA) revealed that the glutamine (Gln; Q) side chain at position 488 can donate an H-bond to the N3 position of the orphan cytidine, which leads to the increased catalytic rate of ADAR2 E488Q. In the wild-type enzyme, wherein a glutamate (or glutamic acid; Glu; E) is present at position 488 instead of a glutamine (Gln) the amide group of the glutamine is absent and is instead a carboxylic acid. To obtain the same contact of the orphan cytidine with the E488Q mutant would then, for the wild-type situation, require protonation for this contact to occur. To make use of endogenously expressed ADAR2 to correct disease relevant mutations, it is essential to maximize the editing efficiency of the wild type ADAR2 enzyme present in the cell. WO2020/252376 discloses the use of AONs with modified RNA bases, especially at the position of the orphan cytidine to mimic the hydrogen-bonding pattern observed by the E488Q ADAR2 mutant. By replacing the nucleotide opposite the target adenosine in the AON with cytidine analogs that serve as H-bond donors at N3, it was envisioned that it would be possible to stabilize the same contact that is believed to provide the increase in catalytic rate for the mutant enzyme. Two cytidine analogs were of particular interest: pseudoisocytidine (also referred to as ‘piC’; Lu et al. J Org Chem 2009. 74(21):8021-8030; Burchenal et al. (1976) Cancer Res 36:1520-1523) and Benner's base Z (also referred to as ‘dZ’; Yang et al. Nucleic Acid Res 2006. 34 (21): 6095-6101) that were initially selected because they offer hydrogen-bond donation at N3 with minimal perturbation to the shape of the nucleobase. Benner's base is also referred to as a 6-amino-5-nitro-3-yl-2 (1H)-pyridone nucleobase. The presence of the cytidine analog in the AON may exist in addition to modifications to the ribose 2′ group. The ribose 2′ groups in the orphan nucleotide can be independently selected from 2′-H (i.e., DNA), 2′-OH (i.e., RNA), 2′-OMe, 2′-MOE, 2′-F, or 2′-4′-linked (i.e., a bridged nucleic acid such as a locked nucleic acid (LNA)), or other 2′ substitutions. The 2′-4′ linkage can be selected from linkers known in the art, such as a methylene linker or constrained ethyl linker.

The orphan nucleotide in the AON as disclosed herein is preferably a cytidine or analog thereof (such as a nucleotide carrying a Benner's base) or a uridine or analog thereof (such as iso-uridine). The orphan nucleotide, whether it is a cytidine or analog thereof, or a uridine or analog thereof, preferably comprises a deoxyribose (2′-H; =DNA) but may also comprise a diF modification at the 2′ position of the sugar. In an embodiment at least one and in another embodiment both the neighbouring (directly adjacent) nucleotides flanking the orphan nucleotide do not comprise a 2′-OMe modification. Complete modification wherein all nucleotides of the oligonucleotide hold a 2′-OMe modification (including the orphan nucleotide), with natural bases, results in a non-functional oligonucleotide as far as RNA editing goes (known in the art), presumably because it hinders the ADAR activity at the targeted position. In general, an adenosine in a target RNA can be protected from editing by providing an opposing nucleotide with a 2′-OMe group (at least when there are no other chemical substitutions or modifications within the nucleotide), or by providing a guanine or adenine as opposing base, as these two nucleobases are also able to reduce editing of the opposing adenosine.

### Linkage Modifications

A nucleoside is generally connected to neighboring nucleosides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked scaffolds of an oligonucleotide is often called the ‘backbone’ of the oligonucleotide. Because phosphodiester bonds connect neighboring monomers together, they are often referred to as ‘backbone linkages’. It is understood that when a phosphate group is modified so that it is instead an analogous moiety such as a phosphorothioate, such a moiety is still referred to as the backbone linkage of the monomer. This is referred to as a ‘backbone linkage modification’. In general terms, the backbone of an oligonucleotide comprises alternating scaffolds and backbone linkages.

As outlined in detail herein, naked AONs as disclosed herein comprise at least one, preferably multiple linkage modifications. It is generally more preferred that the AON as disclosed herein comprises linkage modifications at most, and potentially all positions if the AON is capable of mediating RNA editing through the deamination enzyme when the AON is bound to the target RNA nucleic acid molecule. A linkage modification can be, but is not limited to, a modified version of the phosphodiester present in RNA, such as phosphorothioate (PS), chirally pure PS, (R)-PS, (S)-PS, methyl phosphonate (MP or MeP), chirally pure MP, (R)-MP, (S)-MP, phosphoryl guanidine (such as PNdmi), chirally pure phosphoryl guanidine, (R)-phosphoryl guanidine, (S)-phosphoryl guanidine, phosphorodithioate (PS2), phosphonacetate (PACE), phosphonoacetamide (PACA), thiophosphonoacetate, thiophosphonoacetamide, methyl phosphorohioate, methyl thiophosphonate, PS prodrug, alkylated PS, H-phosphonate, ethyl phosphate, ethyl PS, boranophosphate, borano PS, methyl boranophosphate, methyl borano PS, methyl boranophosphonate, methyl boranophosphothioate, phosphate, phosphotriester, aminoalkylphosphotriester, and their derivatives. Another modification includes phosphoramidite, phosphoramidate, N3′→P5′ phosphoramidate, phosphorodiamidate, phosphorothiodiamidate, sulfamate, diethylenesulfoxide, amide, sulfonate, siloxane, sulfide, sulfone, formacetyl, alkenyl, methylenehydrazino, sulfonamide, triazole, oxalyl, carbamate, methyleneimino (MMI), and thioacetamide nucleic acid (TANA); and their derivatives. Various salts, mixed salts, deprotonated, protonated, tautomeric, and free acid forms are also included, as well as 3′→3′ and 2′→5′ linkages. An AON as disclosed herein may also comprise one or more linkage modifications according to the structure of formulas (I), (II), (III), IV), or (V).

In a preferred aspect, the AON as disclosed herein comprises an internucleoside linkage of the structure of formula (I), wherein X═O and R═CH₃, which linkage is generally referred to herein as a PNms linkage. In other preferred aspects, R equals one of the following structures (a), (b), (c), (d), (e), (f), (g), (h), or (i):

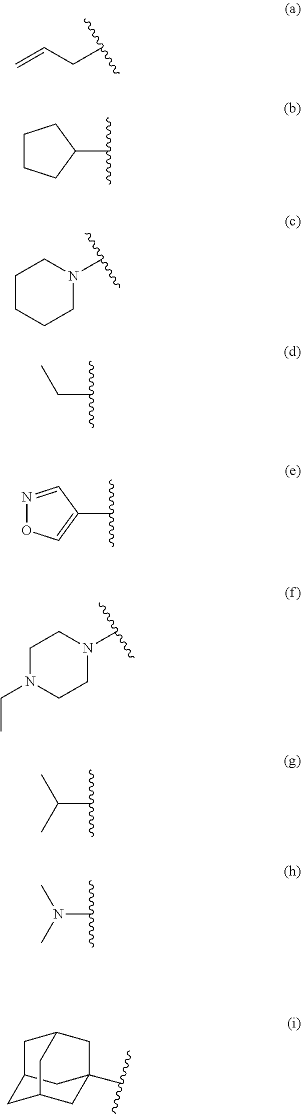

The one or more PN linkages as depicted in formula (I), present in an AON as disclosed herein, can be independently of each other of R_{P} or S_{P} chirality, or stereorandom.

The one or more PN linkages as depicted in formula (I), in an AON as disclosed herein, can be in tautomeric and/or pH-dependent (de) protonated form, including but not limited to the structures (A), (B), (C), (D), and (E):

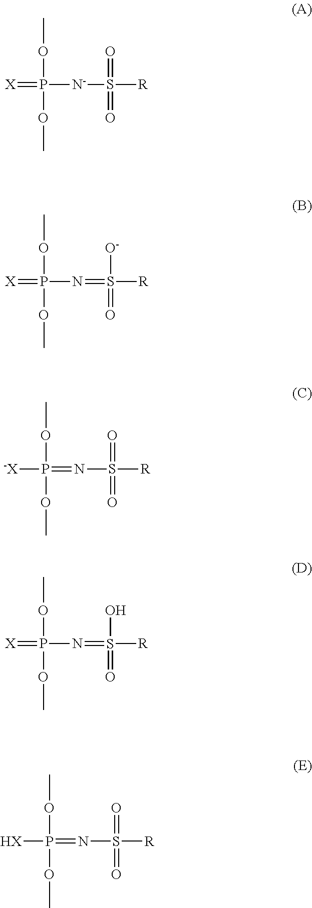

wherein X and R are as indicated above for formula (I).

Disclosed herein is also an AON that is able to mediate adenosine deamination by recruitment of a deaminating enzyme in a cell after the AON has formed a double-stranded complex with a region of a target RNA nucleic acid molecule in a cell, wherein the region comprises a target adenosine, wherein the deaminating enzyme can deaminate the target adenosine into an inosine, and wherein the AON comprises a moiety at one and/or both termini with a structure according to formula (II):

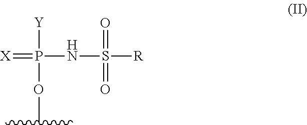

wherein: X═O or S;
Y═O′ or S⁻; and
R=an aryl, a substituted aryl, a heterocycle, a substituted heterocycle, an aromatic heterocycle, a substituted aromatic heterocycle, a C₁-C₆ alkoxy, a substituted C₁-C₆ alkoxy, a C₁-C₂₀ alkyl, a substituted C₁-C₂₀ alkyl, a C₁-C₆ alkenyl, a C₁-C₆ substituted alkenyl, a C₁-C₆ alkynyl, a substituted C₁-C₆ alkynyl, or a conjugate group. In a preferred embodiment, X═O and R=methyl. A preferred internucleoside linkage modification that is used in the AON as disclosed herein has the structure of formula (III):

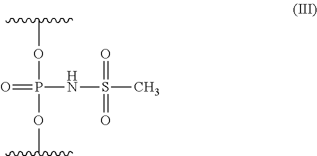 [SMILES: *OP(=O)(NS(C)(=O)=O)O*]

which is also referred to as a PNms linkage. PNms linkages and their application in oligonucleotides and as replacement of PS linkages has been described (Chelobanov B P et al. Russian J Bioorganic Chemistry. 2017. 43(6):664-668; DOI: 10.1134/S1068162017060024; Klabenkova K et al. Molecules. 2021. 26(17): 5420; Miroshnichenko S K et al. Proc Natl Acad Sci USA. 2019. 116 (4): 1229-1234), and for instance in oligonucleotides that may provide splice switching (Hammond S M et al. Nucleic Acid Ther. 31(3):190-200).

An AON as disclosed herein may comprise a substitution of one of the non-bridging oxygens in the phosphodiester linkage. This modification slightly destabilizes base pairing but adds significant resistance to nuclease degradation. A preferred nucleotide analogue or equivalent comprises PS, phosphonoacetate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate. Particularly preferred are internucleoside linkages that are modified to contain a PS. Particularly preferred are internucleoside linkages that are modified to contain a PNms. Particularly preferred are internucleoside linkages that are modified to contain a PNdmi. The regular internucleosidic linkages between the nucleotides may be altered by mono- or di-thioation of the phosphodiester bonds to yield PS esters or phosphorodithioate esters, respectively. Other modifications of the internucleosidic linkages are possible, including amidation and peptide linkers. The skilled person can determine for what target RNA nucleic acid molecule the AON comprises a certain linkage modification at each linkage position of the AON as disclosed herein to generate the most effective and most stable oligonucleotide compound.

Many of the non-naturally occurring modifications of the linkage, such as PS, are chiral. This means that there are Rp and Sp configurations, known to the person skilled in the art. In one embodiment, the chirality of the PS linkages is controlled, which means that each of the linkages is either in the Rp or in the Sp configuration, whichever is preferred. The choice of an Rp or Sp configuration at a specified linkage position may depend on the target sequence and the efficiency of binding and induction of causing RNA editing of the target adenosine. However, if such is not specifically desired, a composition may comprise AONs as active compounds with both Rp and Sp configurations at a certain specified linkage position. Mixtures of such AONs are also feasible, wherein certain positions preferably have either one of the configurations, while for other positions such does not matter. In one aspect, the AON as disclosed herein comprises one or more (chirally pure or chirally mixed) PS linkages. In one aspect, the AON as disclosed herein comprises one of more (chirally pure or chirally mixed) phosphoramidate (PN) linkages. In one aspect, the AON as disclosed herein comprises one or more (chirally pure or chirally mixed) PNms linkages. In one aspect, a PN linkage connects the terminal two nucleotides on each end of the AON. AONs as disclosed herein may also comprise linkage modifications at all positions that are not chirally controlled. The AON as disclosed herein may also comprise one or more naturally occurring internucleoside linkages. The choice and number of modified linkages may depend on the specific target, the sequence, the length, and the stability of the AON observed in a particular cell type of interest, which can be assessed by methods known to the person skilled in the art. In one aspect, at least one, at least two, at least three, or at least four internucleoside linkages between the 5′ and/or the 3′ terminal two, three, four, or five nucleosides respectively of the AON as disclosed herein are modified internucleoside linkages. In one aspect, the AON as disclosed herein comprises at least one MP internucleoside linkage according to the structure of formula (IV):

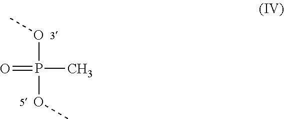

As was noted in the art, a preferred position for an MP linkage in an AON is linkage position-2, thereby connecting the nucleoside at position-1 with the nucleoside at position-2. In a preferred embodiment, this position, in an AON as disclosed herein, comprises a linkage modification according to the structure of formula (I), more preferably a linkage modification according to the structure of formula (III), instead of an MP linkage. WO2020/201406 discloses the use of MP linkage modifications at certain positions surrounding the orphan nucleotide in the first nucleic acid strand. Although the presence of MP linkages is compatible with RNA editing by human ADAR enzymes, introducing MP linkages during the manufacturing of oligonucleotides is challenging in view of additional manufacturing (purification) steps in the coupling and decoupling process. In one aspect, the AON does not comprise an MP linkage.

In one aspect, the AON as disclosed herein comprises at least one PNdmi linkage, preferably linking the most terminal two nucleosides at the 5′ and/or 3′ end of the AON. A PNdmi linkage that can be used in an AON as disclosed herein has the structure of formula (V):

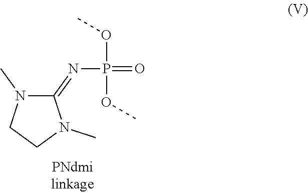

In one aspect, at either end or both termini of an AON as disclosed herein, inverted deoxyT or dideoxyT nucleotides are incorporated. Other internucleoside linkages that may be used in the AONs as disclosed herein are those that are disclosed in WO2023/278589.

In one aspect, the AON as disclosed herein comprises at least one phosphonoacetate and/or at least one phosphonoacetamide internucleoside linkage.

### Conjugate Chemistries

In one aspect, the AON as disclosed herein, or the sense strand to which it may be annealed before entering a target cell (in an HEON as disclosed herein), is bound to a hydrophobic moiety, such as palmityl or an analog thereof, cholesterol or analog thereof, or tocopherol or analog thereof. It is preferably bound to the 5′ terminus. In case a hydrophobic moiety is bound to the 5′ terminus as well as to the 3′ terminus, such hydrophobic moieties may the same or different. The hydrophobic moiety bound to the oligonucleotide may be bound directly, or indirectly mediated by another substance. When the hydrophobic moiety is bound directly, it is sufficient if the moiety is bound via a covalent bond, an ionic bond, a hydrogen bond, or the like. When the hydrophobic moiety is bound indirectly, it may be bound via a linking group (a linker). The linker may be a cleavable or an uncleavable linker. A cleavable linker refers to a linker that can be cleaved under physiological conditions, for example, in a cell or an animal body (e.g., a human body). A cleavable linker is selectively cleaved by an endogenous enzyme such as a nuclease, or by physiological circumstances specific to parts of the body or cell, such as pH or reducing environment (such as glutathione concentrations). Examples of a cleavable linker comprise, but is not limited to, an amide, an ester, one or both esters of a phosphodiester, a phosphoester, a carbamate, and a disulfide bond, as well as a natural DNA linker. Cleavable linkers also include self-immolative linkers. An uncleavable linker refers to a linker that is not cleaved under physiological conditions, or very slowly compared to a cleavable linker, for example, in a PS linkage, modified or unmodified deoxyribonucleosides linked by a PS linkage, a spacer connected through a PS bond and a linker consisting of modified or unmodified ribonucleosides. There is no restriction on the chain length, when a linker is a nucleic acid such as DNA, or an oligonucleotide. However, it may be usually from 2 to 20 bases in length, from 3 to 10 bases in length, or from 4 to 6 bases in length. There is no restriction on the length or composition of a spacer that is connects the ligand and the oligonucleotide, and may include for example ethylene glycol, triethylene glycol (TEG), HEG, alkyl chains, propyl, 6-aminohexyl, or dodecyl. In one embodiment, a GalNAc moiety is bound to the AON as disclosed herein via a TEG linker. One or more other types of molecules may be bound to the AON through one or more linkers, including peptides, sugars, vitamins, polymers, aptamers, (fragments of) antibodies, small molecules, and the like.

### General

In addition to the specific preferred chemical modifications at certain positions in compounds as disclosed herein, AONs as disclosed herein may comprise one or more (additional) modifications to the nucleobase, scaffold and/or backbone linkage, which may or may not be present in the same monomer, for instance at the 3′ and/or 5′ position. In one aspect, the AON as disclosed herein comprises at least one internucleoside linkage according to the structure of formula (I), and/or the AON further comprises at least one nucleotide with a sugar moiety that comprises a 2′-OMe modification, and/or the AON comprises at least one nucleotide with a sugar moiety that comprises a 2′-MOE modification, and/or the AON comprises at least one nucleotide with a sugar moiety that comprises a 2′-F modification, and/or the AON comprises an orphan nucleotide that carries a 2′-H in the sugar moiety and is therefore referred to as a DNA nucleotide, even though additional modifications may exist in its base and/or linkage to its neighbouring nucleosides. In one aspect, the orphan nucleotide carries a 2′-F in the sugar moiety. In one aspect, the orphan nucleotide carries a diF substitution in the sugar moiety. In one aspect, the orphan nucleotide carries a 2′-F and a 2′-C-methyl in the sugar moiety. In one aspect, the orphan nucleotide comprises a 2′-F in the arabinose configuration (FANA) in the sugar moiety.

In one aspect, the AON is an antisense oligonucleotide that can form a double stranded nucleic acid complex with a target RNA molecule, wherein the double stranded nucleic acid complex can recruit an adenosine deaminating enzyme for deamination of a target adenosine in the target RNA molecule, wherein the nucleotide in the AON that is opposite the target adenosine is the orphan nucleotide, and wherein the orphan nucleotide has the structure of formula (VI):

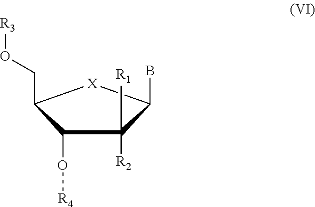

wherein: X is O, NH, OCH₂, CH₂, Se, or S; B is a nitrogenous base selected from the group consisting of: cytosine, uracil, isouracil, N3-glycosylated uracil, pseudoisocytosine, 8-oxo-adenine, and 6-amino-5-nitro-3-yl-2 (1H)-pyridone; R₁ and R₂ are both selected, independently, from H, OH, F or CH₃; R₃ is the part of the AON that is 5′ of the orphan nucleotide, consisting of 7 to 30 nucleotides; and R₄ is the part of the AON that is 3′ of the orphan nucleotide, consisting of 4 to 25 nucleotides. The nucleotide 3′ and/or 5′ from the orphan nucleotide may be DNA, more preferably the nucleotide at the 3′ (position-1).

Other chemical modifications of the AON as disclosed herein include the substitution of one or more than one of any of the hydrogen atoms with deuterium or tritium, examples of which can be found in e.g., WO2014/022566 or WO2015/011694. Again, in all cases, the modifications should be compatible with editing such that the AON fulfils its role as an oligonucleotide that can, after binding to its target sequence, recruit an adenosine deaminase enzyme because of the double-stranded nucleic acid entity that arises. In all aspects of the disclosure, the enzyme with adenosine deaminase activity is preferably ADAR1, ADAR2, or ADAT.

AONs as disclosed herein preferably do not include a 5′-terminal 06-benzylguanosine or a 5′-terminal amino modification and preferably are not covalently linked to a SNAP-tag domain (an engineered O6-alkylguanosine-DNA-alkyl transferase). An AON as disclosed herein preferably does not comprise a boxB RNA hairpin sequence. In one aspect, an AON as disclosed herein comprises 0, 1, 2 or 3 wobble base pairs with the target sequence, and/or 0, 1, 2, 3, 4, 5, 6, 7, or 8 mismatching base pairs with the target RNA sequence. No mismatch exists when the orphan nucleotide is uridine, which may be defined differently when the orphan nucleotide is a uridine analog or derivative. One alternative for uridine is positioning an iso-uridine opposite the target adenosine, which likely does not pair like G pairs with U. Preferably, the target adenosine in the target sequence forms a mismatch base pair with the nucleoside in the AON that is directly opposite the target adenosine.

As outlined above, an AON as disclosed herein makes use of specific nucleotide modifications at predefined spots to ensure stability as well as proper ADAR binding and activity. These changes may vary and may include modifications in the backbone of the AON, in the sugar moiety of the nucleotides as well as in the nucleobases or the phosphodiester linkages, as outlined in detail herein. They may also be variably distributed throughout the sequence of the AON. Specific modifications may be needed to support interactions of different amino acid residues within the RNA-binding domains of ADAR enzymes, as well as those in the deaminase domain. For example, PS linkages between nucleotides or 2′-OMe or 2′-MOE modifications may be tolerated in some parts of the AON, while in other parts they should be avoided so as not to disrupt crucial interactions of the enzyme with the phosphate and 2′—OH groups. Specific nucleotide modifications may also be necessary to enhance the editing activity on substrate RNAs where the target sequence is not optimal for ADAR editing. Previous work has established that certain sequence contexts are more amenable to editing. For example, a target sequence 5′-UAG-3′ (with the target A in the middle) contains the most preferred nearest-neighbor nucleotides for ADAR2, whereas a 5′-CAA-3′ target sequence is disfavored (Schneider et al. 2014. Nucleic Acids Res 42 (10): e87). The structural analysis of ADAR2 deaminase domain hints at the possibility of enhancing editing by careful selection of the nucleotides that are opposite to the target trinucleotide. For example, the 5′-CAA-3′ target sequence, paired to a 3′-GCU-5′ sequence on the opposing strand (with the A-C mismatch formed in the middle), is disfavored because the guanosine base sterically clashes with an amino acid side chain of ADAR2. The guanosine opposite the C in such circumstances is preferably replaced by an inosine (hence, at the −1 position within the AON), more preferably a deoxyinosine.

The AON as disclosed herein, in contrast to what has been described for siRNA, or gapmers and their relation towards RNase breakdown and the use of such gapmers in double-stranded complexes (see for instance EP 3954395 A1), does not comprise a stretch of DNA nucleotides which would make a target sequence (or a sense nucleic acid strand) a target for RNase-mediated breakdown. It is not desired that the target transcript molecule is degraded through the binding of the AON to the transcript molecule. In one embodiment, the AON does not comprise four or more consecutive DNA nucleotides anywhere within its sequence. In an embodiment, the AON is composed of as much (chemically) modified nucleotides as possible to enhance the resistance towards RNase-mediated breakdown, while at the same time being as efficient as possible in producing an RNA editing effect. This means that the orphan nucleotide and several other nucleotides within the AON may be DNA, but also that there is no stretch of four or more consecutive DNA nucleotides within the AON. Hence, the AON as disclosed herein is not a gapmer. A gapmer reduces the expression of a target transcript but does not produce RNA editing of a specified adenosine within the target transcript. A gapmer is in principle a single-stranded nucleic acid consisting of a central region (DNA gap region with at least four consecutive deoxyribonucleotides) and wing regions positioned directly at the 5′ end (5′ wing region) and the 3′ end (3′ wing region) thereof. In contrast, the AON as disclosed herein may be any oligonucleotide that produces an RNA editing effect in which a target adenosine in a target RNA molecule is deaminated to an inosine, and accordingly is resistant to RNase-mediated breakdown as much as possible to yield this effect and to allow the mRNA transcript being translated into a protein.

The AONs as disclosed herein may also be administered in the context of aids that will increase the entry of the AON into the target cell and/or its endosomal escape as soon as it is in the cell. Moieties that can be applied for such applications are for example a set of chemical compounds (generally purified from nature) referred to as “saponins” or “triterpene glycosides”. A preferred saponin that can be used in the methods as disclosed herein is AG1856, disclosed in WO2021/122998 and further described for use with RNA editing producing oligonucleotides in PCT/EP2024/051278 (unpublished).

Disclosed herein is also a pharmaceutical composition comprising the AON as disclosed herein, and further comprising a pharmaceutically acceptable carrier, solvent, diluent, and/or other additive (such as a saponin or triterpene glycoside like AG1856 (as discussed above), which in fact may also be administered separately from the AON) and may be dissolved in a pharmaceutically acceptable organic solvent, or the like. Dosage forms in which the AON or the pharmaceutical composition are administered may depend on the disorder to be treated and the tissue that needs to be targeted and can be selected according to common procedures in the art. The pharmaceutical compositions may be administered by a single-dose administration or by multiple dose administration. It may be administered daily or at appropriate time intervals, which may be determined using common general knowledge in the field and may be adjusted based on the disorder and the efficacy of the active ingredient.

Although in a preferred embodiment, the AON as disclosed herein is a single-stranded oligonucleotide comprising an orphan nucleotide opposite the target adenosine, wherein the orphan nucleotide is chemically modified as disclosed herein, and wherein the remainder of the oligonucleotide is chemically modified to prevent it from nuclease breakdown also as disclosed herein, in another embodiment, disclosed is any kind of oligonucleotide or heteroduplex oligonucleotide complex, that may or may not be bound to hairpin structures (internally or at the terminal end(s)), that may be bound to ADAR or catalytic domains thereof, or wherein the oligonucleotide is in a circular format. In a preferred aspect, the AON as disclosed herein is a ‘naked’ oligonucleotide, comprising a variety of chemical modifications in the ribose sugar and/or the base of one or more of the nucleotides within the sequence, that preferably comprises at least one linkage according to the structure of formula (I) as disclosed herein, that can hybridize to the target transcript or a part thereof that includes the target adenosine, and can recruit endogenous (naturally present) ADAR in the target cell for the deamination of the target adenosine. In another aspect, the AON as disclosed herein, that is delivered in a ‘naked’ form, does not comprise a stem-loop structure for recruitment of the deaminating enzyme, which allows for a shorter AON and improved cellular delivery and trafficking.

It is known in the art that RNA editing entities (such as human ADAR enzymes) edit dsRNA structures with varying specificity, depending on several factors. One important factor is the degree of complementarity of the two strands making up the dsRNA sequence. Perfect complementarity of the two strands usually causes the catalytic domain of human ADAR to deaminate adenosines in a non-discriminative manner, reacting with any adenosine it encounters. The specificity of hADAR1 and 2 can be increased by introducing chemical modifications and/or ensuring several mismatches in the dsRNA, which presumably helps to position the dsRNA binding domains in a way that has not been clearly defined yet. Additionally, the deamination reaction itself can be enhanced by providing an oligonucleotide that comprises a mismatch opposite the adenosine to be edited. Following the instructions in the present application, those of skill in the art will be capable of designing the complementary portion of the oligonucleotide according to their needs.

It will be understood by a person having ordinary skill in the art that the extent to which the editing enzymes inside the cell are redirected to other target sites may be regulated by varying the affinity of the first nucleic acid strand for the recognition domain of the editing enzyme. The exact modification may be determined through some trial and error and/or through computational methods based on structural interactions between the AON and the recognition domain of the editing enzyme. In addition, or alternatively, the degree of recruiting and redirecting the editing enzyme resident in the cell may be regulated by the dosing and the dosing regimen of the AON. This is something to be determined by the experimenter (in vitro) or the clinician, usually in phase I and/or II clinical trials.

Disclosed herein is the site-specific editing of target adenosines in RNA sequences in eukaryotic, preferably metazoan, more preferably mammalian, more preferably human cells, more preferably human liver cells, and most preferably human hepatocytes. The target cell can be located in vitro, ex vivo or in vivo. One advantage of the AON as disclosed herein is that it can be used with cells in situ in a living organism, but it can also be used with cells in culture. In some embodiments cells are treated ex vivo and are then introduced into a living organism (e.g., re-introduced into an organism from whom they were originally derived). The AON as disclosed herein can also be used to edit target RNA sequences in cells from a transplant or within a so-called organoid, e.g., a liver tissue organoid. Organoids can be thought of as three-dimensional in vitro-derived tissues but are driven using specific conditions to generate individual, isolated tissues. In a therapeutic setting they are useful because they can be derived in vitro from a patient's cells, and the organoids can then be re-introduced to the patient as autologous material which is less likely to be rejected than a normal transplant.

Without wishing to be bound by theory, the RNA editing through human ADAR2 for example is thought to take place on primary transcripts in the nucleus, during transcription or splicing, or in the cytoplasm, where e.g., mature mRNA, miRNA or ncRNA can be edited. Generally spoken, RNA editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. These and other forms of RNA and protein “engineering”, whether to prevent, delay or treat disease or for any other purpose, in medicine or biotechnology, as diagnostic, prophylactic, therapeutic, research tool or otherwise, are encompassed by the present disclosure.

The amount of AON to be administered, the dosage and the dosing regimen can vary from cell type to cell type, the disease to be treated, the target population, the mode of administration (e.g., systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change, or a change in (the level of, or activity of) a specified biomarker (such as plasma levels of bile acids for example). It is possible that higher doses of AONs could compete for binding to an ADAR enzyme within a cell, thereby depleting the amount of the enzyme, which is free to take part in RNA editing, but routine dosing trials will reveal any such effects for a given AON and a given target.

One suitable trial technique involves delivering the AON to cell lines, or a test organism and then taking biopsy samples at various time points thereafter. The sequence of the target RNA can be assessed in the biopsy sample and the proportion of cells having the modification can easily be followed. As mentioned above, plasma level concentrations of bile acids in a sample from a treated subject is a proper biomarker for assessing the function of certain proteins in the subject, before and after treatment, or with or without treating the subject with an AON as disclosed herein. After this trial has been performed once then the knowledge can be retained, and future delivery can be performed without needing to take biopsy samples. A method as disclosed herein can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively, as indicated above, the change may be assessed on the function of the protein before, during, and/or after treatment or assessing any other potential marker, which measurements are preferably performed in vitro on samples obtained from the treated subject.

After RNA editing has occurred in a cell, the modified RNA can become diluted over time, for example due to cell division, limited half-life of the edited RNAs, etc. Thus, in practical therapeutic terms a method as disclosed herein may involve repeated delivery of an AON until enough target RNAs have been modified to provide a tangible benefit to the patient and/or to maintain the benefits over time.

AONs as disclosed herein are particularly suitable for therapeutic use, and so disclosed is also a pharmaceutical composition comprising an AON as disclosed herein and a pharmaceutically acceptable carrier, solvent, or diluent. In some embodiments the pharmaceutically acceptable carrier can simply be a saline solution. This can usefully be isotonic or hypotonic, particularly for pulmonary delivery. The AON as disclosed herein is suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g., through nebulization), intranasally, orally, by injection or infusion, intravenously, subcutaneously, intradermally, intramuscularly, intra-tracheally, intra-peritoneally, intrarectally, intrathecally, intra-cisterna magna, parenterally, and the like. Administration may be in solid form, in the form of a powder, a pill, a gel, a solution, a slow-release formulation, or in any other form compatible with pharmaceutical use in humans.

In one embodiment, depending on the ultimate deamination effect of A to I conversion, the identification step of whether the editing has taken place, comprises the following steps: sequencing the target RNA; assessing the presence or absence of a non-, or less-functional protein; assessing whether splicing of the pre-mRNA was altered by the deamination; or using a functional read-out, because the target RNA after the deamination should encode a protein with a lower or absent functionality, or on the other hand, an increased or regained functionality. The identification of the deamination into inosine may be a functional read-out using a suitable biomarker. A functional assessment will generally be according to methods known to the skilled person. A suitable manner to identify the presence of an inosine after deamination of the target adenosine is of course dPCR or even sequencing, using methods that are well-known to the person skilled in the art. However, the person skilled in the art of liver disease will preferably apply tests to monitor certain biomarkers related to liver function(s).

In one embodiment, a method as disclosed herein comprises the steps of administering to the subject an AON or pharmaceutical composition as disclosed herein, allowing the formation of a double stranded nucleic acid complex of the AON with its specific complementary target nucleic acid molecule in a cell in the subject; allowing the engagement of an endogenous present adenosine deaminating enzyme, such as ADAR 1 or ADAR2; and allowing the enzyme to deaminate the target adenosine in the target nucleic target molecule to an inosine, thereby alleviating, treating, ameliorating, or slowing down progression of the disease.

RNA editing molecules present in the cell will usually be proteinaceous in nature, such as the ADAR enzymes found in metazoans, including mammals. The ones of most interest are the human ADARs, hADAR1 and hADAR2, including any isoforms thereof. RNA editing enzymes known in the art, for which oligonucleotide constructs as disclosed herein may conveniently be designed, include the adenosine deaminases acting on RNA (ADARs), such as hADAR1 and hADAR2 in humans or human cells and cytidine deaminases. It is known that hADAR1 exists in two isoforms; a long 150 kDa interferon inducible version and a shorter, 110 kDa version, that is produced through alternative splicing from a common pre-mRNA. Consequently, the level of the 150 kDa isoform available in the cell may be influenced by interferon, particularly interferon-gamma (IFN-γ). hADAR1 is also inducible by TNF-α. This provides an opportunity to develop combination therapy, whereby IFN-γ or TNF-α and AONs as disclosed herein are administered to a patient either as a combination product, or as separate products, either simultaneously or subsequently, in any order. Certain disease conditions may already coincide with increased IFN-γ or TNF-α levels in certain tissues of a patient, creating further opportunities to make editing more specific for diseased tissues. It will be understood by a person having ordinary skill in the art that the extent to which the editing entities inside the cell are redirected to other target sites may be regulated by varying the affinity of the first nucleic acid strand for the recognition domain of the editing molecule.

An AON as disclosed herein can utilise endogenous cellular pathways and naturally available ADAR enzymes to specifically edit a target adenosine in the target RNA sequence. An AON as disclosed herein is capable of recruiting ADAR and complex with it and then facilitates the deamination of a (single) specific target adenosine nucleotide in a target RNA sequence to which it is bound. Ideally, only one adenosine is deaminated. An AON as disclosed herein, when complexed to ADAR, preferably brings about the deamination of a single target adenosine.

An AON as disclosed herein, especially when it is in a naked form, is normally longer than 10 nucleotides, preferably more than 11, 12, 13, 14, 15, 16, still more preferably more than 17 nucleotides. In one aspect the AON as disclosed herein is longer than 20 nucleotides. The AON as disclosed herein is preferably shorter than 100 nucleotides, still more preferably shorter than 60 nucleotides, still more preferably shorter than 50 nucleotides. In a preferred aspect, the AON as disclosed herein comprises 18 to 70 nucleotides, more preferably comprises 18 to 60 nucleotides, and even more preferably comprises 18 to 50 nucleotides. Hence, in a particularly preferred aspect, the AON as disclosed herein comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides. In one embodiment, the AON is 27, 28, 29, or 30 nucleotides in length.

### Examples

### Example 1. RNA Editing of the SLC10A1 Transcript Using a Variety of AONs

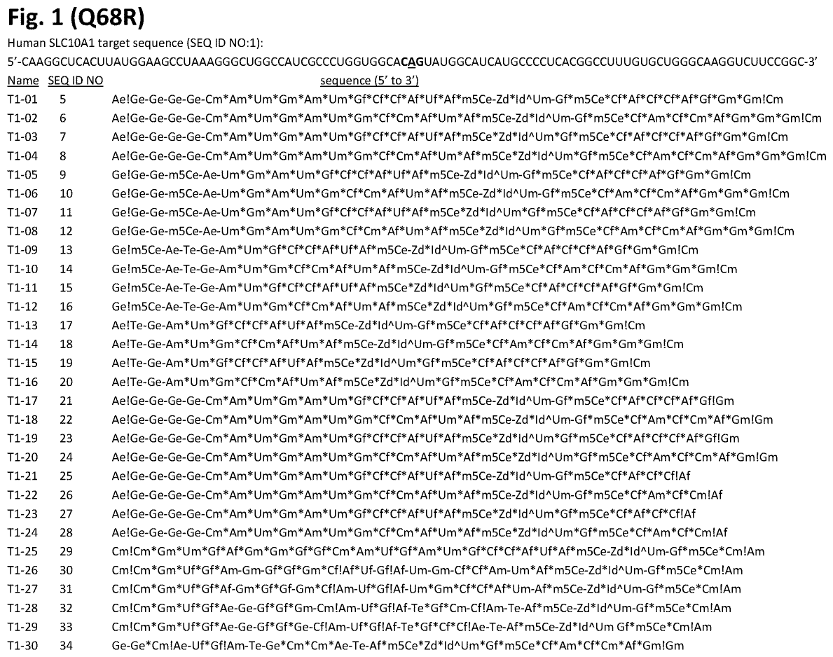

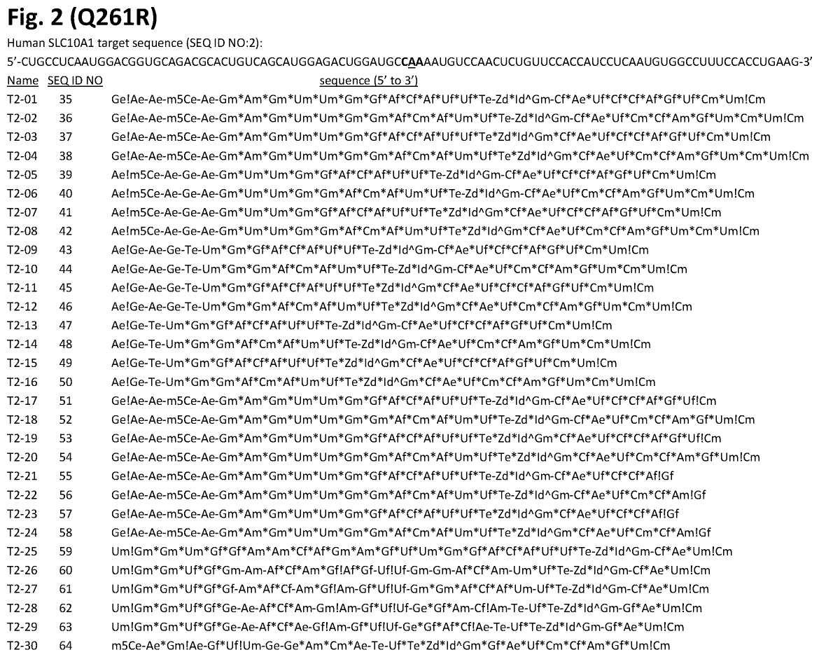

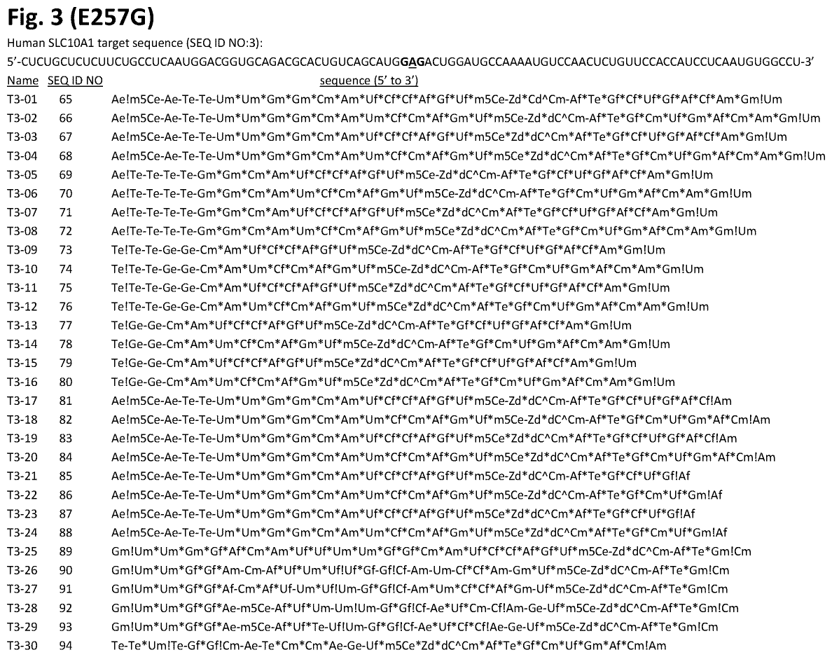

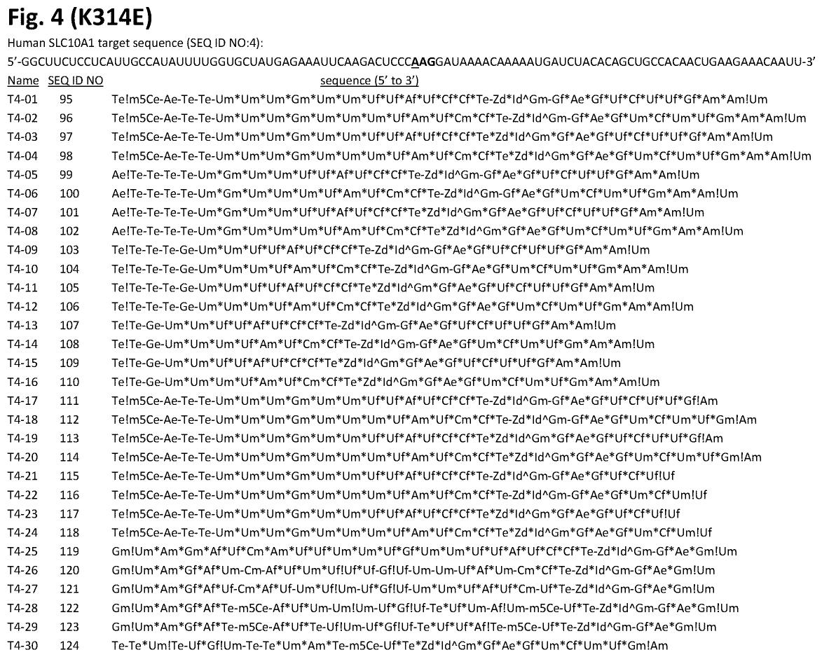

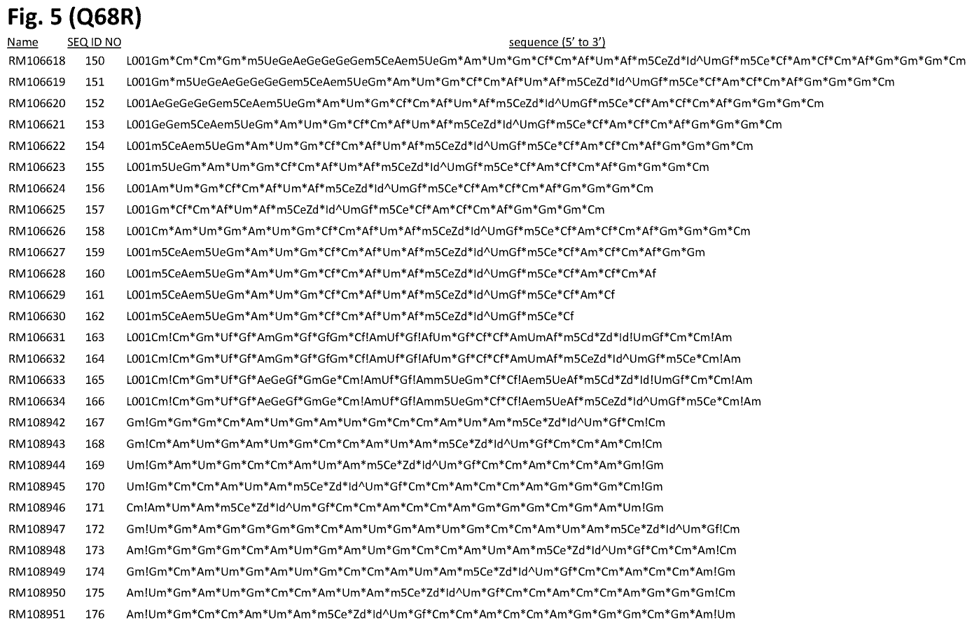

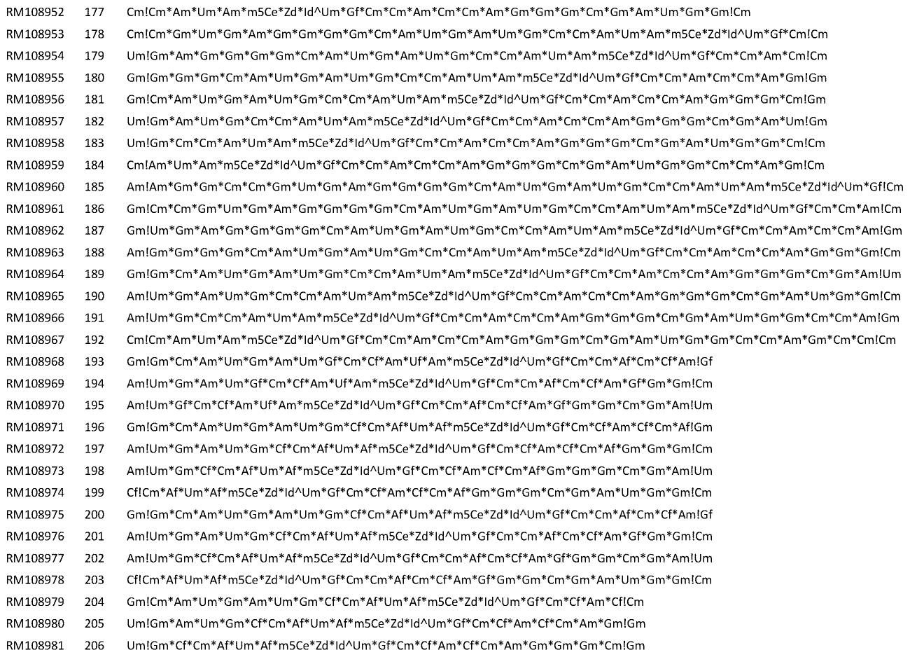

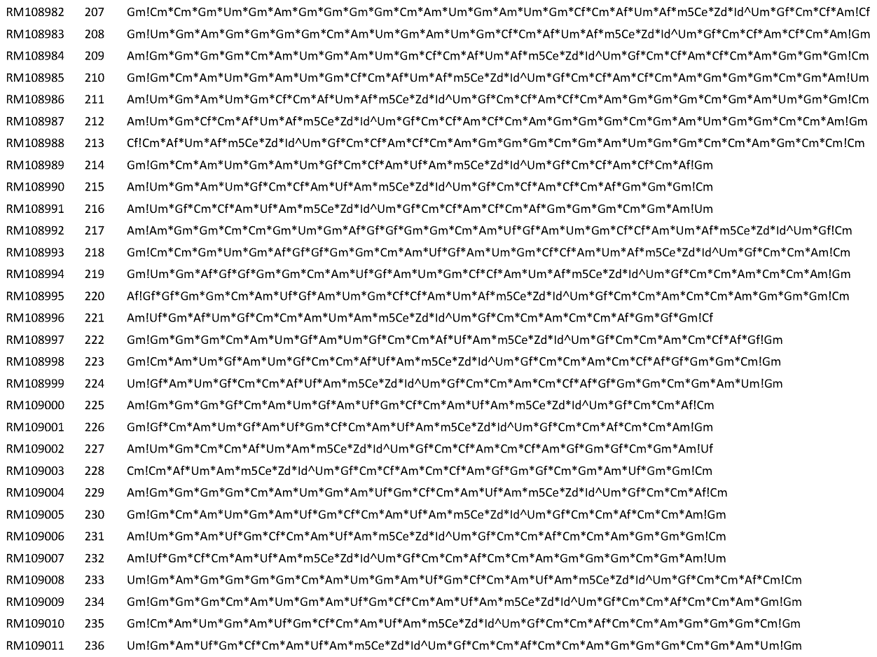

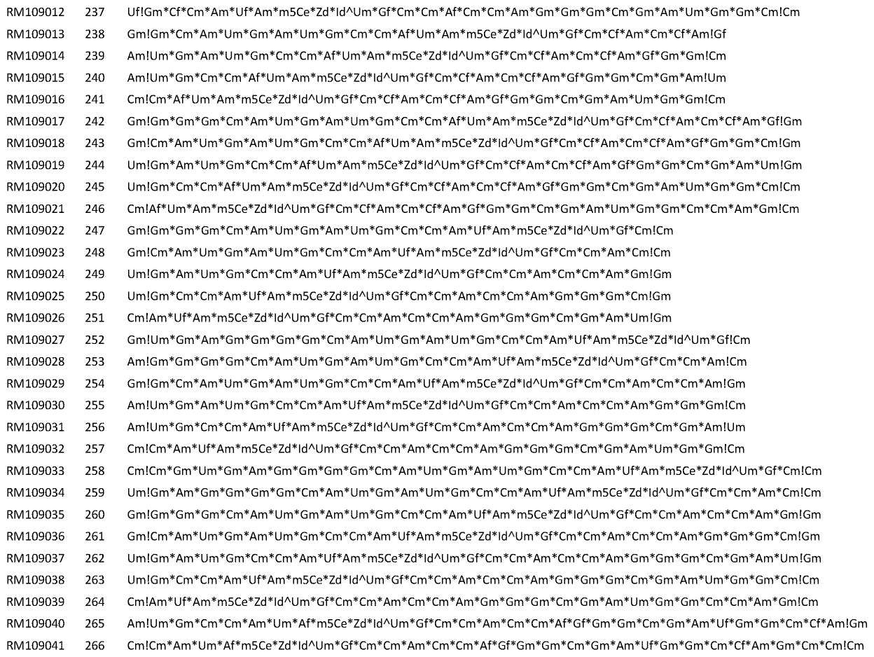

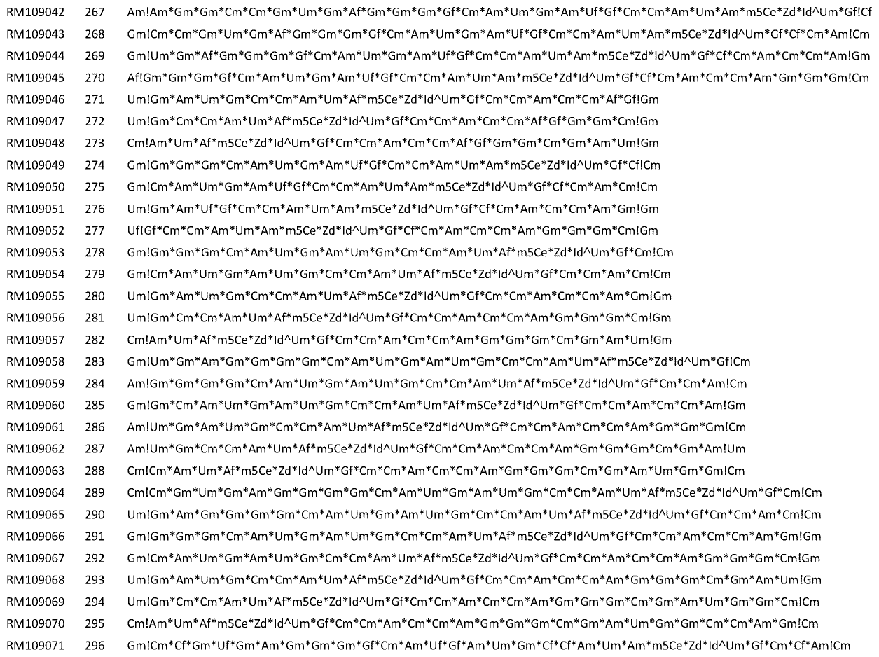

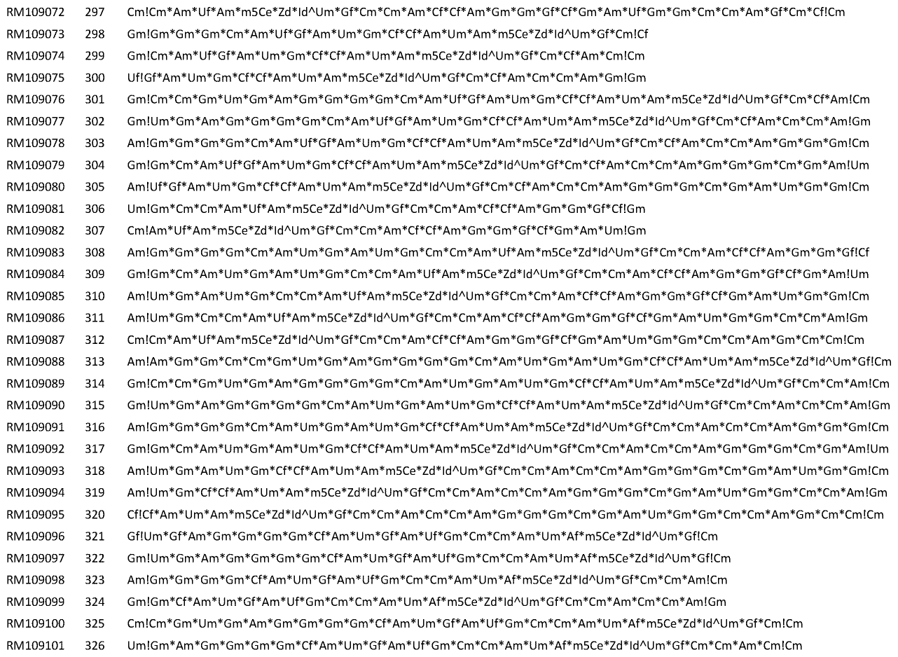

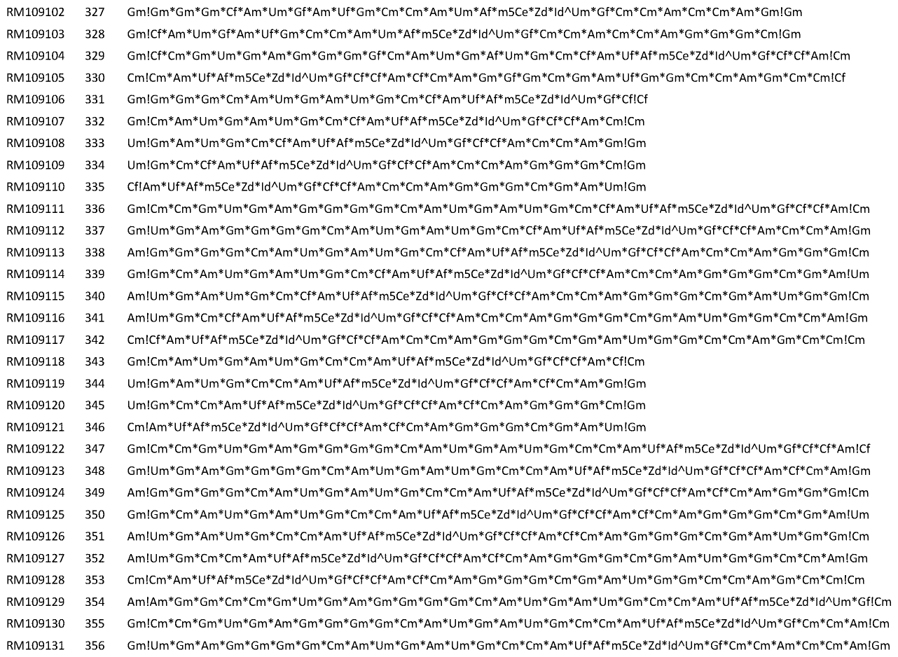

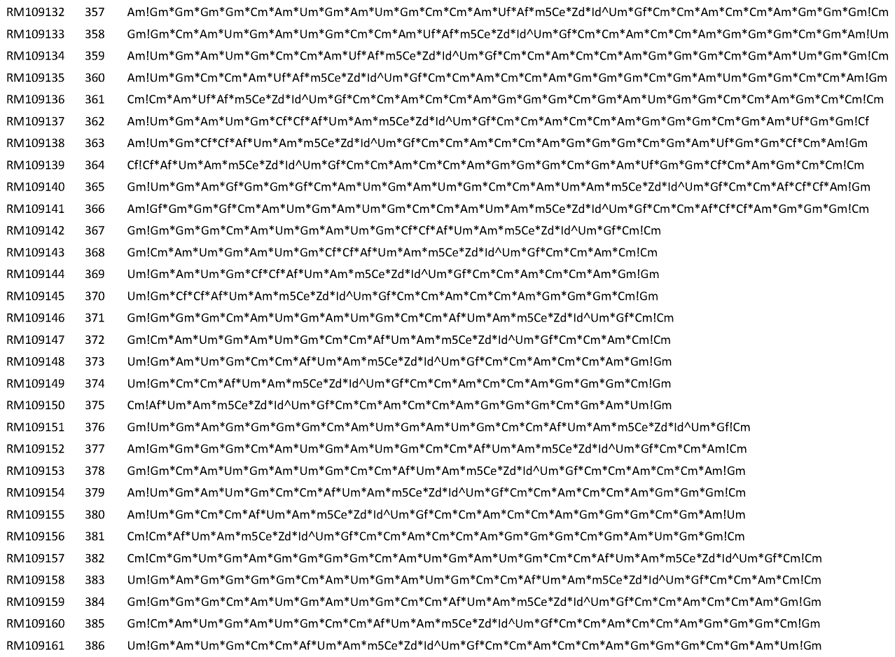

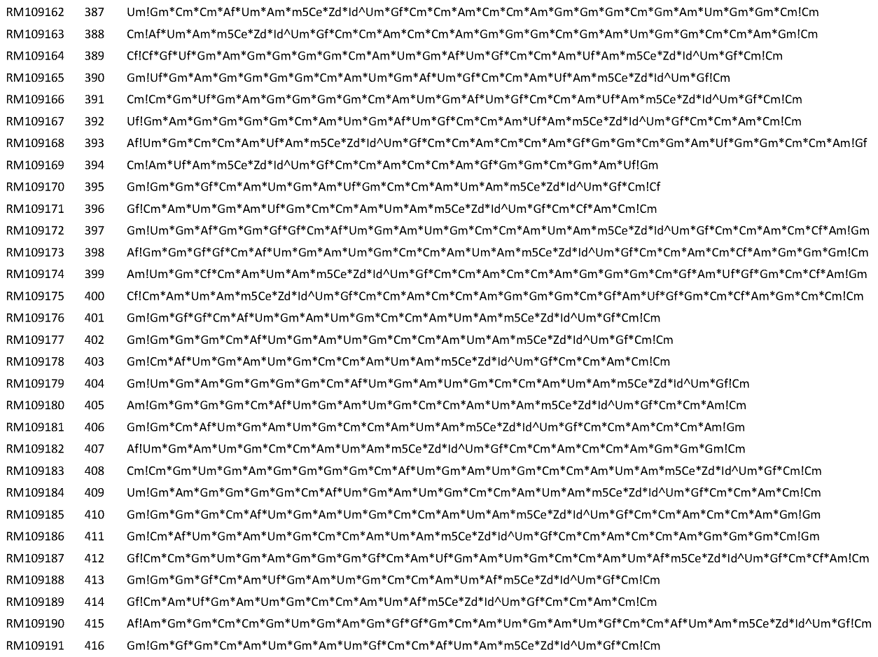

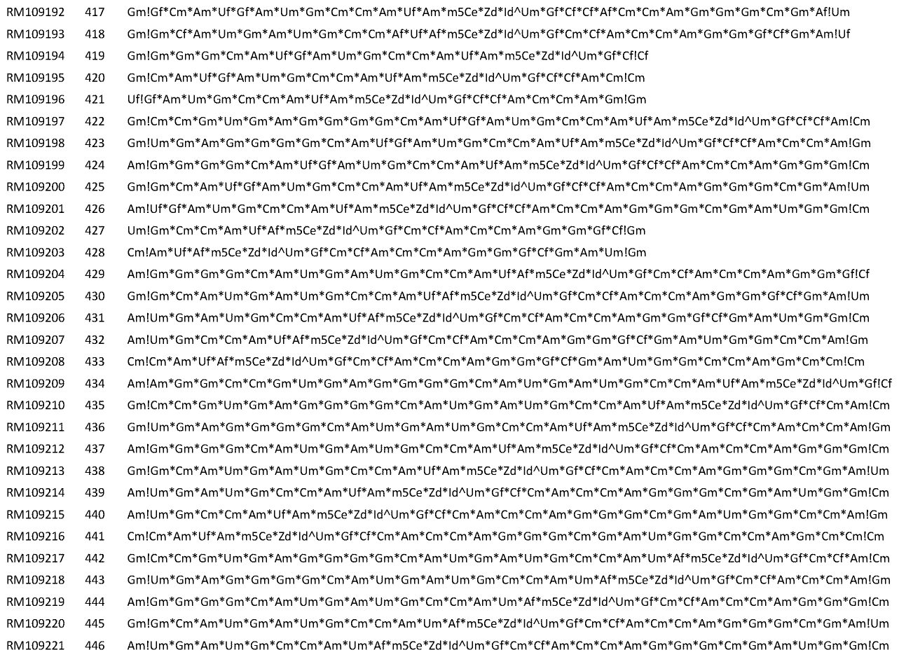

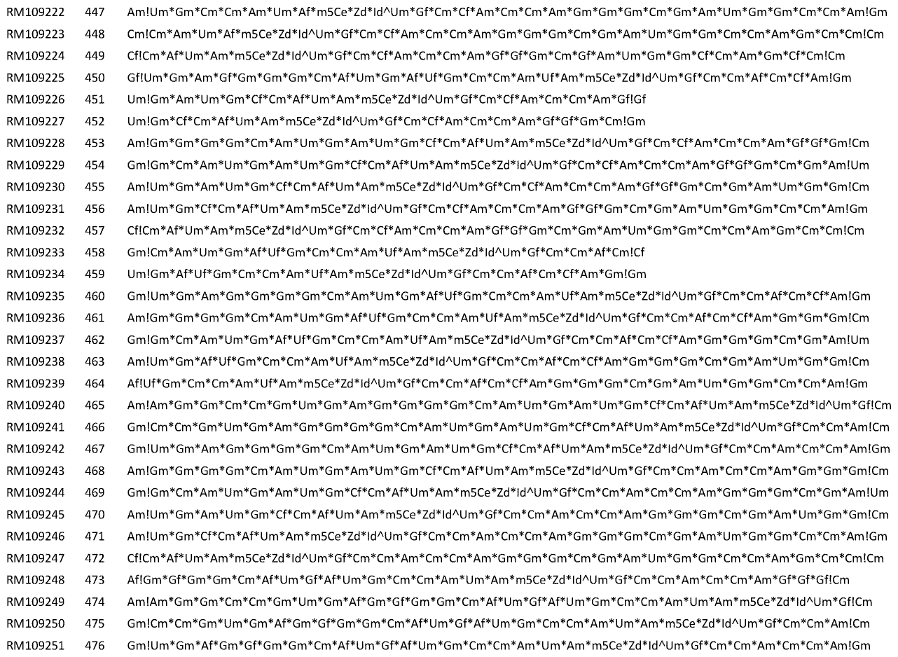

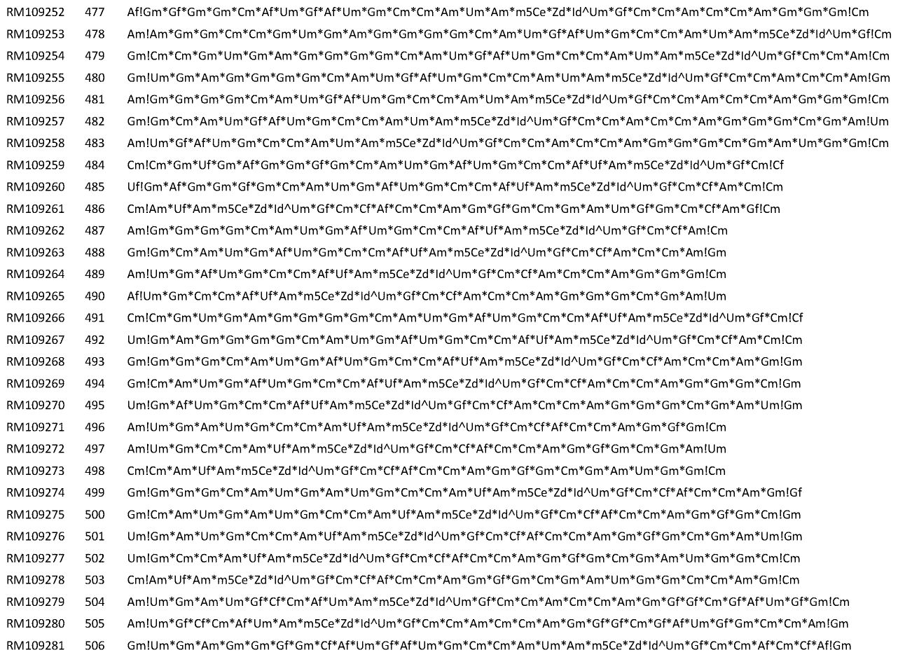

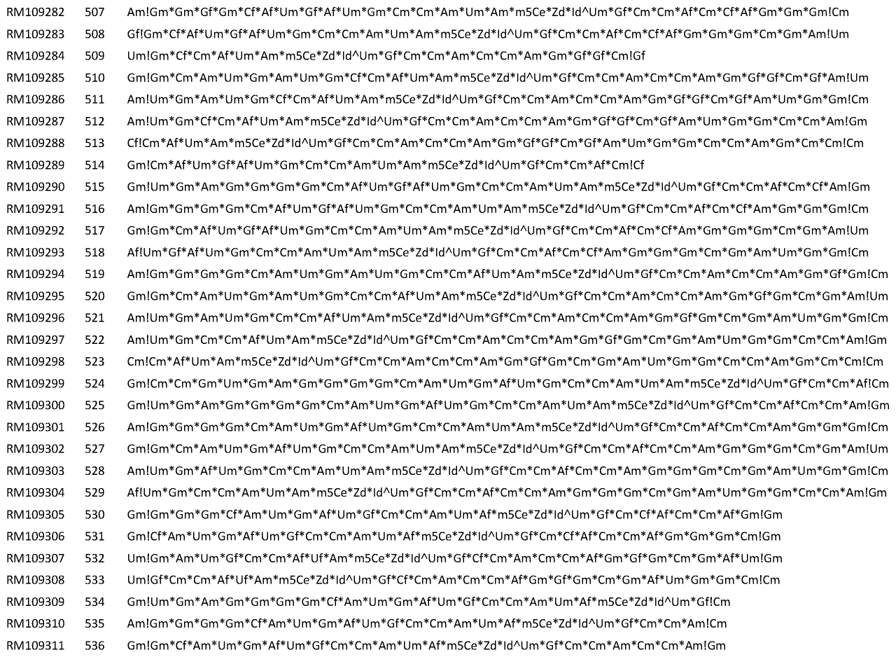

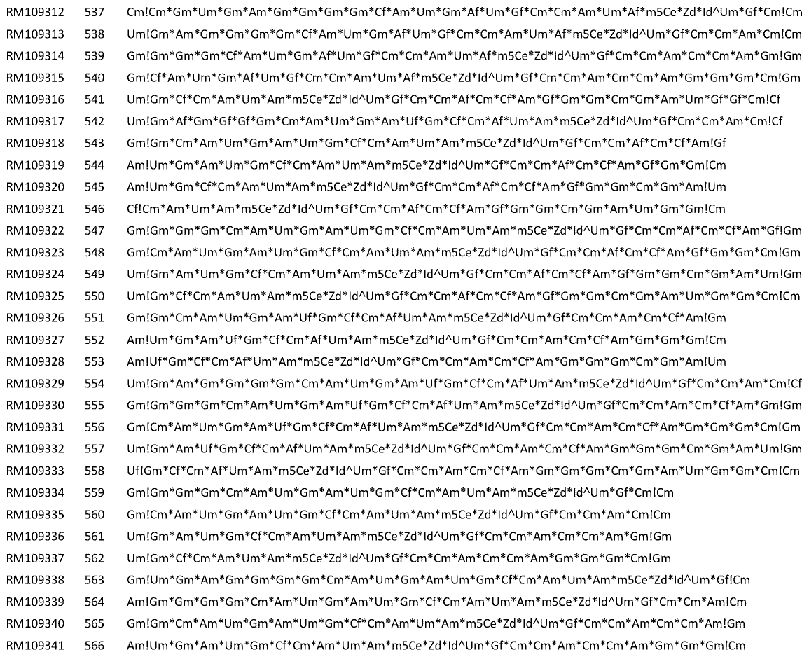

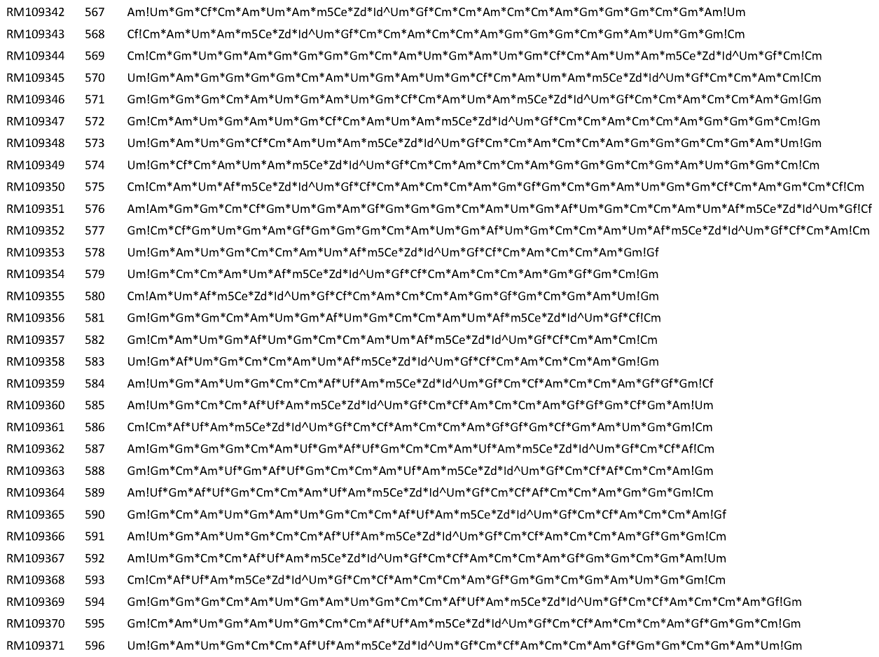

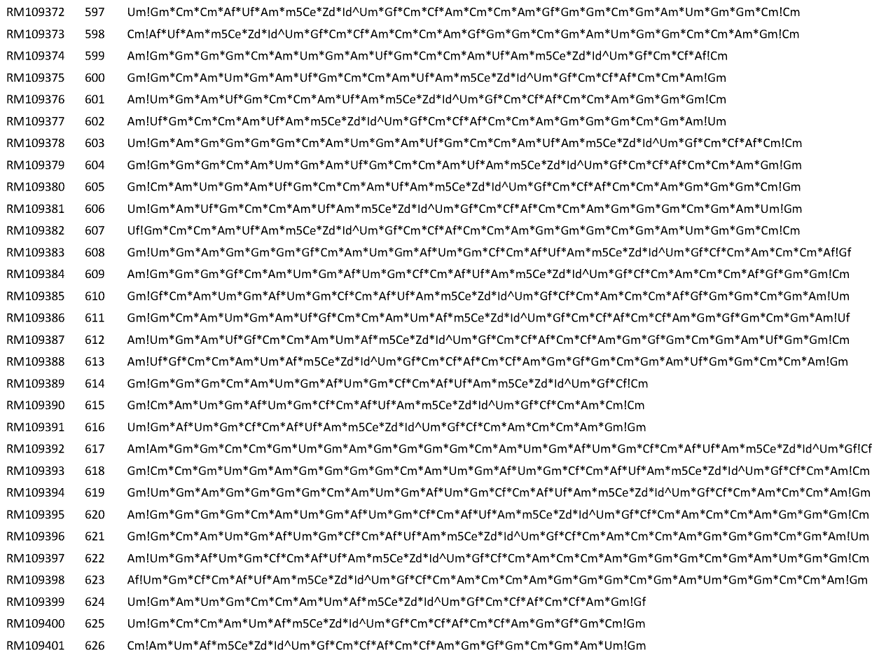

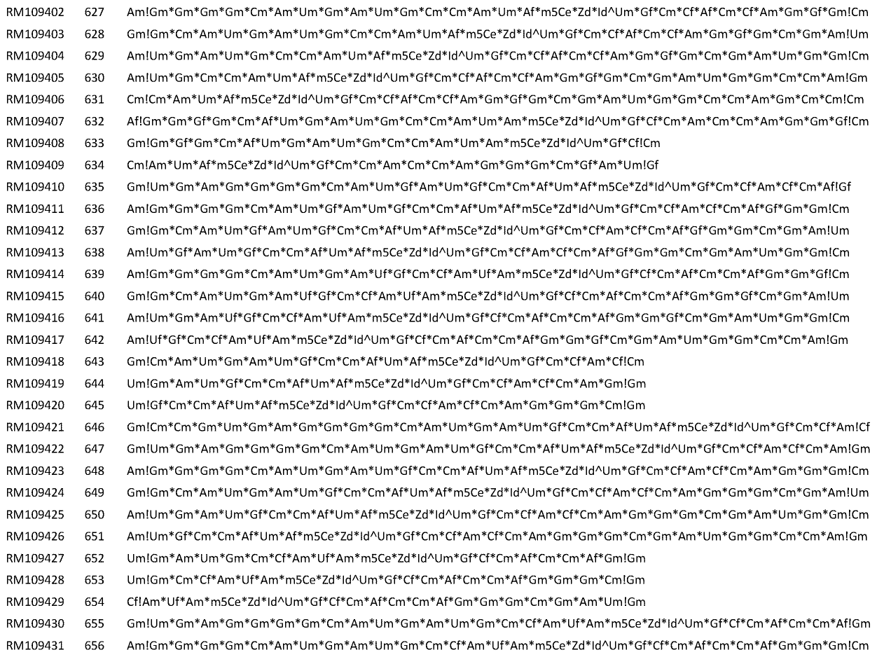

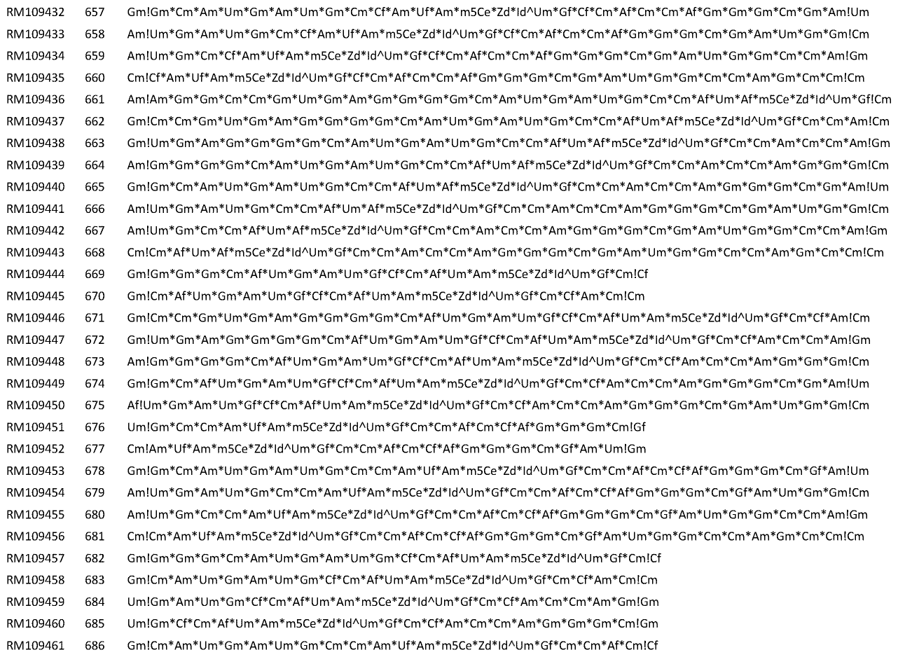

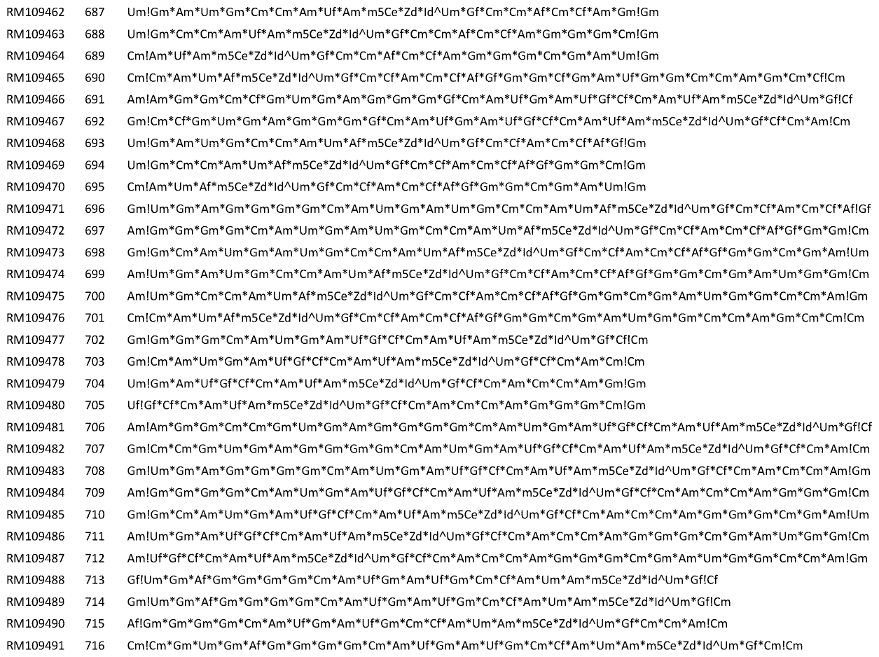

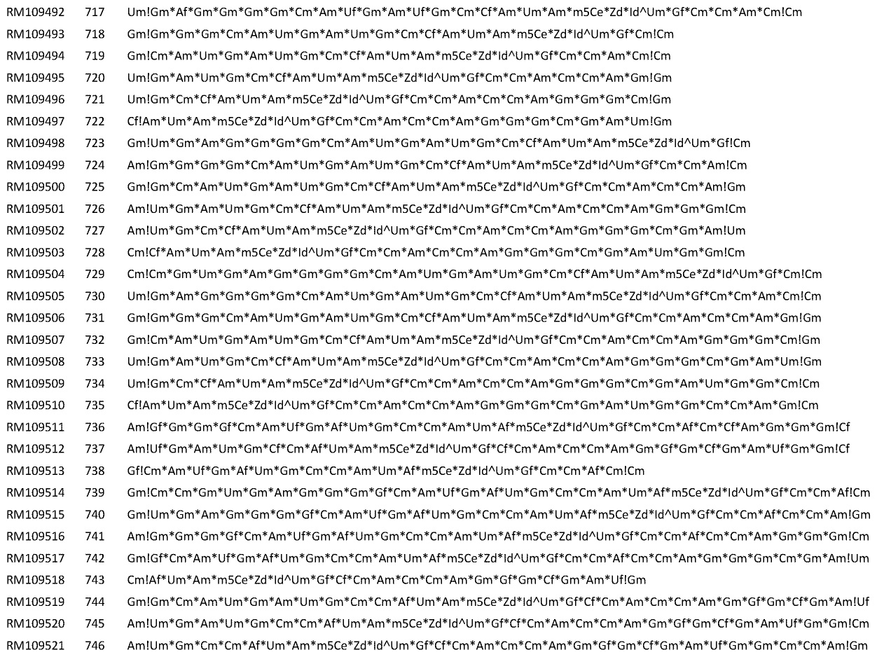

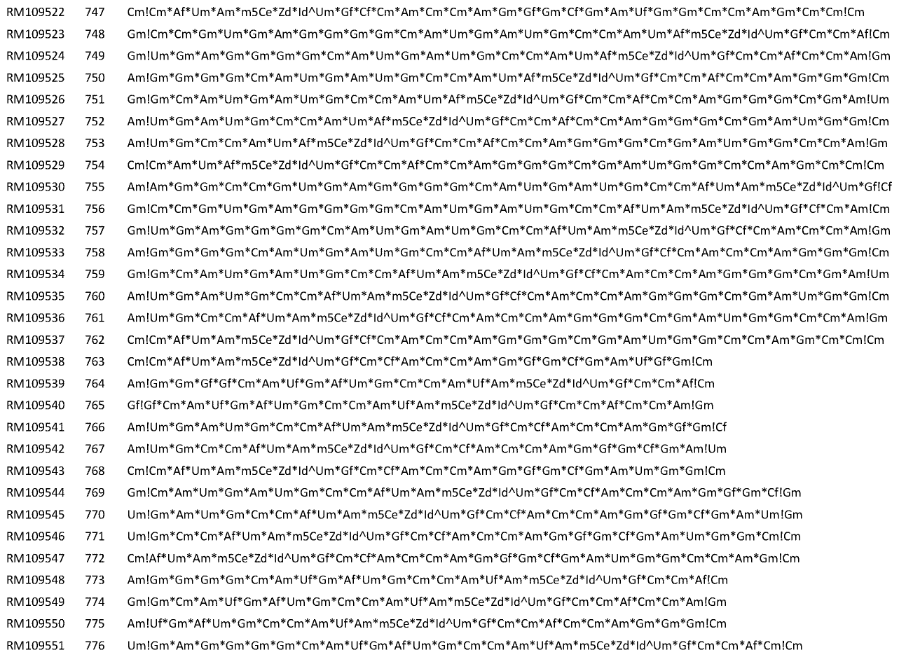

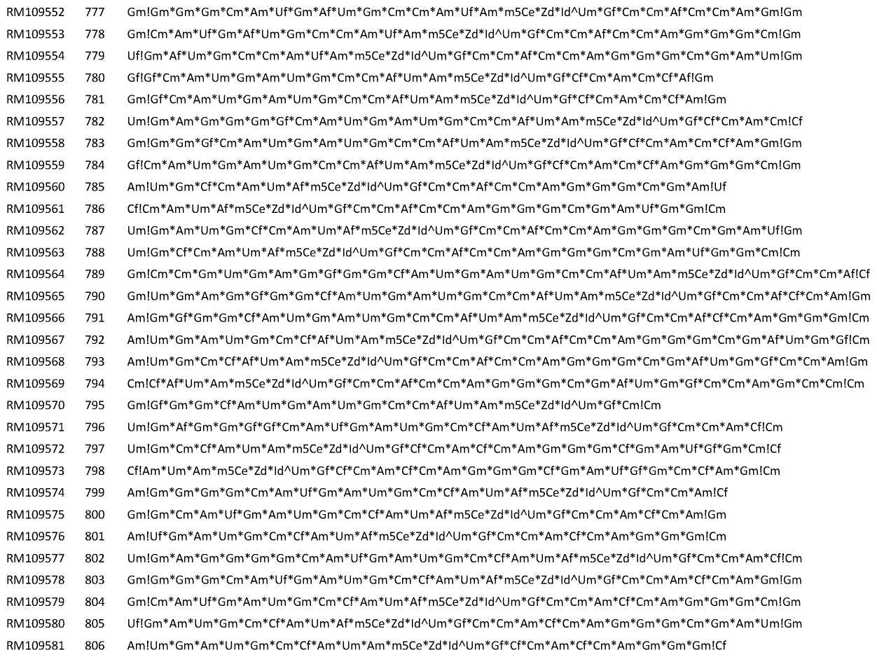

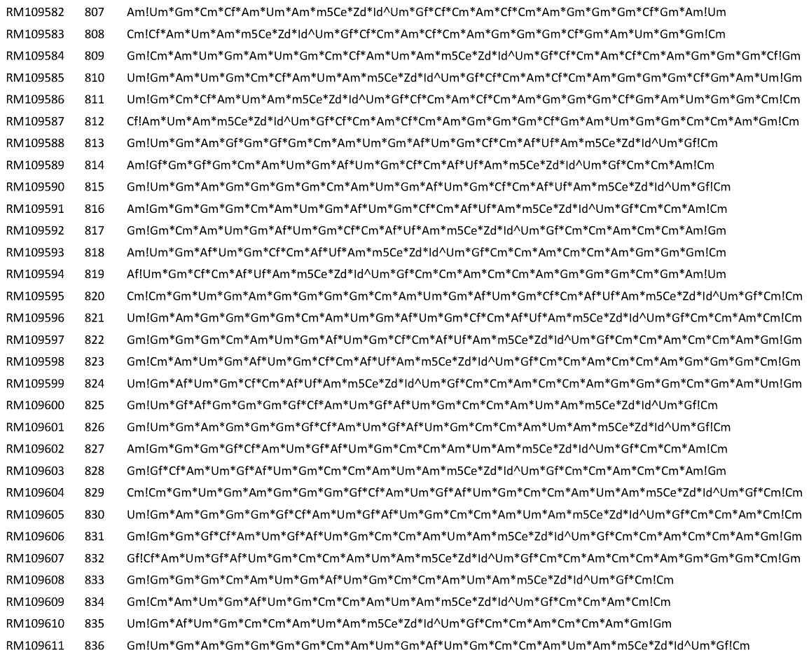

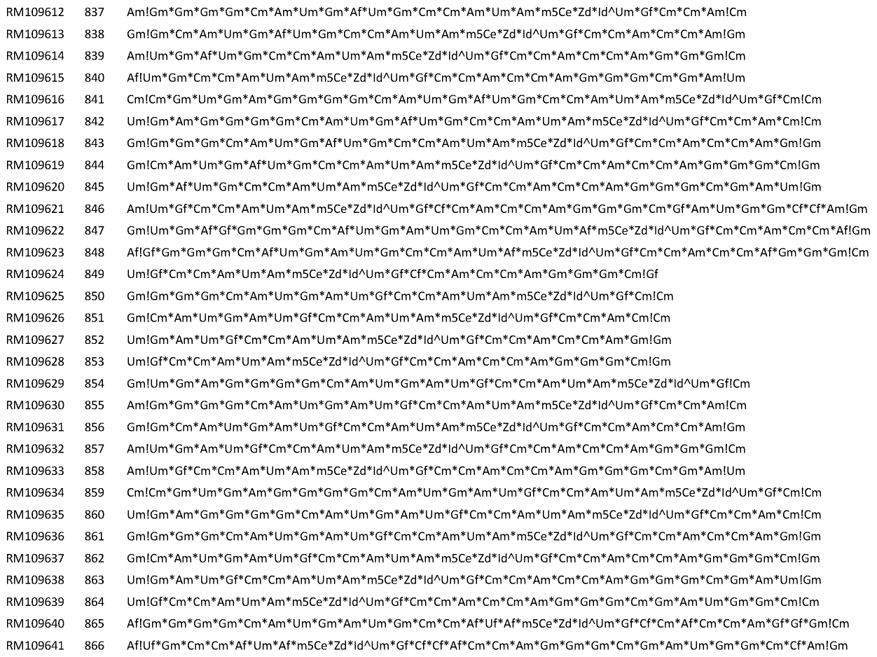

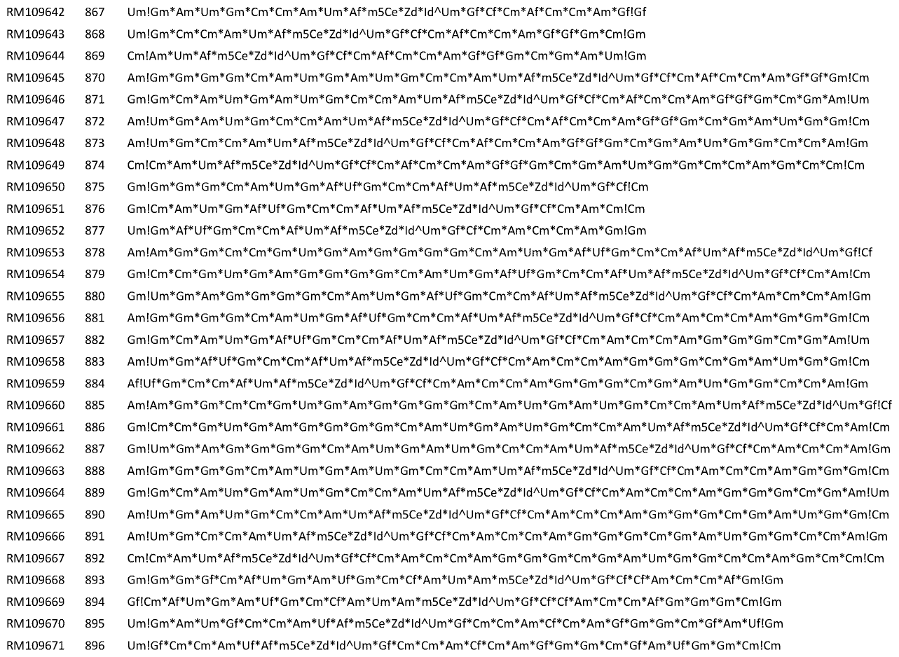

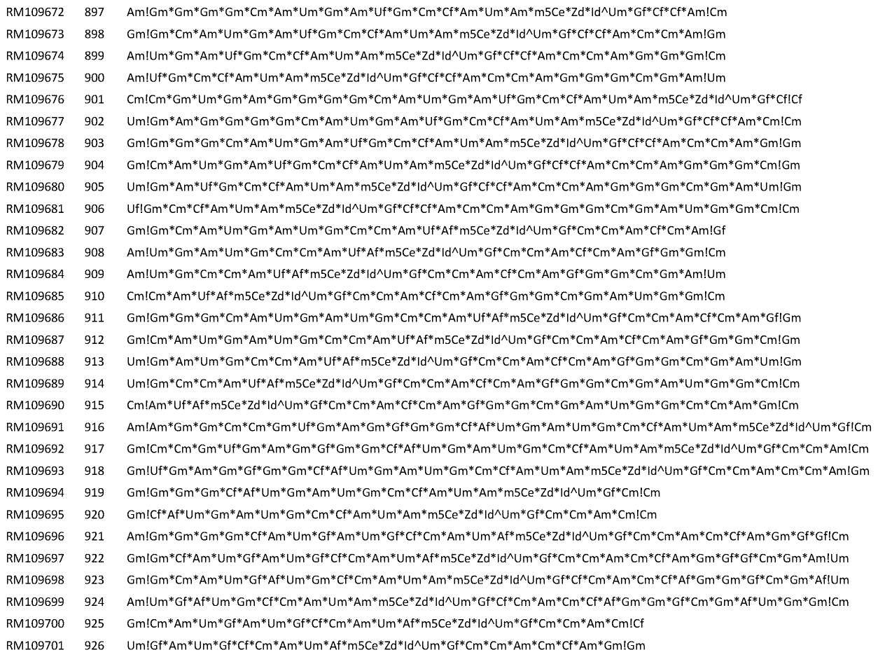

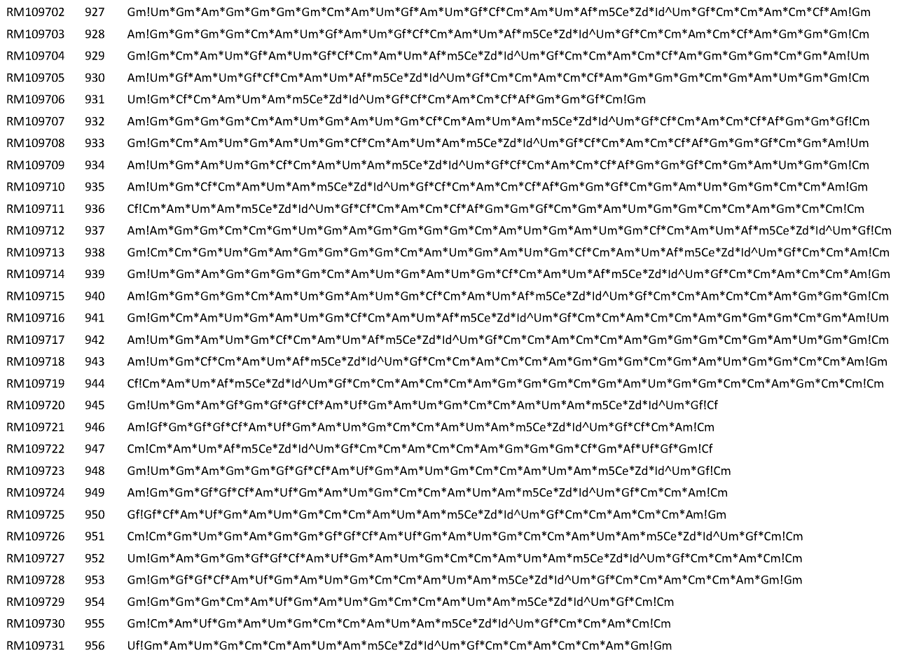

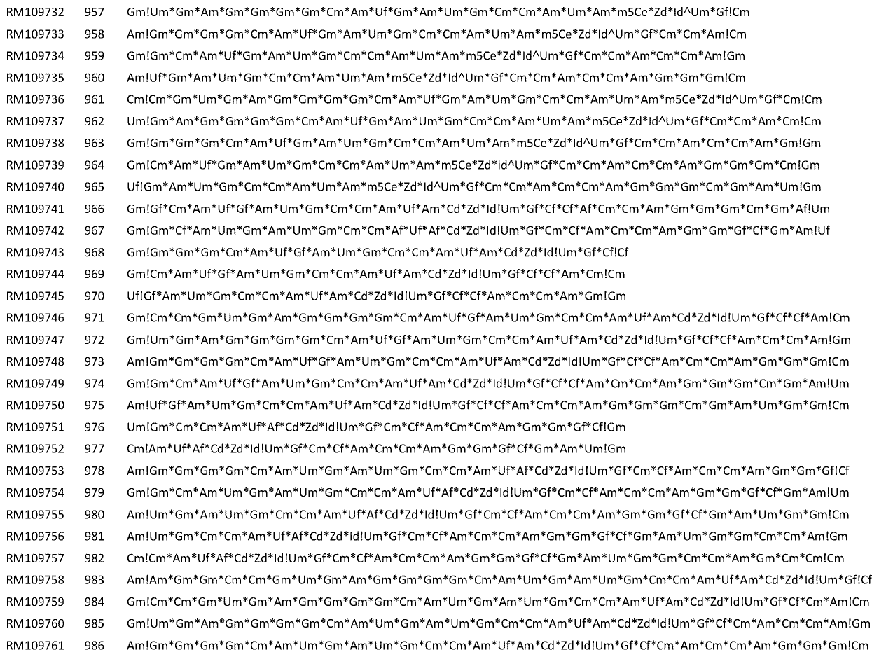

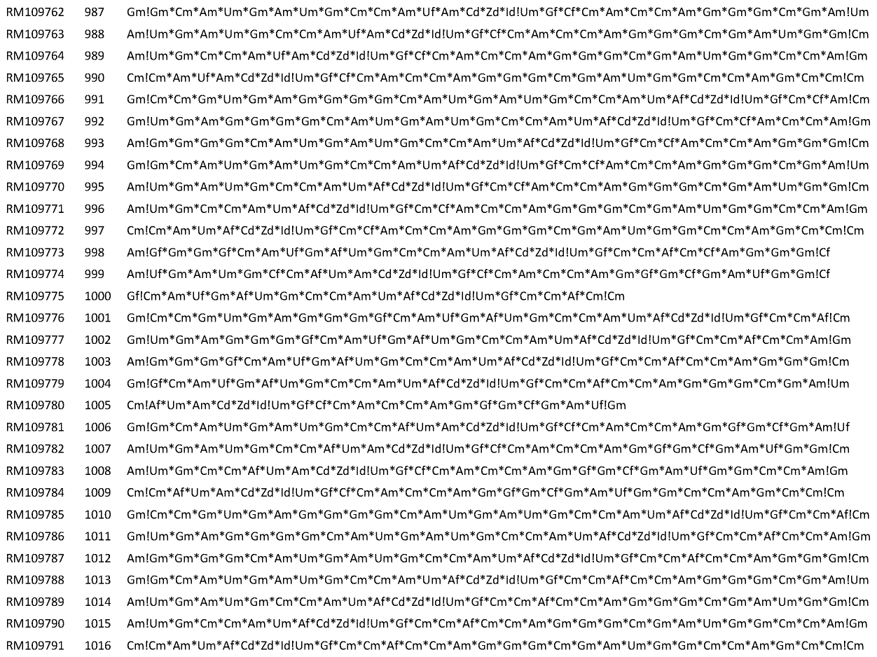

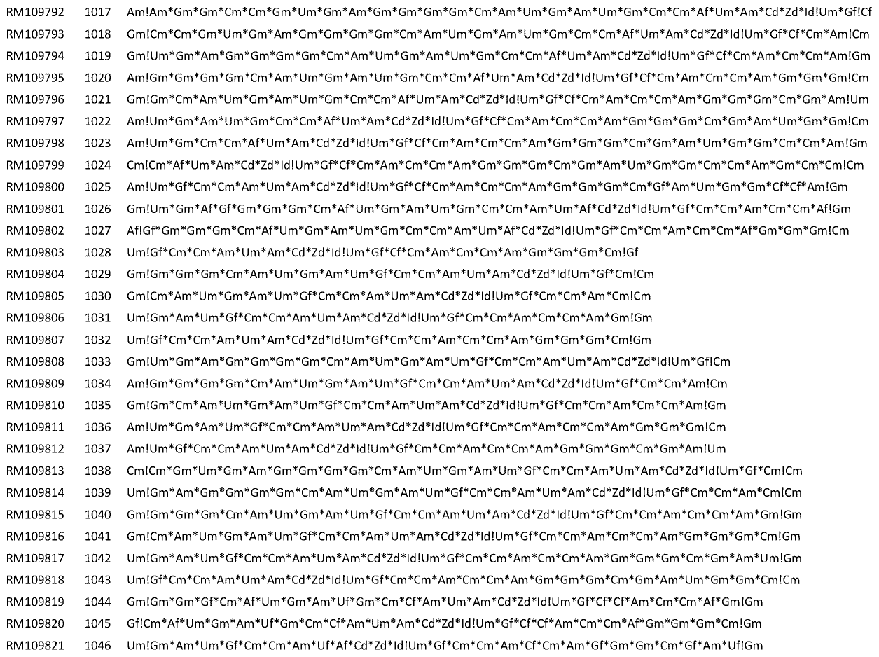

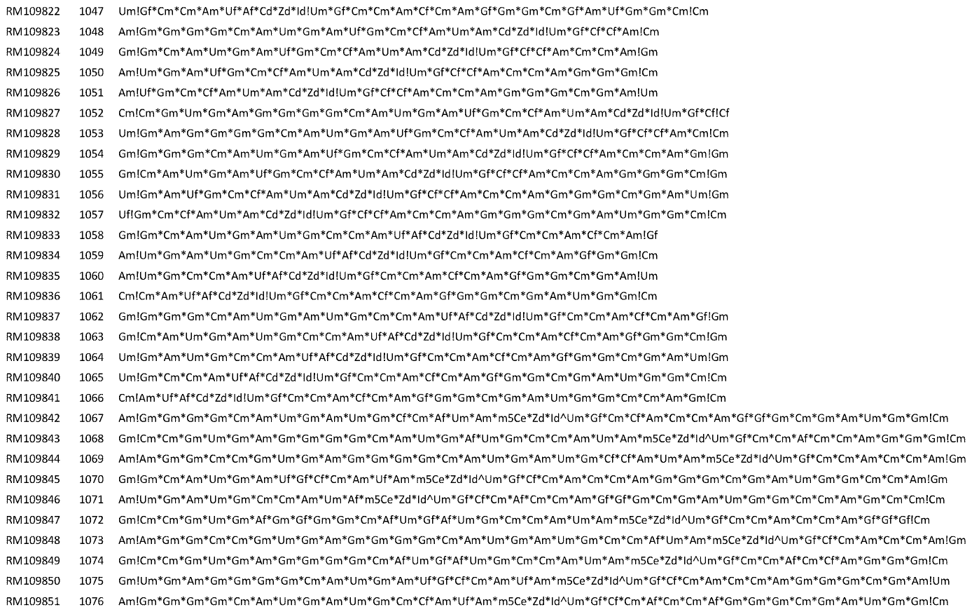

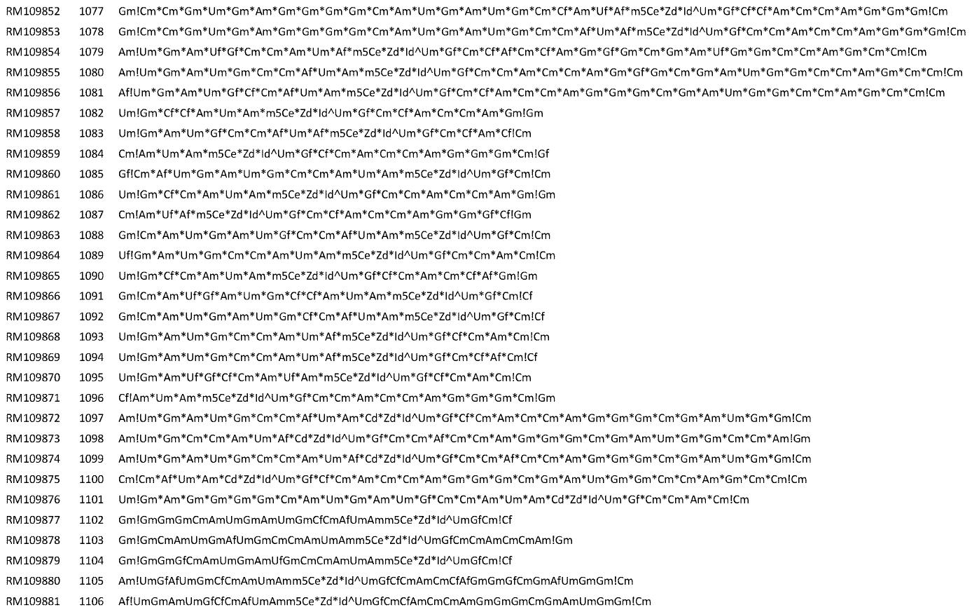

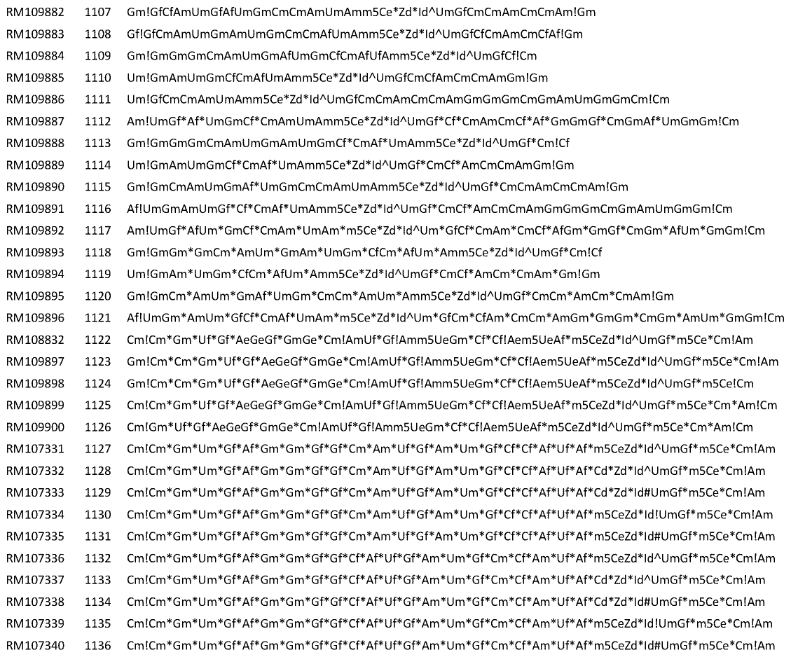

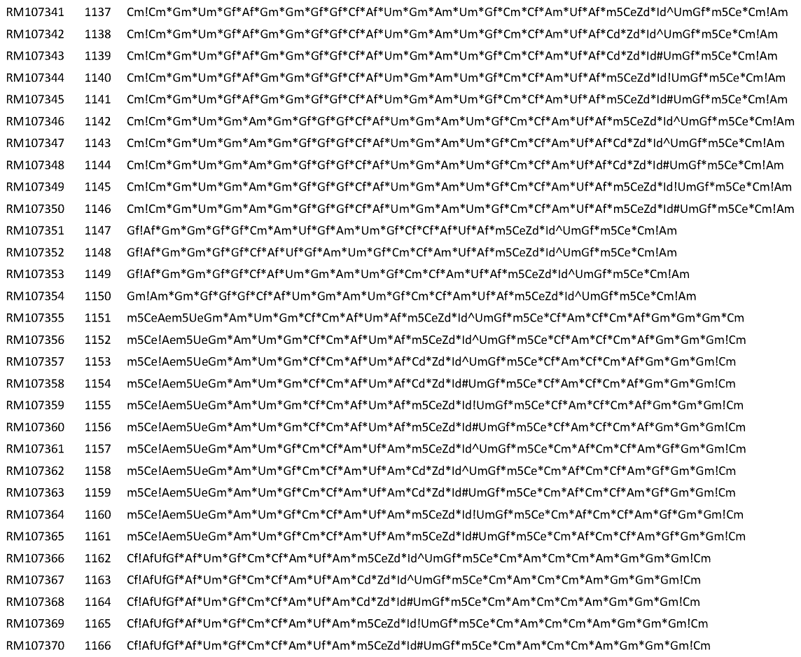

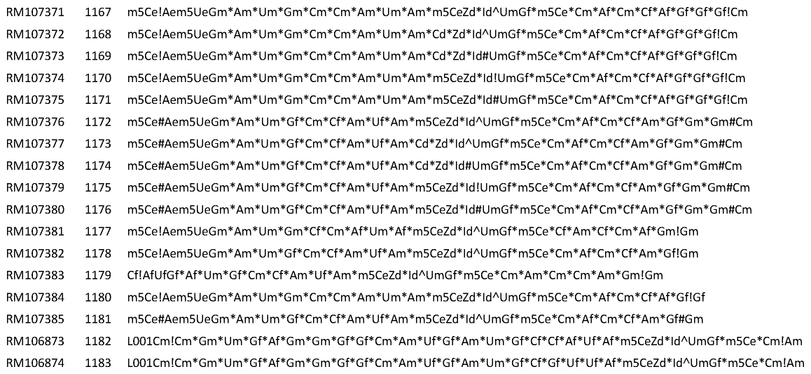

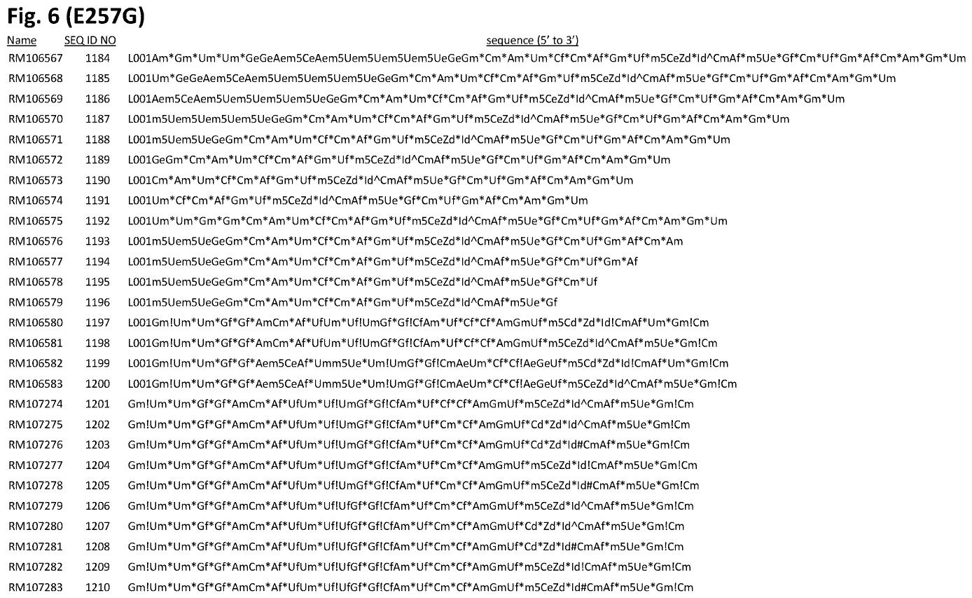

An initial set of 4×30 AONs were designed to target (separately) each of the four target adenosines in the human SLC10A1 transcript. The design and chemical modifications of these 120 AONs are provided in FIG. 1, FIG. 2, FIG. 3, and FIG. 4. Subsequently a large set of additional AONs was designed for targeting the adenosine in the CAG codon of SEQ ID NO: 1 coding for glutamine (Q) and to revert it to a CGG codon coding for arginine (R), thereby introducing the c.203A>G (Q68R) mutation. These additional AONs with their respective chemical modifications are provided in FIG. 5. Also, a large set of additional AONs were designed for targeting the adenosine in the GAG codon of SEQ ID NO:3 coding for glutamic acid (E) and to revert it to a GGG codon coding for glycine (G), thereby introducing the c.770A>G (E257G) mutation. These additional AONs with their respective chemical modifications are provided in FIG. 6.

For the initial screen of the AONs, the SLC10A1 mRNA expressing human hepatoblastoma cell line HuH-6 (Cell Lines Service) is transfected with 100 nM AON using Lipofectamine 2000 (Thermo Fisher Scientific) according to the manufacturer's specifications. Next to this, gymnotic exposure of the HuH-6 cells to different concentrations of the EONs with or without chemical agents is performed. After 72 hr of incubation, the medium is removed, and total RNA is isolated using the RNeasy Micro kit (Qiagen). cDNA is synthesized using Maxima Reverse Transcriptase kit using a mixture of random hexamers and oligo (dT) primers. The percentage of ADAR-mediated A-to-I conversion is determined by quantitative digital PCR (dPCR) assays designed to the different target sites, using the primers and probes provided in Table 1. The percentage is calculated by dividing the guanidine containing cDNA species by the total number of target copies, multiplied by 100. A control dPCR is performed upstream or downstream of the transcript for standardization purposes.

TABLE 1
Primers and probes with their respective sequences for
quantitative PCR assays. The primers and probes to
address exon 1 and exon 4 skip are also provided.
The 4 different targets are given by their amino acid
change. The SEQ ID NO of each primer or probe is given
between brackets following the respective sequence in
the middle column. The + indicates a Locked Nucleic
Acid (LNA) on the 3′ side of the symbol.

| Name | Sequence 5′-3′ | Item |
|---|---|---|
| hSLC10A1_e01_fw1 (Q68R) | GTTCAGCAAGATCAAGGCTCAC (125) | Primer |
| hSLC10A1_e01_edit(G)_FAM (Q68R) | CTGG+TGGCA+C+G+GTATGGCATCAT (126) | Probe |
| hSLC10A1_e01_orig(A)_HEX (Q68R) | CTGG+TGGCA+C+A+GTATGGCA+TCAT (127) | Probe |
| hSLC10A1_e01_TEX (Q68R) | CTGG+TGGCA+C+A+GTATGGCA+TCAT (128) | Probe |
| hSLC10A1_e02_rv1 (Q68R) | TGGAGCAGGTGGTCATCACAA (129) | Primer |
| hSLC10A1_e03_fw1 (E257G/Q261R) | TGACACCACTCTTGATTGCCAC (130) | Primer |
| hSLC10A1_e04_edit(G)_FAM2 (E257G) | TGG+ACATT+T+C+GGCATCCAGTCTCC (131) | Probe |
| hSLC10A1_e04_orig(A)_HEX2 (E257G) | TGG+ACATT+T+T+GGCATCCAGTC+TCC (132) | Probe |
| hSLC10A1_e04_TEX2 (E257G) | TGG+ACATT+T+T+GGCATCCAGTC+TCC (133) | Probe |
| hSLC10A1_e04_edit(G)_FAM3 (Q261R) | CAGT+C+C+CCATGCTGACAGTGCGT (134) | Probe |
| hSLC10A1_e04_orig(A)_HEX3 (Q261R) | CAGT+C+T+CCATGCTGACAG+TGCGT (135) | Probe |
| hSLC10A1_e04_TEX3 (Q261R) | CAGT+C+T+CCATGCTGACAG+TGCGT (136) | Probe |
| hSLC10A1_e04_rv1 (E257G/Q261R) | GAAAGGCCACATTGAGGATGGT (137) | Primer |
| hSLC10A1_e04_fw1 (K314E) | CTCAATGTGGCCTTTCCACCT (138) | Primer |
| hSLC10A1_e04-05_edit(G)_FAM4 (K314E) | TG+TTTTATC+C+T+C+GGGAGTCTTG+AA (139) | Probe |
| hSLC10A1_e04-05_orig(A)_HEX4 (K314E) | TG+TTTTATC+C+T+T+GGGAG+TCTTG+AA (140) | Probe |
| hSLC10A1_e04-05_TEX4 (K314E) | TG+TTTTATC+C+T+T+GGGAG+TCTTG+AA (141) | Probe |
| hSLC10A1_e05_rv1 (K314E) | CTTCAGTTGTGGCAGCTGTGTA (142) | Primer |
| hSLC10A1_e01_fw2 (exon 1 skip) | GGAGGGAACCTGTCCAATGT (143) | Primer |
| hSLC10A1_e01-02_CY5 (exon 1 skip) | CTCAG+CATTGTGATGACCACCTGC (144) | Probe |
| hSLC10A1_e01-02_TEX (exon 1 skip) | CTCAG+CATTGTGATGACCACCTGC (145) | Probe |
| hSLC10A1_e02_rv2 (exon 1 skip) | CTTGTCCTTCAGGTCCCCAT (146) | Primer |
| hSLC10A1_e03_fw2 (exon 4 skip) | ATTGCCACCTCCTCCCTGAT (147) | Primer |
| hSLC10A1_e03-05_CY5 (exon 4 skip) | C+TGCC+TCAA+TGGACG+ATAA+AACA+AA (148) | Probe |
| hSLC10A1_e05_rv2 (exon 4 skip) | GTCCTCCCCTTTGTAGGTGC (149) | Primer |

### Example 2. RNA Editing of Two Adenosines in the Human SLC10A1 Transcript in Primary Human Hepatocytes, Using a Saponin as Transfection Aid

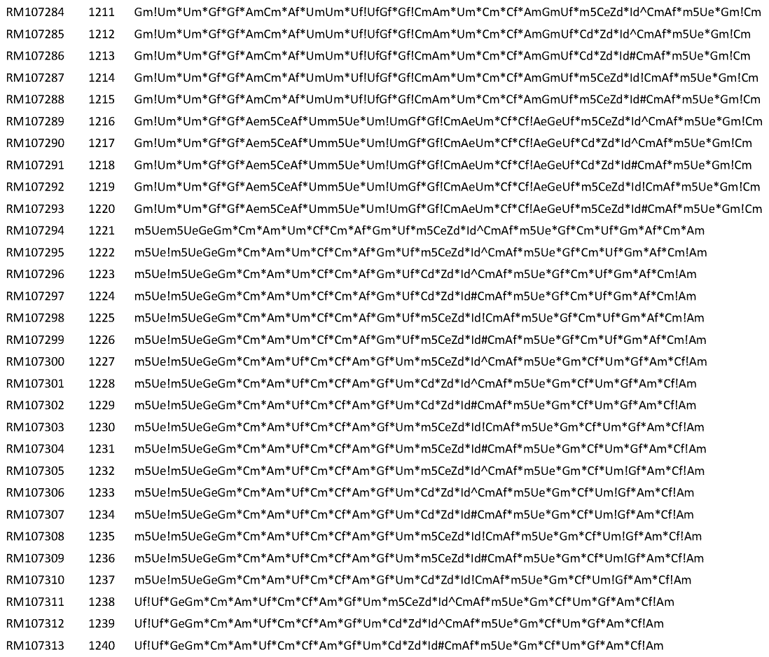

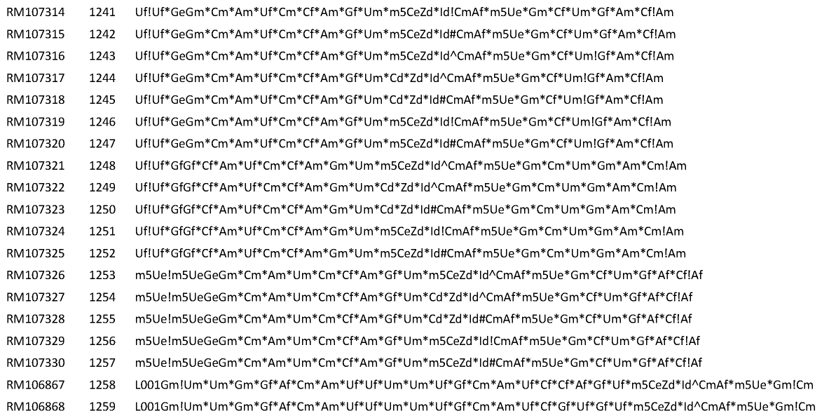

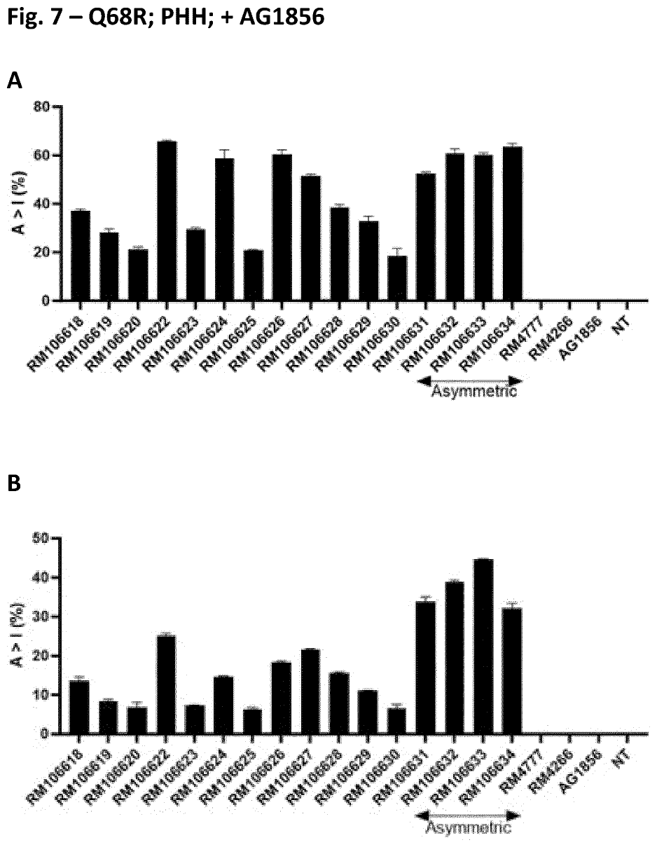

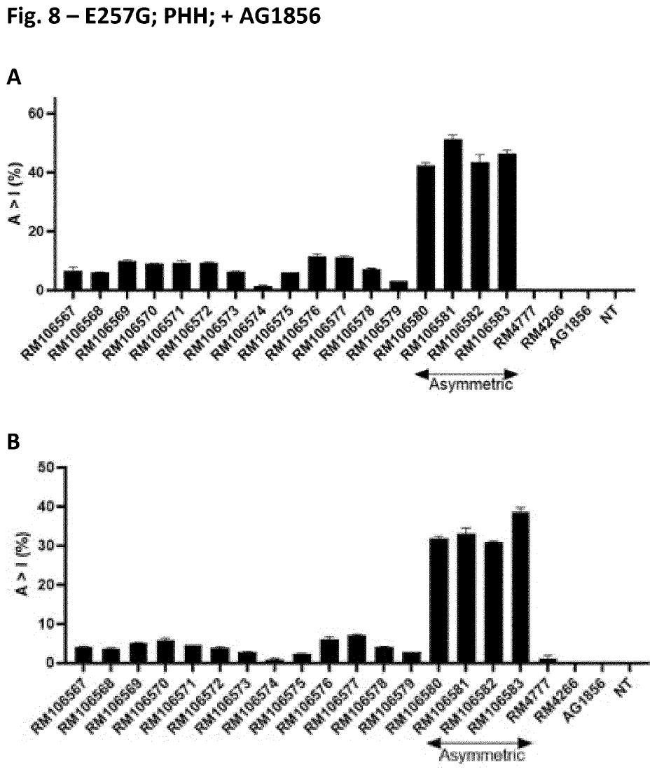

Primary human hepatocytes (PHH) were cultured and plated to ~700,000 cells per well and treated with 5 μM AON in the presence of 0.5 UM AG1856 for 72 hr. Then, the cells were harvested, and RNA was isolated as described above. Subsequently, a dPCR was performed with the respective primers and percentage editing was determined for a A>G change at position 203 of the human SLC10A1 mRNA (relating to the Q68R mutation in the protein) and for a A>G change at position 770 of the human SLC10A1 mRNA (relating to the E257G mutation in the protein). Both experiments were performed twice. FIG. 7A and FIG. 7B show the editing percentages that were measured in the two experiments separately for the c.203A>G editing (Q68R), using the 16 AONs (given in FIG. 5) as indicated. Although the percentages were slightly lower in the 2^{nd} experiment, editing percentages reached levels up to 65%, with RM106622, RM106624, RM106626, RM106631, RM106632, RM106633, and RM106634 performing best. FIG. 8A and FIG. 8B show the editing percentages that were measured in the two experiments separately for the c.770A>G editing (E257G), using the 17 AONs (given in FIG. 6) as indicated. Although the percentages were slightly lower in the 2^{nd} experiment, editing percentages reached levels up to 55%, with the 4 asymmetric AONs (wherein the length of the 5′ part of the AON calculated from the orphan position, in relation to the 3′ part of the AON, is much larger) RM106580, RM106581, RM106582, and RM106583 performing best, as indicated.

### Example 3. RNA Editing of Two Adenosines in the Human SLC10A1 Transcript in Primary Human Hepatocytes, Using Gymnotic Uptake

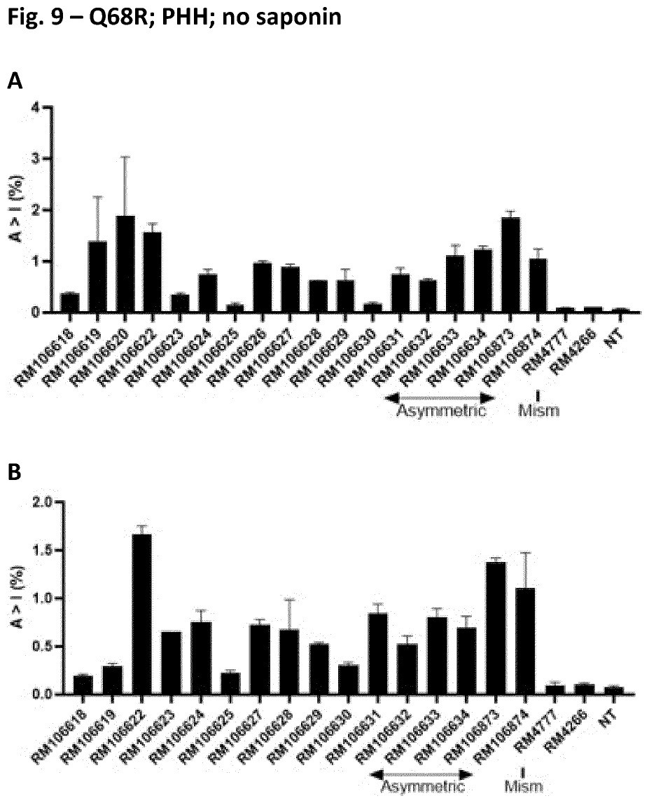

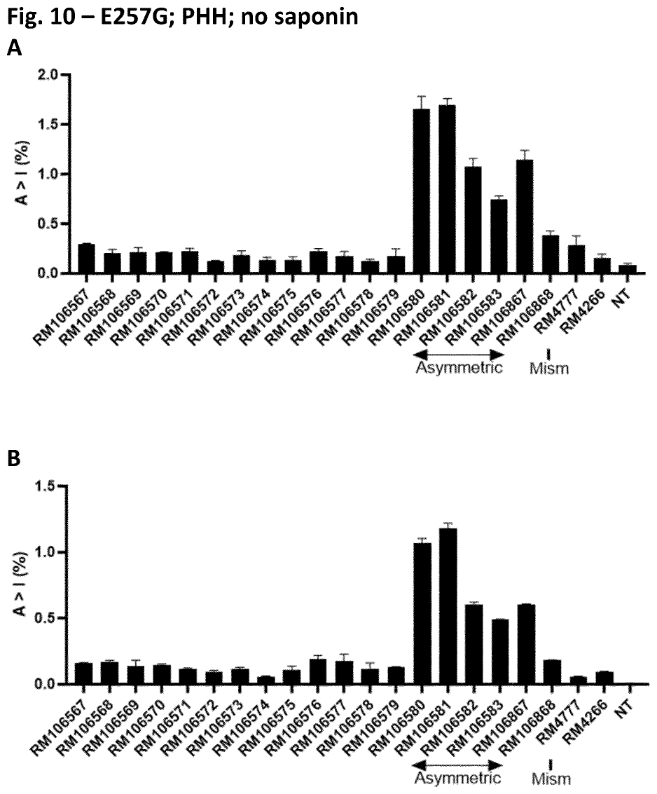

The experiments of Example 2 were repeated in PHH cells but without the aid of the AG1856 saponin during incubation. This entry of the oligonucleotide into the cells is also referred to as ‘gymnotic uptake’ or ‘gymnosis’. The incubation was again 72 hr, but with 10 UM AON and ~200,000 cells per well. In the case of c.203A>G (Q68R) a total of 18 different AONs was tested and in the case of c.770A>G (E257G) a total of 16 AONs was tested. FIG. 9A and FIG. 9B show the editing percentages that were measured in the two gymnotic uptake experiments separately for the c.203A>G editing (Q68R), using the 18 AONs (given in FIG. 5) as indicated. Although the percentages were again slightly lower in the 2^{nd} experiment, editing percentages reached levels up to 2%, with RM106620, RM106622, RM106873, and RM106874 performing best. FIG. 10A and FIG. 10B show the editing percentages that were measured in the two gymnotic uptake experiments separately for the c.770A>G editing (E257G), using the 16 AONs (given in FIG. 6) as indicated. Although also here the percentages were slightly lower in the 2^{nd} experiment, editing percentages reached levels up to 1.7%, with RM106867 and the 4 asymmetric AONs (wherein the length of the 5′ part of the AON calculated from the orphan position, in relation to the 3′ part of the AON, is much larger) RM106580, RM106581, RM106582, and RM106583 performing again significantly good, as indicated.

### Example 4. RNA Editing of Two Adenosines in the Human SLC10A1 Transcript in Liver Spheroids, Using Saponin-Supported Uptake Vs Gymnotic Uptake

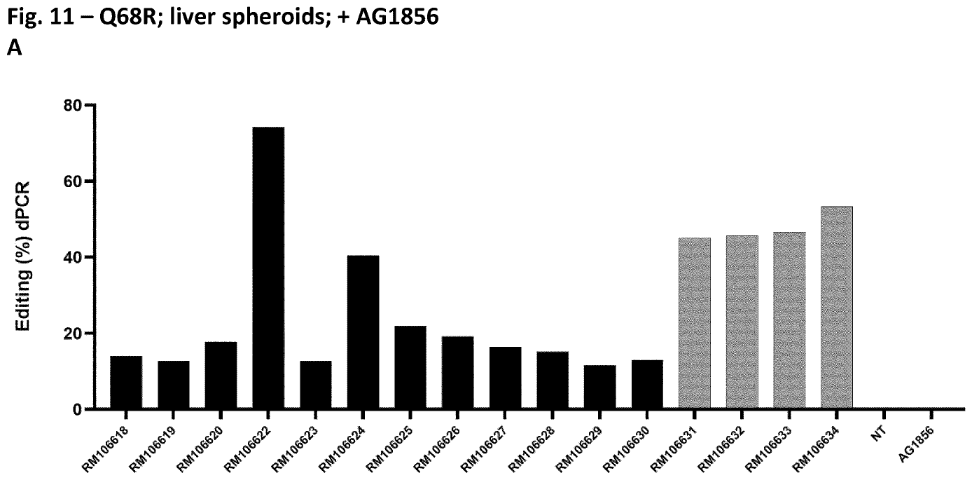

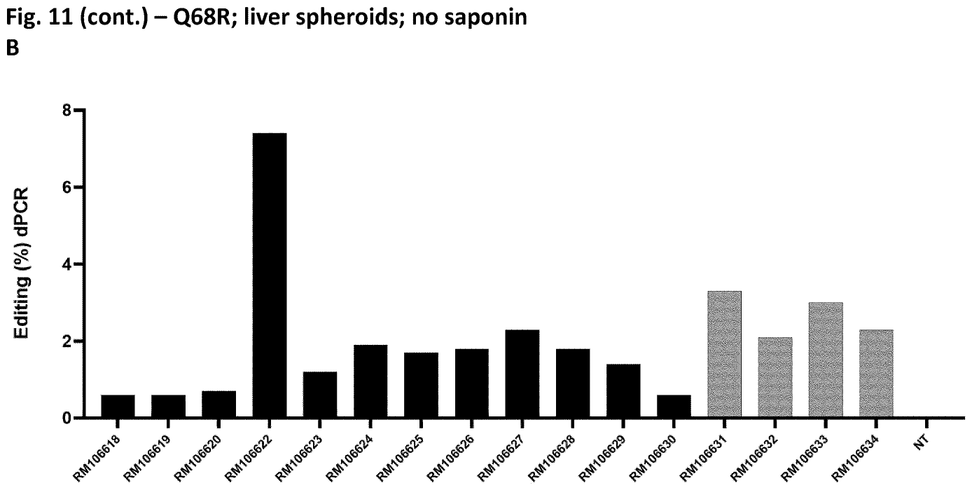

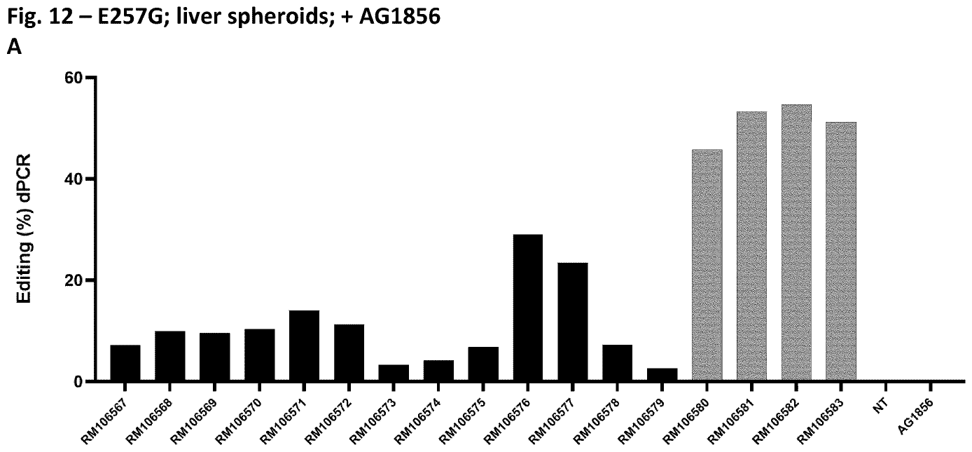

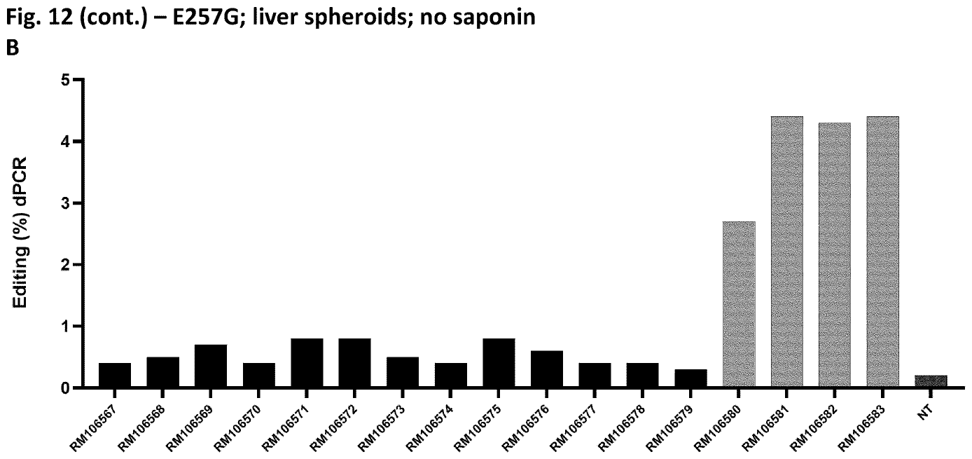

The experiments described in Examples 2 and 3 were repeated for both target sites but now with liver spheroid organoids generated from PHH cells using culturing conditions known to the person skilled in the art. Per incubation, 8 spheroids were used, that were incubated with 5 μM AON in each case, with or without 0.5 UM AG1856, for a period of 120 hr. RNA purification, cDNA generation, and dPCR were performed as outlined above. FIG. 11A and FIG. 11B show the editing percentages that were measured in the experiments in the presence of AG1856 and in the absence of saponin, respectively, in relation to the c.203A>G editing (Q68R), using the 16 AONs (given in FIG. 5) as indicated. Also in these liver organoids, editing percentages reached levels up to 75% in the presence of saponin, with RM106622, RM106873, RM10631, RM106632, RM106633, and RM106634 performing best, whereas RM106622 outperformed all other tested AONs when no saponin was applied, hence without any cell entry support, reaching levels of almost 8%. FIG. 12A and FIG. 12B show the editing percentages that were measured in the experiments in the presence of AG1856 and in the absence of saponin, respectively, in relation to the c.770A>G editing (E257G), using the 17 AONs (given in FIG. 5) as indicated. Also in these liver organoids, editing percentages reached levels up to 55% in the presence of saponin, with the ‘asymmetric’ AONs RM106580, RM106581, RM106582, and RM106583 performing best, also when no saponin was applied, reaching levels of almost 4.5%.

### Example 5. RNA Editing of Two Adenosines in the Human SLC10A1 Transcript in Human HepG2 Cells that Over-Express NTCP, Using Saponin-Supported Uptake

Human HepG2 cells that stably express wildtype human NTCP (HepG2NTCP) were cultured and plated to ~100,000 cells per well and treated with 5 μM AON in the presence of 0.5 UM AG1856 for 72 hr. Then, the cells were harvested, and RNA was isolated as described above. Subsequently, a dPCR was performed with the respective primers and percentage editing was determined for a A>G change at position 203 of the human SLC10A1 mRNA (relating to the Q68R mutation in the protein) and for a A>G change at position 770 of the human SLC10A1 mRNA (relating to the E257G mutation in the protein). Both experiments were performed twice. FIG. 13 shows the editing percentages that were measured for the c.203A>G editing (Q68R), using the 11 AONs (given in FIG. 5) as indicated. Editing percentages reached levels up to 25%, with RM106631, RM106632, RM106633, and RM106634 performing best. FIG. 14 shows the editing percentages that were measured for the c.770A>G editing (E257G), using the 10 AONs (given in FIG. 6) as indicated. Editing percentages reached levels up to 25%, with the 4 asymmetric AONs (wherein the length of the 5′ part of the AON calculated from the orphan position, in relation to the 3′ part of the AON, is much larger) RM106580, RM106581, RM106582, and RM106583 performing best, as indicated.

### Example 6. RNA Editing of Two Adenosines in the Human SLC10A1 Transcript in Primary Human Hepatocytes, Using Saponin-Supported Uptake

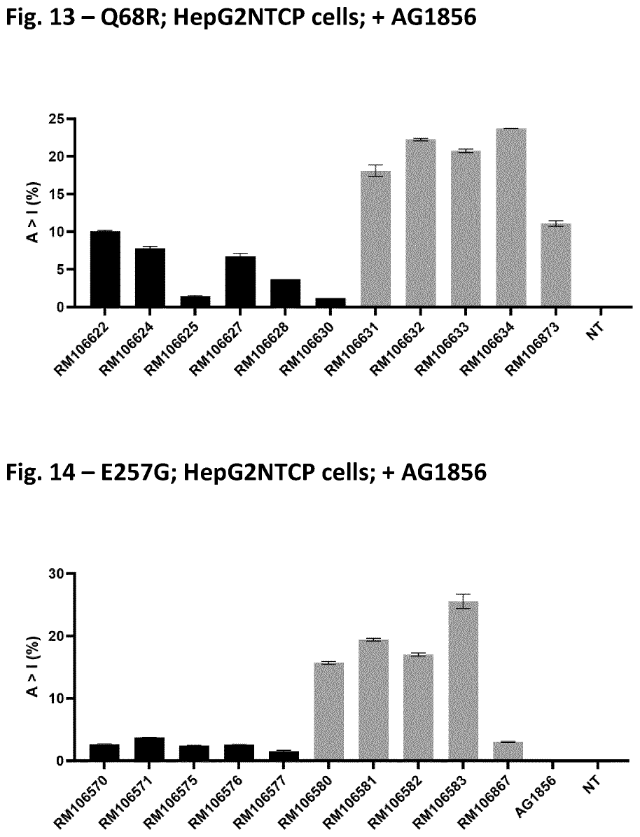

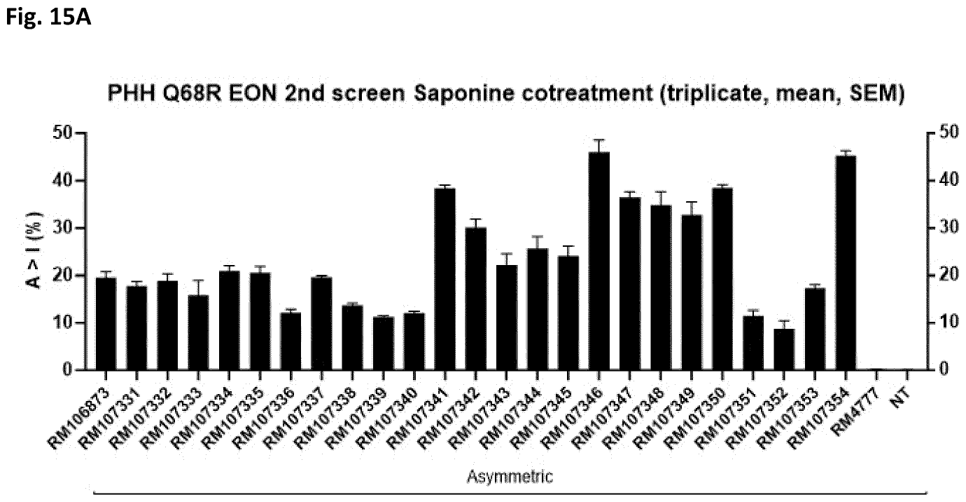

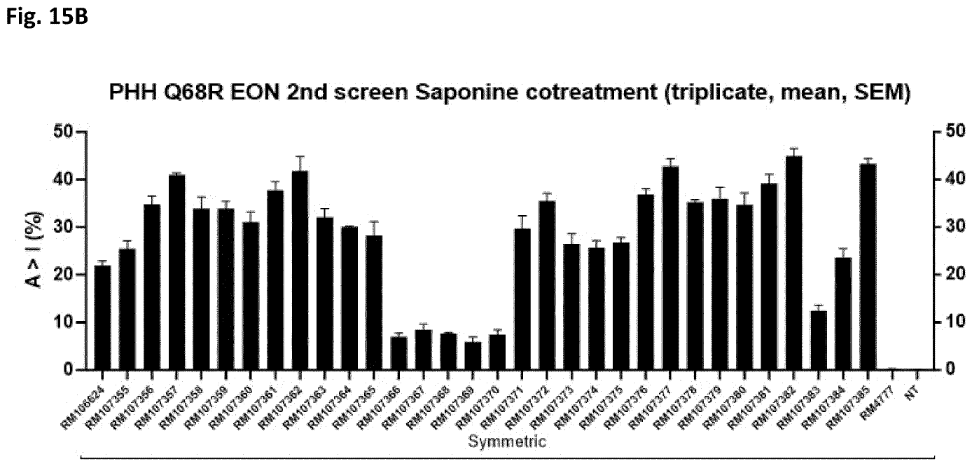

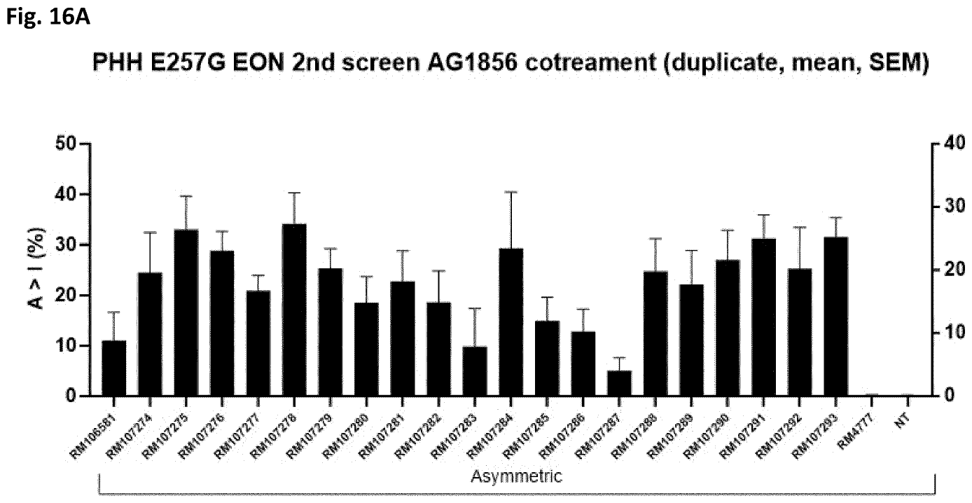

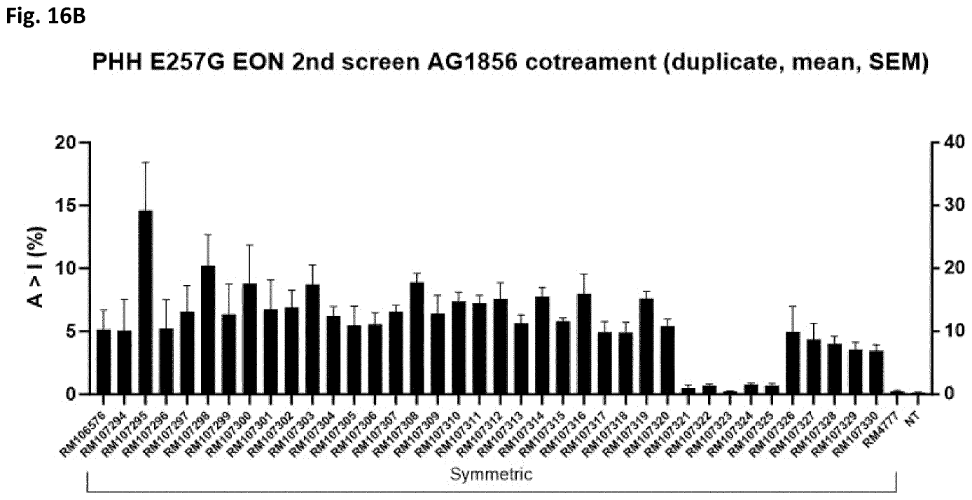

PHHs were cultured in a smaller format and plated to ~200,000 cells per well in a 12-well plate, and treated with 5 μM AON in the presence of 0.5 UM AG1856 for 72 hr. Then, the cells were harvested, and RNA was isolated as described above. Subsequently, a dPCR was performed with the respective primers and percentage editing was determined for a A>G change at position 203 of the human SLC10A1 mRNA (relating to the Q68R mutation in the protein) and for a A>G change at position 770 of the human SLC10A1 mRNA (relating to the E257G mutation in the protein). FIG. 15A shows the editing percentages that were measured for the c.203A>G editing (Q68R), using the 25 asymmetric AONs (given in FIG. 5) as indicated. Editing percentages reached levels up to 45%, with RM107341, RM107346, RM107350, and RM107354 performing best. FIG. 15B shows the editing percentages that were measured for the c.203A>G editing (Q68R), using the 32 symmetric AONs (given in FIG. 5) as indicated. Editing percentages reached levels up to 45%, with RM107357, RM107362, RM107377, RM107381, RM107382, and RM107385 performing best. FIG. 16A shows the editing percentages that were measured for the c.770A>G editing (E257G), using the 21 asymmetric AONs (given in FIG. 6) as indicated. Editing percentages reached levels up to 35%, with RM107275, RM107276, RM107278, RM107284, RM107291, and RM107293 performing best. FIG. 16B shows the editing percentages that were measured for the c.770A>G editing (E257G), using the 38 symmetric AONs (given in FIG. 6) as indicated. Editing percentages reached levels up to 15%, with RM107295 and RM107298 performing best, as indicated. These experiments appear to confirm the earlier findings above that the c.203A>G editing (Q68R) is more efficient with symmetric AONs, whereas the c.770A>G editing (E257G) is more efficient with asymmetric AONs.

### Example 7. Expression of Loss-of-Function Mutants of NTCP on the Cell Membrane of U2OS Cells after Transfection

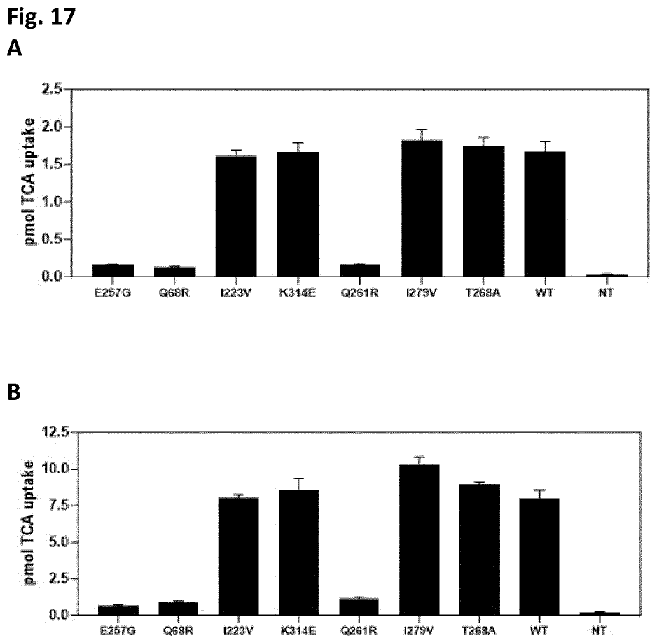

To test whether the envisioned NTCP mutants as outlined herein indeed resulted in loss of function in bile transport over the cell membrane, human U2OS cells were cultured in 48-wells plates and left to adhere overnight. The next day, cells were transfected with either a plasmid encoding the wildtype human NTCP protein (as a positive control) and plasmids encoding seven mutant forms of human NTCP: E257G, Q68R, 1223V, K314E, Q261R, 1279V, and T268A. The experiment was performed in triplicate. Upon transfection, the cells were treated with either 1 μM or 10 μM radiolabelled taurocholic acid (TCA) for 48 hr. TCA is a human primary bile acid. After TCA treatment, the bile acid and medium were washed away and the cells were washed several times, after which the radioactivity in each well was determined, which is a measure of TCA uptake in these cells. FIG. 17A and FIG. 17B show the pmol TCA uptake in the different cells treated with the different expression plasmids, wherein FIG. 17A shows the results with the 1 μM treatment and FIG. 17B shows the results with the 10 UM treatment. No bile acid uptake was observed when the cells were not transfected, indicating an absence of the NTCP protein in U2OS cells. The ‘normal’ level of TCA uptake using the wildtype NTCP expression plasmid was approximately 1.6% and 7.6%, respectively. These levels were also reached with the mutants 1223V, K314E, 1279V, and T268A. However, a significant absence in bile acid uptake was observed after expressing the mutants E257G, Q68R, and Q261R, showing the rationale behind introducing one or more of these mutations in the human NTCP transcript to therethrough lower the ability to transport bile acid across the cell membrane in liver cells.

### Example 8. C.203A>G Editing (Q68R) in SLC10A1 Transcripts in Primary Human Hepatocytes in a High-Throughput Screen

Subsequently, a set of 960 EONs was designed (EON numbers RM108942 to RM109900; represented by SEQ ID NO: 167 to 1126, see FIG. 5) comprising a variety of chemical and other modifications and all targeting the human SLC10A1 transcript at position c.203A (Q68R), as discussed herein. These EONs were tested as follows: On day 0, PHHs (5.0×10⁴ cells/well) were transfected with EONs, in triplicates, using Lipofectamine® RNAiMAX Reagent at the same time of seeding, following the protocol of the manufacturer. The plates containing cells, medium and EON were held at 37° C., 5% CO₂ for 72 hrs, during which the medium was refreshed 24 hrs after transfection/plating.

On day 3 (72 hrs post transfection/plating) the supernatants were discarded, and subsequent analysis was performed as follows. Cells were collected and used for RNA isolation using a RNeasy 96 Kit (Qiagen-74182) according to the manufacturer's instructions. Extracted RNA was treated with DNasel (ThermoFisher-EN0521) according to manufacturer's protocol. Samples were incubated at 37° C. for 30 min and then 1 μL 50 mM EDTA was added and further incubated at 60° C. for 2 min. The total RNAs were then reverse-transcribed using the Maxima Reverse Transcriptase (Thermo-EP0742) kit with oligo-dT primer, random Hexamer Primer, and dNTP Mix (10 mM each). A quantitative PCR was then performed with the Digital PCR System (Bio-Rad, QX200) in 22 μl aliquots of reaction mixtures containing cDNA, appropriate pairs of primers and ddPCR Supermix for Probes (no dUTP) (Bio-Rad-1863024). The primers for this Q68R target (Table 1) were used with a PCR program that was as follows: 10 min at 95° C.; 40×30 sec at 94° C. and 60 sec at 63° C., 10 min at 98° C. and a hold step at 4° C. Then the plate was placed into the droplet QX200 reader to measure the number of positive droplets. The editing percentage was calculated by pooling the three replicates for each transfection for all A and G counts and then scored as follows:

$$score = SUM ( G ) / ( SUM ( A + G ) * 100$$

The p-value (p=0.05) represents the probability of 3 treated replicates to be different from the 3 non-treated replicates. Editing scores of the EONs are ranked from the highest editing percentage to the lowest. From the 960 EONs that were tested, 415 EONs scored above 0% editing. These scores are provided below in Table 2. The remaining 545 EONs all gave 0% editing and are not listed in Table 2. Unexpectedly, some EONs performed significantly good, with RM109899, RM108970, RM109900, RM109571, RM108832, RM109040, RM109898, RM109306, and RM108977 performing best, all with percentages above 12% and with some replicates going above 21%.

TABLE 2
Editing percentages of EONs targeting c.203A
(Q68R) in HPPs, after transfection.

| EON | Editing % |
|---|---|
| RM109899 | 21.83 |
| RM108970 | 21.64 |
| RM109900 | 21.36 |
| RM109571 | 18.58 |
| RM108832 | 16.62 |
| RM109040 | 14.91 |
| RM109898 | 13.10 |
| RM109306 | 12.83 |
| RM108977 | 12.02 |
| RM109873 | 11.69 |
| RM108984 | 11.66 |
| RM108973 | 11.64 |
| RM108983 | 11.62 |
| RM109692 | 11.60 |
| RM109822 | 11.09 |
| RM109693 | 10.84 |
| RM109323 | 10.73 |
| RM109577 | 10.68 |
| RM108999 | 10.39 |
| RM109317 | 10.33 |
| RM109231 | 9.75 |
| RM109689 | 9.72 |
| RM108986 | 9.71 |
| RM109559 | 9.69 |
| RM108972 | 9.65 |
| RM109235 | 9.52 |
| RM109015 | 9.46 |
| RM109324 | 9.40 |
| RM109404 | 9.17 |
| RM109227 | 9.06 |
| RM109835 | 8.97 |
| RM109567 | 8.92 |
| RM109563 | 8.71 |
| RM109325 | 8.68 |
| RM109228 | 8.59 |
| RM109447 | 8.51 |
| RM109269 | 8.35 |
| RM109225 | 8.19 |
| RM109528 | 7.86 |
| RM109838 | 7.83 |
| RM108979 | 7.83 |
| RM108987 | 7.82 |
| RM109020 | 7.75 |
| RM109386 | 7.72 |
| RM109370 | 7.71 |
| RM109239 | 7.71 |
| RM108985 | 7.70 |
| RM109703 | 7.67 |
| RM109874 | 7.63 |
| RM109853 | 7.61 |
| RM109821 | 7.55 |
| RM109367 | 7.50 |
| RM109526 | 7.48 |
| RM108998 | 7.46 |
| RM109705 | 7.44 |
| RM109387 | 7.37 |
| RM109852 | 7.33 |
| RM109320 | 7.15 |
| RM109380 | 7.12 |
| RM109702 | 7.08 |
| RM109448 | 7.04 |
| RM109388 | 6.99 |
| RM109560 | 6.96 |
| RM109107 | 6.93 |
| RM109707 | 6.92 |
| RM108976 | 6.82 |
| RM109843 | 6.80 |
| RM109854 | 6.80 |
| RM109036 | 6.78 |
| RM109619 | 6.78 |
| RM109659 | 6.76 |
| RM109471 | 6.71 |
| RM109400 | 6.70 |
| RM109405 | 6.62 |
| RM109290 | 6.53 |
| RM109450 | 6.52 |
| RM109118 | 6.48 |
| RM109411 | 6.45 |
| RM109516 | 6.42 |
| RM109769 | 6.41 |
| RM109580 | 6.40 |
| RM109410 | 6.38 |
| RM109469 | 6.36 |
| RM109446 | 6.32 |
| RM109475 | 6.23 |
| RM109545 | 6.17 |
| RM109492 | 6.14 |
| RM109511 | 6.07 |
| RM109422 | 6.07 |
| RM109507 | 6.04 |
| RM109505 | 6.03 |
| RM109174 | 6.00 |
| RM109420 | 5.98 |
| RM109402 | 5.97 |
| RM109291 | 5.95 |
| RM109230 | 5.89 |
| RM109328 | 5.86 |
| RM109077 | 5.80 |
| RM109007 | 5.79 |
| RM109697 | 5.79 |
| RM109332 | 5.78 |
| RM109704 | 5.73 |
| RM109378 | 5.73 |
| RM109839 | 5.72 |
| RM109790 | 5.71 |
| RM109558 | 5.62 |
| RM109371 | 5.59 |
| RM109670 | 5.55 |
| RM109403 | 5.55 |
| RM109513 | 5.49 |
| RM109233 | 5.49 |
| RM109527 | 5.49 |
| RM109770 | 5.42 |
| RM108966 | 5.36 |
| RM109067 | 5.36 |
| RM109390 | 5.36 |
| RM108981 | 5.35 |
| RM109103 | 5.33 |
| RM109237 | 5.33 |
| RM109454 | 5.33 |
| RM108991 | 5.32 |
| RM109418 | 5.31 |
| RM109140 | 5.28 |
| RM109106 | 5.27 |
| RM109755 | 5.27 |
| RM109666 | 5.25 |
| RM109840 | 5.20 |
| RM109463 | 5.19 |
| RM109438 | 5.18 |
| RM109413 | 5.17 |
| RM109282 | 5.17 |
| RM109756 | 5.15 |
| RM108975 | 5.12 |
| RM109788 | 5.11 |
| RM109080 | 5.10 |
| RM109002 | 5.09 |
| RM109517 | 5.06 |
| RM109622 | 5.03 |
| RM109372 | 4.99 |
| RM109846 | 4.99 |
| RM109771 | 4.98 |
| RM109599 | 4.98 |
| RM109065 | 4.95 |
| RM109816 | 4.93 |
| RM109458 | 4.90 |
| RM108971 | 4.89 |
| RM109715 | 4.87 |
| RM109329 | 4.86 |
| RM109820 | 4.84 |
| RM109138 | 4.82 |
| RM109085 | 4.81 |
| RM109086 | 4.78 |
| RM109331 | 4.72 |
| RM109018 | 4.69 |
| RM109229 | 4.68 |
| RM109584 | 4.67 |
| RM109830 | 4.66 |
| RM109455 | 4.65 |
| RM109643 | 4.65 |
| RM109753 | 4.63 |
| RM109347 | 4.61 |
| RM109098 | 4.59 |
| RM109322 | 4.59 |
| RM109679 | 4.59 |
| RM109461 | 4.56 |
| RM109453 | 4.53 |
| RM109289 | 4.50 |
| RM109544 | 4.49 |
| RM109238 | 4.43 |
| RM109710 | 4.38 |
| RM109596 | 4.37 |
| RM109172 | 4.36 |
| RM109313 | 4.33 |
| RM109141 | 4.32 |
| RM108995 | 4.30 |
| RM109173 | 4.26 |
| RM108980 | 4.24 |
| RM109828 | 4.23 |
| RM109432 | 4.17 |
| RM109360 | 4.11 |
| RM109473 | 4.06 |
| RM109054 | 4.06 |
| RM109304 | 4.06 |
| RM109352 | 4.05 |
| RM109416 | 4.05 |
| RM109428 | 4.05 |
| RM109876 | 4.03 |
| RM109565 | 4.02 |
| RM109017 | 3.99 |
| RM109706 | 3.99 |
| RM109709 | 3.91 |
| RM109655 | 3.90 |
| RM109008 | 3.88 |
| RM108997 | 3.87 |
| RM109376 | 3.87 |
| RM109585 | 3.86 |
| RM109718 | 3.86 |
| RM109564 | 3.85 |
| RM109439 | 3.82 |
| RM109524 | 3.82 |
| RM109779 | 3.81 |
| RM109684 | 3.80 |
| RM109739 | 3.78 |
| RM109297 | 3.78 |
| RM109364 | 3.76 |
| RM109551 | 3.75 |
| RM109826 | 3.75 |
| RM109460 | 3.73 |
| RM109789 | 3.73 |
| RM109160 | 3.73 |
| RM109462 | 3.71 |
| RM109427 | 3.71 |
| RM109680 | 3.71 |
| RM109698 | 3.70 |
| RM109010 | 3.69 |
| RM109276 | 3.69 |
| RM109775 | 3.66 |
| RM109186 | 3.62 |
| RM109637 | 3.61 |
| RM109348 | 3.61 |
| RM109831 | 3.60 |
| RM109468 | 3.60 |
| RM109607 | 3.58 |
| RM109245 | 3.58 |
| RM109504 | 3.57 |
| RM109783 | 3.56 |
| RM109651 | 3.55 |
| RM109415 | 3.53 |
| RM108978 | 3.53 |
| RM109424 | 3.52 |
| RM109423 | 3.50 |
| RM109206 | 3.50 |
| RM109056 | 3.49 |
| RM109287 | 3.40 |
| RM109383 | 3.39 |
| RM109445 | 3.38 |
| RM109222 | 3.37 |
| RM109342 | 3.35 |
| RM109226 | 3.34 |
| RM109594 | 3.34 |
| RM109109 | 3.34 |
| RM109512 | 3.32 |
| RM109381 | 3.31 |
| RM109675 | 3.30 |
| RM109292 | 3.28 |
| RM109819 | 3.26 |
| RM109084 | 3.24 |
| RM109155 | 3.24 |
| RM109449 | 3.24 |
| RM109074 | 3.22 |
| RM109574 | 3.20 |
| RM109742 | 3.20 |
| RM109444 | 3.18 |
| RM109754 | 3.15 |
| RM109440 | 3.14 |
| RM109307 | 3.11 |
| RM109161 | 3.08 |
| RM109764 | 3.04 |
| RM109496 | 3.03 |
| RM109272 | 3.02 |
| RM109506 | 3.00 |
| RM109641 | 2.98 |
| RM109207 | 2.97 |
| RM109459 | 2.95 |
| RM109582 | 2.92 |
| RM109262 | 2.91 |
| RM109303 | 2.90 |
| RM109202 | 2.89 |
| RM109508 | 2.89 |
| RM109700 | 2.88 |
| RM109817 | 2.84 |
| RM109094 | 2.81 |
| RM108968 | 2.81 |
| RM109441 | 2.80 |
| RM108989 | 2.79 |
| RM109047 | 2.79 |
| RM109126 | 2.79 |
| RM109366 | 2.78 |
| RM109257 | 2.72 |
| RM109708 | 2.69 |
| RM109034 | 2.69 |
| RM108990 | 2.68 |
| RM109502 | 2.67 |
| RM109812 | 2.66 |
| RM109108 | 2.66 |
| RM109234 | 2.65 |
| RM109279 | 2.62 |
| RM109683 | 2.61 |
| RM109615 | 2.60 |
| RM109829 | 2.60 |
| RM109326 | 2.58 |
| RM109263 | 2.58 |
| RM109149 | 2.55 |
| RM109476 | 2.54 |
| RM109192 | 2.54 |
| RM109031 | 2.53 |
| RM109006 | 2.53 |
| RM109763 | 2.50 |
| RM109357 | 2.48 |
| RM109494 | 2.47 |
| RM109442 | 2.46 |
| RM109609 | 2.46 |
| RM109740 | 2.43 |
| RM109681 | 2.41 |
| RM108943 | 2.41 |
| RM109836 | 2.41 |
| RM109171 | 2.39 |
| RM109665 | 2.38 |
| RM109623 | 2.38 |
| RM109657 | 2.35 |
| RM109509 | 2.35 |
| RM109133 | 2.34 |
| RM109762 | 2.31 |
| RM109205 | 2.31 |
| RM109650 | 2.29 |
| RM109335 | 2.26 |
| RM109728 | 2.25 |
| RM109727 | 2.24 |
| RM109046 | 2.23 |
| RM109327 | 2.23 |
| RM109333 | 2.21 |
| RM109701 | 2.20 |
| RM109576 | 2.20 |
| RM108956 | 2.19 |
| RM109805 | 2.15 |
| RM109807 | 2.15 |
| RM109009 | 2.13 |
| RM109073 | 2.11 |
| RM109081 | 2.11 |
| RM109556 | 2.10 |
| RM109346 | 2.08 |
| RM109377 | 2.07 |
| RM109833 | 2.04 |
| RM109798 | 2.03 |
| RM109419 | 2.02 |
| RM109869 | 2.01 |
| RM109385 | 1.99 |
| RM109349 | 1.99 |
| RM109397 | 1.96 |
| RM109296 | 1.94 |
| RM109213 | 1.94 |
| RM109354 | 1.90 |
| RM109215 | 1.90 |
| RM109066 | 1.90 |
| RM109575 | 1.90 |
| RM109470 | 1.89 |
| RM109308 | 1.88 |
| RM109003 | 1.87 |
| RM109099 | 1.87 |
| RM109818 | 1.87 |
| RM109730 | 1.84 |
| RM109011 | 1.84 |
| RM109521 | 1.83 |
| RM109796 | 1.82 |
| RM109135 | 1.81 |
| RM108951 | 1.81 |
| RM109490 | 1.75 |
| RM109048 | 1.73 |
| RM109023 | 1.72 |
| RM109005 | 1.70 |
| RM109638 | 1.70 |
| RM109480 | 1.69 |
| RM108954 | 1.68 |
| RM109142 | 1.68 |
| RM109398 | 1.66 |
| RM109271 | 1.65 |
| RM109894 | 1.63 |
| RM109264 | 1.62 |
| RM109016 | 1.62 |
| RM109589 | 1.62 |
| RM109644 | 1.60 |
| RM109811 | 1.60 |
| RM109093 | 1.58 |
| RM109012 | 1.53 |
| RM109797 | 1.52 |
| RM109311 | 1.52 |
| RM109025 | 1.51 |
| RM109823 | 1.51 |
| RM109591 | 1.50 |
| RM108974 | 1.50 |
| RM109672 | 1.48 |
| RM109062 | 1.48 |
| RM109630 | 1.44 |
| RM109201 | 1.44 |
| RM109050 | 1.44 |
| RM109147 | 1.43 |
| RM109170 | 1.42 |
| RM109119 | 1.42 |
| RM109001 | 1.41 |
| RM109391 | 1.40 |
| RM109406 | 1.36 |
| RM109464 | 1.34 |
| RM109695 | 1.31 |
| RM109738 | 1.30 |
| RM109321 | 1.29 |
| RM109633 | 1.29 |
| RM109341 | 1.28 |
| RM109734 | 1.15 |
| RM109153 | 1.15 |
| RM109181 | 1.12 |
| RM109810 | 1.12 |
| RM109652 | 1.11 |
| RM109733 | 1.08 |
| RM109603 | 1.01 |
| RM109613 | 0.98 |
| RM109570 | 0.98 |
| RM109336 | 0.96 |
| RM109022 | 0.92 |
| RM109735 | 0.88 |
| RM109561 | 0.84 |
| RM109479 | 0.72 |
| RM109731 | 0.64 |
| RM109182 | 0.63 |
| RM109053 | 0.61 |
| RM109583 | 0.54 |

### Example 9. C.203A>G Editing (Q68R) in SLC10A1 Transcripts in PHHs in Small Transfection Screens

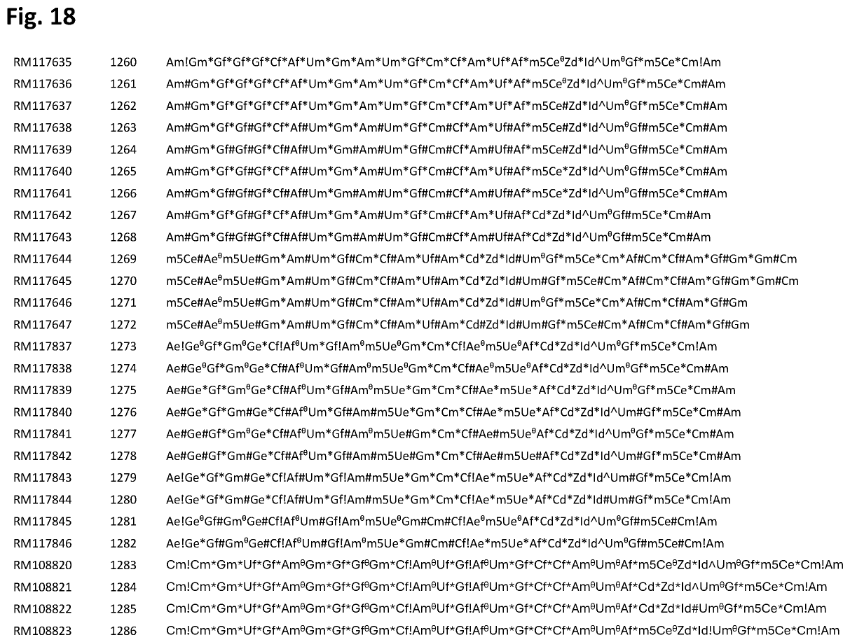

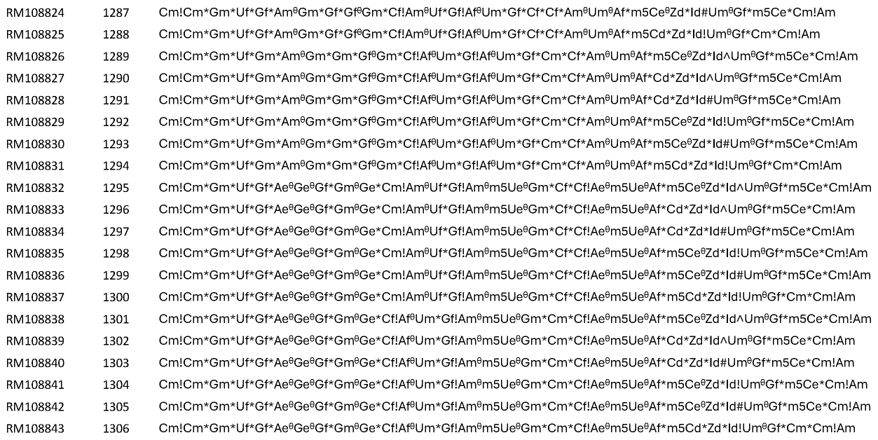

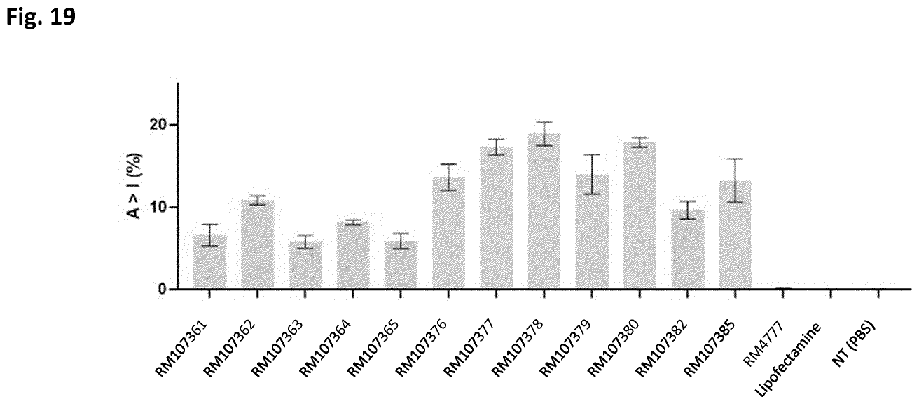

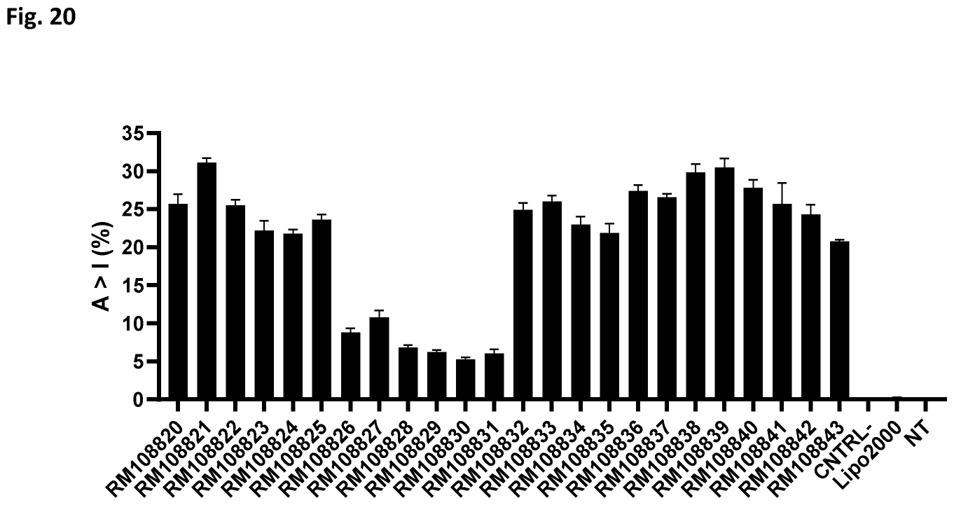

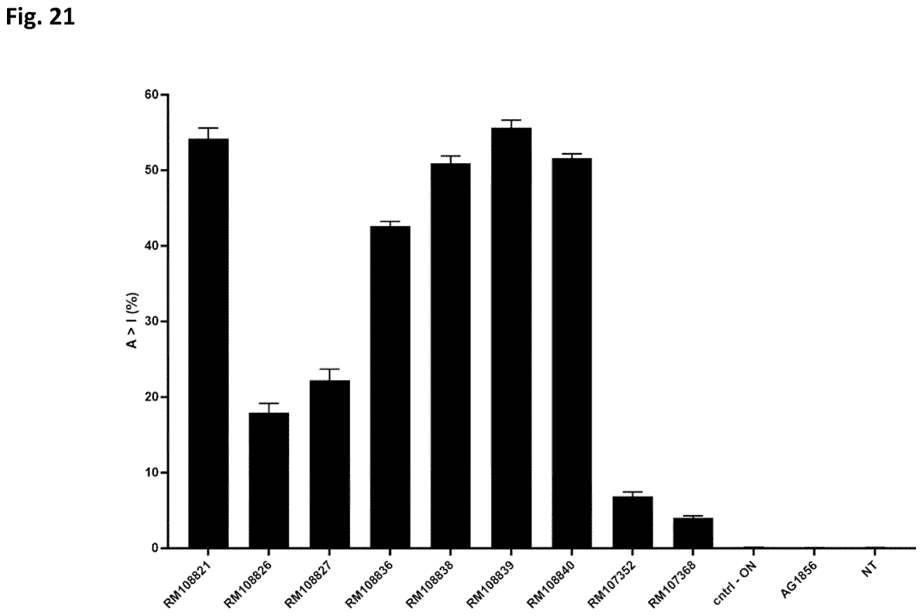

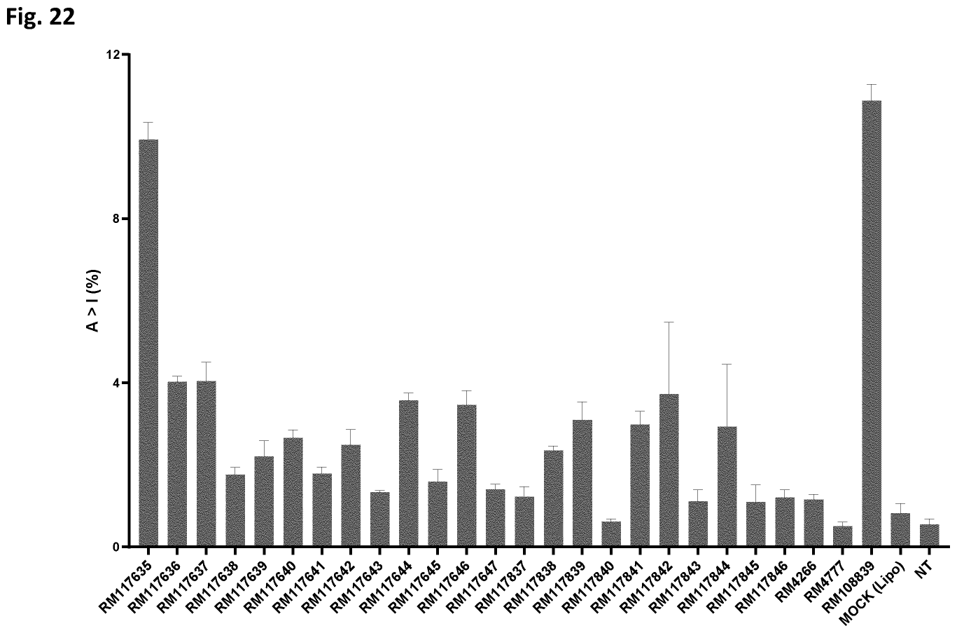

Several additional experiments as outlined in Example 8 were conducted with a variety of additional AONs, comprising a variety of chemistries, including alternative linkages, using Lipofectamine transfection or AG1856 co-treatment in PHHs. The details of the additionally tested AONs are provided in FIG. 18, showing the replacement of multiple MP and/or PNdmi linkages with PNms linkages (depicted with the hashtag #symbol), and a variety of position changes with 2′-F and 2′-MOE substitutions. Transfections, incubations, RNA isolation, cDNA generation and dPCR were performed as outlined above. FIG. 19 shows an experiment using RM107361, RM107362, RM107363, RM107364, RM107365, RM107376, RM107377, RM107378, RM107379, RM107380, RM107382, and RM107385 (with RM4777 as a negative control) and clearly indicate the preferred chemistries of for example RM107378 (which is comparable to RM107362 except for the presence of PNms linkages instead of PNdmi and MP linkages). FIG. 20 shows an experiment using RM108820 to RM108843 and suggests that the absence of 2′-MOE substituted nucleotides on the 5′ arm of the AON and the lower abundance of 2′-F substituted nucleotides (RM108826 to RM108831) negatively influences editing on the Q68R NTCP target. In contrast, using the 2′-MOE and 2′-F patterns in RM108838 to RM108843 appears to contribute to the editing efficiency, with RM108839 performing best (together with RM108821 that comprises significantly more 2′-F substituted nucleotides and no 2′-MOE substituted nucleotides on the 5′ arm of the AON). FIG. 21 shows an additional experiment using RM108821, RM108826, RM108827, RM108836, RM108838, RM108839, and RM108840 in comparison to the earlier tested (and relatively less performing) RM107352 and RM107368 AONs, applying AG1856 in a co-treatment (as outlined above), clearly indicating the reproducible high editing percentages obtained with RM108821 (SEQ ID NO: 1284) and RM108839 (SEQ ID NO: 1302) reaching levels above 55%. FIG. 22 shows a comparison with RM108839 with a variety of other AONs (RM117635 to RM117647 and RM117837 to RM117846) using transfection with lipofectamine, again showing that editing levels obtained with RM108839 were significantly high, and that RM117635 (SEQ ID NO: 1260) also performed well. Combining the teaching as disclosed herein, the AON of SEQ ID NO: 1302 is further modified to comprise

### Example 10. C.203A>G Editing (Q68R) in SLC10A1 Transcripts in Non-Human Primates Using AONs Encapsulated in Lipid Nanoparticles

In a next experiment it is investigated whether the best performing AONs from the in vitro screens can also yield editing of the c.203A position in vivo, in an endogenous wild-type SLC10A1 transcript, using endogenous ADAR enzymes, in the liver of non-human primates (NHP) providing a model for the therapeutically relevant Q68R change in humans. For this, RM107377 (SEQ ID NO:1173), RM107378 (SEQ ID NO:1174), and RM107385 (SEQ ID NO: 1181)—see FIG. 5, FIG. 15B, and FIG. 19—are encapsulated in a variety of lipid nanoparticles (LNP's) using standard methods known to the person skilled in the art. The EONs are administered at 4 time points with the following doses: 1 mg/kg, 2 mg/kg, 2 mg/kg, and finally 4 mg/kg. Liver biopsies are taken at different points in time after dosing to check for RNA editing using ddPCR generally following the protocols as outlined above, during a time course of >1 month. Several negative controls are taken along (a non-related AON and a non-treated subject). Bile acid concentration is assessed in the plasma of the treated NHP's as a functional read-out.

1. An antisense oligonucleotide (AON) that is capable of recruiting an endogenous ADAR enzyme in a human cell after the AON has formed a double-stranded complex with a region of a target RNA nucleic acid molecule in a cell, wherein the region comprises a target adenosine, wherein the nucleotide in the AON that is opposite the target adenosine is the orphan nucleotide, wherein the ADAR enzyme can deaminate the target adenosine into an inosine after binding to the double-stranded complex, and wherein the target RNA nucleic acid molecule is a transcript molecule of the human SLC10A1 gene encoding the Na⁺/Taurocholate Co-transporting Polypeptide (NTCP).

2. An AON according to claim 1, wherein the transcript molecule is a pre-mRNA or an mRNA molecule.

3. An AON according to claim 1 or 2, wherein the cell is a liver cell, preferably a hepatocyte.

4. An AON according to any one of claims 1 to 3, wherein the nucleotide numbering is such that the orphan nucleotide is number 0 and nucleotides are further positively (+) incremented towards the 5′ end and negatively (−) incremented towards the 3′ end, and wherein the orphan nucleotide is a deoxynucleotide comprising a cytosine, a cytosine analog, a uracil, or an iso-uracil.

5. An AON according to claim 4, wherein the orphan nucleotide is a deoxynucleotide comprising a cytosine analog, wherein the cytosine analog is a 6-amino-5-nitro-3-yl-2 (1H)-pyridone nucleobase.

6. An AON according to any one of claims 1 to 5, wherein the first nucleotide 3′ from the orphan nucleotide is a deoxyinosine when the nucleotide opposite this position is a cytidine in the target RNA nucleic acid molecule.

7. An AON according to any one of claims 1 to 6, wherein the AON is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length.

8. An AON according to any one of claims 1 to 7, wherein the AON comprises one or more modifications in the linkage moiety, which is each independently selected from the group consisting of: phosphorothioate (PS), phosphonoacetate, phosphorodithioate, methylphosphonate (MP), sulfonylphosphoramidate, (1,3-dimethylimidazolidin-2-ylidene) phosphoramidate (PNdmi), and mesyl phosphoramidate (PNms).

9. An AON according to claim 8, wherein the internucleoside linkage numbering in the AON is such that linkage number 0 is the linkage 5′ from the orphan nucleotide, and the linkage positions in the oligonucleotide are positively (+) incremented towards the 5′ end and negatively (−) incremented towards the 3′ end, and wherein linkage position-2 is an MP linkage or a PNms linkage.

10. An AON according to claim 8 or 9, wherein the linkage between the most terminal two nucleotides on the 5′ and/or 3′ terminus of the AON is a PNdmi linkage or a PNms linkage.

11. An AON according to any one of claims 1 to 10, wherein the AON comprises one or more nucleotides comprising a mono- or di-substitution at the 2′, 3′ and/or 5′ position of the ribose, each independently selected from the group consisting of: —OH; —F; substituted or unsubstituted, linear or branched lower (C₁-C₁₀)alkyl, alkenyl, alkynyl, alkaryl, allyl, or aralkyl, that may be interrupted by one or more heteroatoms; —O—, S—, or N-alkyl; —O—, S—, or N-alkenyl; —O—, S—, or N-alkynyl; -O—, S—, or N-allyl; -O-alkyl-O-alkyl; -methoxy; -aminopropoxy; -methoxyethoxy; -dimethylamino oxyethoxy; and -dimethylaminoethoxyethoxy.

12. An AON according to any one of claims 1 to 11, wherein the AON is covalently or non-covalently, directly or through a linker, bound to a GalNAc moiety.

13. An AON according to any one of claims 1 to 12, wherein the AON is covalently or non-covalently, directly or through a linker, bound to a triterpene glycoside, preferably AG1856.

14. An AON according to any one of claims 1 to 13, wherein the SLC10A1 gene is wildtype, and wherein the target adenosine is selected from the group consisting of:
the adenosine in the CAG codon coding for glutamine (Q) at position 68 of the NTCP protein, and wherein the deamination of the adenosine changes the amino acid to an arginine (R);
the first adenosine in the CAA codon coding for glutamine (Q) at position 261 of the NTCP protein, and wherein the deamination of the adenosine changes the amino acid to an arginine (R);
the adenosine in the GAG codon coding for glutamic acid (E) at position 257 of the NTCP protein, and wherein the deamination of the adenosine changes the amino acid to glycine (G); and
the first adenosine in the AAG codon coding for lysine (K) at position 314 of the NTCP protein, and wherein the deamination of the adenosine changes the amino acid to glutamic acid (E);
wherein the deamination of the target adenosine results in an NTCP protein that is impaired in its function to transport bile acids from portal circulation into the cell.

15. An AON according to claim 14, wherein the target adenosine is in the CAG codon coding for glutamine at position 68 of the NTCP protein, and wherein the AON comprises or consists of the sequence and modifications selected from the group consisting of SEQ ID NO: 150, 151, 152, 154, 156, 158, 159, 163, 164, 165, 166, 1127, 1128, 1129, 1130, 1131, 1133, 1137, 1138, 1139, 1140, 1141, 1142, 1143, 1144, 1145, 1146, 1150, 1151, 1152, 1153, 1154, 1155, 1156, 1157, 1158, 1159, 1160, 1161, 1167, 1168, 1169, 1170, 1171, 1172, 1173, 1174, 1175, 1176, 1177, 1178, 1180, 1181, 1182, 1183, 1260, 1283, 1284, 1285, 1286, 1287, 1288, 1295, 1296, 1297, 1298, 1299, 1300, 1301, 1302, 1303, 1304, 1305, and 1306.

16. An AON according to claim 14, wherein the target adenosine is in the GAG codon coding for glutamic acid at position 257 of the NTCP protein, and wherein the AON comprises or consists of the sequence and modifications selected from the group consisting of SEQ ID NO: 1193, 1194, 1197, 1198, 1199, 1200, 1201, 1202, 1203, 1204, 1205, 1206, 1207, 1208, 1209, 1210, 1211, 1212, 1213, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1253, and 1254.

17. A vector, preferably a viral vector, more preferably an adeno-associated virus (AAV) vector, comprising a nucleic acid molecule encoding an AON according to claim 1.

18. A nanoparticle delivery vehicle formulation comprising an AON according to any one of claims 1 to 16.

19. A nanoparticle delivery vehicle formulation according to claim 18, wherein the nanoparticle delivery vehicle is a lipid nanoparticle (LNP).

20. A pharmaceutical composition comprising an AON according to any one of claims 1 to 16, a vector according to claim 17, or a nanoparticle delivery vehicle formulation according to claim 18 or 19, and a pharmaceutically acceptable carrier.

21. An AON according to any one of claims 1 to 16, for use in the treatment of a disease caused by bile accumulation in the liver, such as cholestasis, primary sclerosing cholangitis (PSC), biliary atresia (BA), and liver cirrhosis.

22. Use of an AON according to any one of claims 1 to 16 in the manufacture of a medicament for the treatment of a disease caused by bile accumulation in the liver, such as cholestasis, PSC, BA, and liver cirrhosis.

23. A method of editing a human SLC10A1 pre-mRNA or mRNA molecule in a liver cell, preferably a hepatocyte, the method comprising contacting the SLC10A1 pre-mRNA or mRNA molecule with an AON capable of triggering an ADAR-mediated adenosine to inosine deamination, thereby editing the SLC10A1 pre-mRNA or mRNA molecule to encode an NTCP protein with a diminished, lowered, or loss of function in bile acid uptake, and wherein the AON is according to any one of claims 1 to 16.

24. A method of treating, ameliorating, or slowing down the progression of a disease caused by bile accumulation in the liver, such as cholestasis, PSC, BA, and liver cirrhosis, in a human subject in need thereof, the method comprising administering to said subject an AON according to any one of claims 1 to 16, a vector according to claim 17, or a nanoparticle delivery vehicle formulation according to claim 18 or 19, thereby contacting a SLC10A1 pre-mRNA or mRNA molecule in a cell of the subject, thereby effecting an ADAR-mediated adenosine to inosine deamination, thereby editing the SLC10A1 pre-mRNA or mRNA molecule to encode an NTCP protein with a diminished, lowered, or loss of function in bile acid uptake, thereby treating the subject.

25. An in vitro, ex vivo, or in vivo method for the deamination of a target adenosine in a human SLC10A1 pre-mRNA or mRNA molecule in a liver cell, preferably a hepatocyte, the method comprising the steps of:
(i) providing the cell with an AON according to any one of claims 1 to 16;
(ii) allowing uptake by the cell of the AON;
(iii) allowing annealing of the AON to the SLC10A1 pre-mRNA or mRNA molecule;
(iv) allowing an endogenous ADAR enzyme to deaminate the target adenosine in the SLC10A1 pre-mRNA or mRNA molecule to an inosine; and optionally
(v) using a functional read-out to identify the presence of the inosine in the SLC10A1 pre-mRNA or mRNA molecule.
